(19) United States
(12) Patent Application Publication
(10) Pub. No.: US 2026/0185038 A1
(43) Pub. Date: Jul. 2, 2026

(54) ENGINEERED SYNTROPHIC MICROBIAL CONSORTIA AND USES THEREOF

(71) Applicants: Eleftherios Papoutsakis, Newark, DE (US); Hyeongmin Seo, Newark, DE (US); Jonathan Otten, Newark, DE (US); Noah Willis, Newark, DE (US); John D. Hill, Newark, DE (US); Sofia Capece, Newark, DE (US)

(72) Inventors: Eleftherios Papoutsakis, Newark, DE (US); Hyeongmin Seo, Newark, DE (US); Jonathan Otten, Newark, DE (US); Noah Willis, Newark, DE (US); John D. Hill, Newark, DE (US); Sofia Capece, Newark, DE (US)

(73) Assignee: University of Delaware, Newark, DE (US)

(21) Appl. No.: 19/130,381

(22) PCT Filed: Nov. 15, 2023
(86) PCT No.: PCT/US2023/079827

§ 371 Date: May 15, 2025

Related U.S. Application Data
(60) Provisional application No. 63/425,554, filed on Nov. 15, 2022.
(60) Provisional application No. 63/506,235, filed on Jun. 5, 2023.

(51) Int. Cl. C12N 1/20; C12N 9/04; C12N 9/10; C12N 9/22; C12N 9/88; C12P 7/04; C12R 1/145

(52) U.S. Cl. CPC C12N 1/20; C12N 9/0006; C12N 9/1029; C12N 9/13; C12N 9/226; C12N 9/88; C12P 7/04; C12R 2001/145; C12Y 208/03009

(57) ABSTRACT

The present invention provides a recombinant microorganism having genetic modifications for enhancing production of metabolites. Also provided are a co-culture for producing metabolites, comprising the recombinant microorganism and an additional microorganism, and a method for producing metabolites by the co-culture.

### CROSS-REFERENCE TO RELATED APPLICATION

This application claims priority to U.S. Provisional Application No. 63/425,554, filed Nov. 15, 2022, and Provisional Application No. 63/506,235, filed Jun. 5, 2023, and the contents of each of which are incorporated herein by reference in their entireties for all purposes.

### REFERENCE TO U.S. GOVERNMENT SUPPORT

This invention was made with government support under contract awarded DE-AR0001505—UNIVERSITY OF DELAWARE by ARPA-E US Department of Energy. The United States has certain rights in the invention.

### FIELD OF THE INVENTION

The invention relates to an engineered syntrophic microbial consortium, for example, a co-culture of two microorganisms, including a recombinant microorganism, and uses thereof for producing metabolites and fixing CO₂.

### BACKGROUND OF THE INVENTION

Microbial production of metabolites though fermentation processes using carbohydrate substrates results in the formation and release of significant quantities of carbon dioxide (CO₂) due to the catabolism of the carbohydrates by microorganisms, resulting frequently in the loss of at least of one third of the carbon atoms of the carbohydrate as CO₂. As a result, the metabolite yields (that is the fraction of the carbohydrate mass conserved into the produced metabolites) are low, thus decreasing the process efficiency and commercial viability. To overcome this limitation, the goal of this invention is to develop a platform technology using synthetic syntrophic consortia of engineered Clostridium microorganisms to produce targeted metabolites as biofuels or chemicals with superior supraphysiological yields and efficiencies to achieve commercial viability. This can be accomplished using the disclosed engineered microorganisms and engineered consortium to enable fast and efficient utilization of renewable carbohydrates together with exogenous hydrogen (H₂) and CO₂ in a mixotrophic setting. Isopropanol is the first target metabolite. The synthetic consortium for simultaneous use of carbohydrates and exogenous H₂ and CO₂ is critical for achieving both the supraphysiological metabolite yields whereby all the carbon atoms of the carbohydrates are recovered into the produced metabolites, plus additional carbon atoms from the exogenous CO₂ are incorporated into the produced metabolites. These synthetic consortia uniquely enable modular design of fermentation processes to produce several commercially desirable metabolites via strain engineering and process

Thus, there remains a need for a syntrophic microbial consortium for producing desirable metabolites with enhanced CO₂ fixation to produce metabolites in a carbon neutral or carbon negative fermentation processes.

### SUMMARY OF THE INVENTION

The present invention relates to a co-culture of two microorganisms, including a recombinant microorganism, for producing metabolites, using exogenous carbohydrates, exogenous CO₂, and exogenous H₂.

According to a first aspect of the present invention, a method for producing metabolites by a co-culture is provided. The co-culture comprises a first microorganism and a second microorganism in a medium. The first microorganism is different from the second microorganism. The method comprises:
(a) growing the first microorganism and the second microorganism in the medium, wherein the medium comprises one or more exogenous carbohydrates, exogenous H₂, and exogenous CO₂;
(b) consuming the one or more exogenous carbohydrates by the first microorganism, and producing acetone, first acetate, CO₂ and H₂ by the first microorganism, wherein the first microorganism does not utilize for cell growth exogenous CO₂ or exogenous H₂, and does not produce butyrate, crotonate, butanol, butyraldehyde, beta-hydroxybutyrate or a long carbon chain length carboxylic acid;
(c) consuming the produced acetone, the produced CO₂, the produced H₂, the exogenous CO₂, and the exogenous H₂ by the second microorganism, and producing isopropanol, second acetate, and ethanol by the second microorganism, wherein the second microorganism does not produce CO₂ or H₂;
(d) consuming the second produced acetate by the first microorganism, and producing acetyl-CoA by the first microorganism; and
(e) forming cell membrane fusion by the first microorganism and the second microorganism, wherein electrons, the produced acetone, the produced CO₂ and the produced H₂ are transferred from the first microorganism into the second microorganism via the cell membrane fusion, and wherein the second produced acetate is transferred from the second microorganism into the first microorganism via the cell membrane fusion, whereby the co-culture produces the metabolites in the medium, wherein the metabolites comprise the produced acetone, the produced isopropanol, the produced ethanol, the produced first acetate, and the produced second acetate. The method may further comprise growing the co-culture anaerobically or micro-aerobically. The one or more exogenous carbohydrates may comprise starch, glucose, xylose, fructose, hemicellulose, arabinose, or a combination thereof.

According to the first aspect of the present invention, the first microorganism may be a recombinant Clostridium acetobutylicum (Cac), and the second microorganism may be Clostridium ljungdahlii (Clj). The one or more exogenous carbohydrates may consist of glucose. The method may further comprise consuming the glucose by the Cac and consuming the exogenous CO₂ by the Clj. At least 70% of the carbon atoms in the consumed glucose may be transferred into the produced isopropanol. Less than 25% of the carbon atoms in the consumed glucose may be transferred into the produced ethanol. The molar ratio of the consumed glucose to the consumed exogenous CO₂ may be from 2:1 to 1:6. Ten to eighty percent of the carbon atoms in the metabolites for each mol of the consumed glucose may be from the consumed exogenous CO₂.

According to the first aspect of the present invention, the Cac may overexpress one or more enzymes selected from the group consisting of Acetoacetyl-CoA: acetate/butyrate CoA transferases (CTFA&B), thiolase (THL), acetoacetate decarboxylase (ADC), secondary alcohol dehydrogenase (SADH), and a combination thereof. The Clj may overexpress one or more enzymes selected from the group consisting of Acetoacetyl-CoA: acetate/butyrate CoA transferases (CTFA&B), thiolase (THL), acetoacetate decarboxylase (ADC), secondary alcohol dehydrogenase (SADH), and a combination thereof.

According to the first aspect of the present invention, the Cac may overexpress one or more enzymes selected from the group consisting of THL of Escherichia coli (NCBI Reference Sequence: WP_000786547.1), CTFA&B of Escherichia coli (NCBI Reference Sequence: WP_000850540.1 and WP_000339065.1) or Clostridium acetobutylicum (NCBI Reference Sequence: WP_010890847.1 and WP_010890848.1), SADH of Clostridium beijerinckii (NCBI Reference Sequence: WP_077844196.1), and a combination thereof. The Clj overexpresses one or more enzymes selected from the group consisting of THL of Escherichia coli (NCBI Reference Sequence: WP_000786547.1), CTFA&B of Escherichia coli (NCBI Reference Sequence: WP_000850540.1 and WP_000339065.1) or Clostridium acetobutylicum (NCBI Reference Sequence: WP_010890847.1 and WP_010890848.1), SADH of Clostridium beijerinckii (NCBI Reference Sequence: WP_077844196.1), and a combination thereof.

According to the first aspect of the present invention, where spCas9 from Streptococcus Pyogenes (NCBI Reference Sequence: WP_010922251.1) is integrated into a chromosome of the Cac at the location of the endogenous lactate dehydrogenase gene (ldhA, Locus tag: CA_C0267), the method may further comprise disrupting the function of the ldhA in Cac. Endogenous 3-hydroxybutyryl-CoA dehydrogenase gene (hbd, Locus tag: CA_C2708) may be deleted from the chromosome or inactivated in the Cac. Endogenous short-chain-enoyl-CoA hydratase gene (crt, Locus tag: CA_C2712) may be deleted from the chromosome or inactivated in the Cac. Endogenous acyl-CoA dehydrogenase gene (bcd, Locus tag: CA_C2711) may be deleted from the chromosome or inactivated in the Cac. Where the medium comprises exogenous butyrate at a concentration of 1-200 mM, the method may further comprise enhancing the cell growth of the Cac. Where the medium further comprises exogenous crotonate at a concentration of 1-200 mM, the method may further comprise enhancing the cell growth of the Cac. Where the medium further comprises exogenous 3-hydroxybutyrate at a concentration of 1-200 mM, the method may further comprise enhancing the cell growth of the Cac. The method may further comprise passing the co-culture in a fresh medium without butyrate over at least 10 subcultures, and enhancing the cell growth of the Cac.

According to the first aspect of the present invention, the first microorganism may express a first fusion protein comprising a first half of a split fluorescence HaloTag protein and the second microorganism may express a second fusion protein comprising a second half of the split fluorescence HaloTag protein, a fluorescence signal may be generated upon contact of the first fusion protein with the second fusion proteins, and the method may further comprise detecting a fluorescence signal in the first microorganism or the second microorganism, wherein the presence of the fluorescence signal indicates a transfer of the first fusion protein from the first microorganism into the second microorganism or the second fusion protein from the second microorganism into the first microorganism. The method may further comprise determining the percentage of the first microorganism or the second microorganism showing the fluorescence signal.

According to the first aspect of the present invention, 50-100% of the carbon atoms in the metabolites may be from the one or more consumed exogenous carbohydrates.

According to the first aspect of the present invention, the co-culture may produce the metabolites with (i) a product yield higher than 0.5 Cmol of the produced isopropanol per Cmol of the one or more consumed exogenous carbohydrates, (ii) a product yield higher than 0.5 Cmol of the produced acetone per Cmol of the one or more consumed exogenous carbohydrates, and/or (iii) a product yield higher than 0.67 Cmol of the produced ethanol per Cmol of the one or more consumed exogenous carbohydrates.

According to a second aspect of the present invention, a method for producing metabolites by a co-culture is provided. The method comprises a first microorganism and a second microorganism in a medium, and the first microorganism is different from the second microorganism. The method comprises:
(a) growing the first microorganism and the second microorganism in the medium, wherein the medium comprises one or more exogenous carbohydrates, exogenous H₂, and exogenous CO₂;
(b) consuming the one or more exogenous carbohydrates by the first microorganism, and producing acetone, first acetate, butyrate, butanol, CO₂ and H₂ by the first microorganism, wherein the first microorganism does not utilize for cell growth the exogenous CO₂ or exogenous H₂;
(c) consuming the produced acetone, the produced CO₂, the produced H₂, the exogenous CO₂, and the exogenous H₂ by the second microorganism, and producing isopropanol, second acetate, and ethanol by the second microorganism, wherein the second microorganism does not produce CO₂ or H₂;
(d) consuming the second produced acetate by the first microorganism, and producing acetyl-CoA by the first microorganism; and
(e) forming cell membrane fusion by the first microorganism and the second microorganism, wherein electrons, the produced acetone, the produced CO₂ and the produced H₂ are transferred from the first microorganism into the second microorganism via the cell membrane fusion, and wherein the second produced acetate is transferred from the second microorganism into the first microorganism via the cell membrane fusion, whereby the co-culture produces the metabolites in the medium with (i) a product yield higher than 0.67 Cmol of a mixture of the produced isopropanol, the produced butyrate, the produced butanol, and the produced ethanol per Cmol of the one or more consumed exogenous carbohydrates, or (ii) a product yield higher than 0.67 Cmol of a mixture of the produced acetone, the produced butyrate, the produced butanol, and the produced ethanol per Cmol of the one or more consumed exogenous carbohydrates.

According to the third aspect of the present invention, a recombinant microorganism is provided. The recombinant microorganism is derived from a microorganism selected from the group consisting of Clostridium acetobutylicum, Clostridium ljungdahlii, Eubacterium limosum, Clostridium beijerinckii, Clostridium autoethanogenum, Acetobacterium woodii, Clostridium saccharolyticum, Clostridium butyricum and Clostridium carboxydivorans, and the recombinant microorganism overexpresses:
(a) one or more enzymes selected from the group consisting of Acetoacetyl-CoA: acetate/butyrate CoA transferases (CTFA&B), thiolase (THL), acetoacetate decarboxylase (ADC), secondary alcohol dehydrogenase (SADH), and a combination thereof; and/or
(b) one or more enzymes selected from the group consisting of THL of Escherichia coli (NCBI Reference Sequence: WP_000786547.1), CTFA&B of Escherichia coli (NCBI Reference Sequence: WP_000850540.1 and WP_000339065.1) or Clostridium acetobutylicum (NCBI Reference Sequence: WP_010890847.1 and WP_010890848.1), SADH of Clostridium beijerinckii (NCBI Reference Sequence: WP_077844196.1), and a combination thereof.

According to the third aspect of the present invention, the recombinant microorganism may be Clostridium acetobutylicum (Cac) or Clostridium ljungdahlii (Clj). spCas9 (NCBI Reference Sequence: WP_010922251.1) may be integrated into a chromosome of the recombinant microorganism at the location of endogenous lactate dehydrogenase gene (ldhA, Locus tag: CA_C0267). In one embodiment, endogenous 3-hydroxybutyryl-CoA dehydrogenase gene (hbd, Locus tag: CA_C2708) is deleted from the chromosome in the recombinant microorganism, and the recombinant microorganism does not produce butanol, butyrate, crotonate, butyraldehyde, beta-hydroxybutyrate or a long carbon chain length carboxylic acid. The recombinant microorganism may not utilize for cell growth exogenous CO₂ or exogenous H₂.

According to the fourth aspect of the present invention, a co-culture for producing metabolites is provided. The co-culture comprises the recombinant microorganism according to the third aspect of the present invention and an additional microorganism in a medium. The medium comprises one or more exogenous carbohydrates, exogenous H₂, and exogenous CO₂. The recombinant microorganism is different from the additional microorganism. The recombinant microorganism consumes the one or more exogenous carbohydrates, and produces acetone, first acetate, butanol, CO₂ and H₂. The additional microorganism consumes the produced acetone, the produced CO₂, the produced H₂, the exogenous CO₂, and the exogenous H₂, and produces isopropanol, second acetate, and ethanol. The recombinant microorganism consumes the second produced acetate and produces acetyl-CoA. The recombinant microorganism and the additional microorganism form cell membrane fusion, electrons, the produced acetone, the produced CO₂ and the produced H₂ are transferred from the recombinant microorganism into the additional microorganism via the cell membrane fusion, and the second produced acetate is transferred from the additional microorganism into the recombinant microorganism via the cell membrane fusion. The metabolites comprise the produced acetone, the produced isopropanol, the produced ethanol, the produced first acetate, the produced butanol, and the produced second acetate.

According to the fourth aspect of the present invention, the recombinant microorganism may be Clostridium acetobutylicum (Cac), and the additional microorganism may be Clostridium ljungdahlii (Clj). The one or more exogenous carbohydrates may consist of glucose. The Cac may consume the glucose. The Clj may consume the exogenous CO₂. At least 70% of the carbon atoms in the consumed glucose may be transferred into the produced isopropanol. Less than 25% of the carbon atoms in the consumed glucose may be transferred into the produced ethanol, the produced butanol, or a combination thereof. The molar ratio of the consumed glucose to the consumed exogenous CO₂ may be from 2:1 to 1:6. Ten to eighty percent of the carbon atoms in the metabolites for each mol of the consumed glucose may be from the consumed exogenous CO₂. The medium may further comprise exogenous butyrate, exogenous crotonate, and/or exogenous 3-hydroxybutyrate at a concentration of 1-200 mM.

According to the fourth aspect of the present invention, 50-100% of the carbon atoms in the metabolites may be from the one or more consumed exogenous carbohydrates.

According to the fourth aspect of the present invention, the co-culture may produce the metabolites with (i) a product yield higher than 0.5 Cmol of the produced isopropanol per Cmol of the one or more consumed exogenous carbohydrates, (ii) a product yield higher than 0.5 Cmol of the produced acetone per Cmol of the one or more consumed exogenous carbohydrates, and/or (iii) a product yield higher than 0.67 Cmol of the produced ethanol per Cmol of the one or more consumed exogenous carbohydrates.

According to a fifth aspect of the present invention, a co-culture for producing metabolites is provided. The co-culture comprises the recombinant microorganism according to the third aspect of the present invention and an additional microorganism in a medium. The medium comprises one or more exogenous carbohydrates, exogenous H₂, and exogenous CO₂. The recombinant microorganism is different from the additional microorganism. The recombinant microorganism consumes the one or more exogenous carbohydrates, and produces acetone, first acetate, butyrate, CO₂ and H₂. The additional microorganism consumes the produced acetone, the produced CO₂, the produced H₂, the exogenous CO₂, and the exogenous H₂, and produces isopropanol, second acetate, and ethanol. The recombinant microorganism consumes the second produced acetate and produces acetyl-CoA. The recombinant microorganism and the additional microorganism form cell membrane fusion, wherein electrons, the produced acetone, the produced CO₂ and the produced H₂ are transferred from the recombinant microorganism into the additional microorganism via the cell membrane fusion, and wherein the second produced acetate is transferred from the additional microorganism into the recombinant microorganism via the cell membrane fusion. The co-culture produces the metabolites with (i) a product yield higher than 0.67 Cmol of a mixture of the produced isopropanol, the produced butyrate, and the produced ethanol per Cmol of the one or more consumed exogenous carbohydrates, or (ii) a product yield higher than 0.67 Cmol of a mixture of the produced acetone, the produced butyrate, and the produced ethanol per Cmol of the one or more consumed exogenous carbohydrates.

### BRIEF DESCRIPTION OF THE DRAWINGS

FIG. 1 shows a syntrophic consortium of an engineered C. acetobutylicum (Cac) & WT C. ljungdahlii (Clj) to produce isopropanol (IPA) & ethanol from glucose according to one embodiment of the present invention.

FIGS. 2A-B show (A) design and construction, and (B) test of acetone production pathway in Cac M5. The metabolites were quantified after 75 h culture at 37° C. in 4 mL CGM medium containing 62 g/L glucose (344 mM) without pH adjustment. p95ace01a and p95ace02a have the same synthetic acetone production operons except the absence of adc gene from p95ace01a. The data represent three biological replicates.

FIGS. 3A-D show time dependent culture profiles of Cac M5 harboring either p95ace02a or p95ace02a_HHLH. The cells were cultured in CGM medium containing 60 g/L glucose without pH adjustment. (A) Cell growth, (B) glucose consumption, (C) butyrate and acetone production, and (D) acetate consumption. The data represent mean+/−standard deviation from three biological replicates.

FIGS. 4A-B show overexpressing a heterologous thiolase. (A) Metabolic pathway of acetone and ethanol production from the acetyl-CoA node. Ethanol and acetone production pathways compete at acetyl-CoA. (B) Amount of consumed glucose and final titers of ethanol and acetone from CACas9 Δhbd/p95ace02a (Cac_thl) and CACas9 Δhbd/p95ace02atoB (Eco_atoB) monoculture.

FIGS. 5A-F show comparison between CACas9 Δhbd/p95ace02a monoculture and coculture with Clj. (A) A profile of OD_{600 nm} (B) A profile of pH (C) A profile of glucose consumption (D) A profile of isopropanol production (E) A profile of acetone production (F) A profile of ethanol production. The data represent mean+/−standard deviation from two biological replicates.

FIGS. 6A-B show (A) A scheme of transwell coculture. Clj in the top (“TW”), Clj in the bottom with Cac in the top (“TWR”), and a mixed coculture control well (“CC”), and (B) A profile of glucose consumption by the coculture of CACas9 Δhbd/p95ace02a and Clj/p100ptaHalo in the transwell system.

FIGS. 7A-F show pH adjusted coculture of Clj/p100ptaHalo and CACas9 Δhbd/p95ace02a (Cac_thl) or CACas9 Δhbd/p95ace02atoB (Eco_atoB). (A) A profile of OD_{600 nm} (B) Coculture population profile of Cac_thl and Clj_Halo (C) Coculture population profile of Eco_atoB and Clj_Halo (D) A profile of serum bottle pressure (E) A profile of pH (F) Glucose consumption, ethanol production, and isopropanol production after 72 hours. The data represent mean+/−standard deviation from two biological replicates.

FIGS. 8A-B show metabolite titers from monoculture of CACas9 Δhbd harboring p95ace02atoB_sadH_cbei and coculture of Clj/p100ptaHalo with CACas9 Δhbd harboring p95ace02atoB_sadH_cbei. (A) Metabolite concentration after 48 hours of the cultures. (B) Product yields of the metabolites. The data represent mean values from at least three biological replicates.

FIGS. 9A-B show integration of spCas9 in the chromosome of Cac M5. (A) Structure of chromosomal integration location. Amplification of the location from a parental M5 strain results in 1.9 kb of PCR product, while 7.9 kb of PCR product is expected from a Cas9 integrated M5 strain. (B) An agarose gel picture of colony PCR products from seven transformant colonies and a parental M5 strain. The numbers above each well indicate individual transformant colonies. M, DNA ladder; C, parental M5 control. The red arrow indicates expected size (7.9 kb) of the integrated Cas9 cassette. The white arrow indicates expected size (1.9 kb) of the parental locus without the integrated Cas9 cassette.

FIGS. 10A-B show accelerated plasmid curing by lactose inducible antisense RNA against RepL. (A) Plasmid map of pGRNA_asRepL. (B) Plasmid curing of pGRNA plasmid with and without asRepL.

FIGS. 11A-B show genomic integration of a synthetic acetone production operon into the Cac chromosome. (A) A scheme of xylose inducible Cas9 genome editing for integration of a synthetic ctfA/B-adc operon into the Cac chromosome. (B) Colony PCR confirmation for successful integration of the acetone operon. The red box indicates expected product size (1.4 kb). The number from the gel picture indicates individual colonies. The M and C indicate DNA ladder and the negative control, respectively.

FIGS. 12A-B show integrating the xylose inducible cas9 cassette at lactate dehydrogenase (ldhA) location. (A) Genetic architecture of ldhA gene and chromosomally integrated xyIR-cas9 cassette. (B) Agarose gel picture of colony PCR screening. Seven colonies were PCR screened for successful integration of the xyIR-cas9 cassette (col1-7). A parental Cac 824 colony was screened as a control (824). Red arrow indicates band size corresponding to PCR product size of successful xyIR-cas9 cassette integration. Blue arrow indicates band size corresponding to PCR product size of parental strain locus without xyIR-cas9 cassette integration.

FIGS. 13A-D show comparison of cellular behaviors between CACas9 and Cac 824 (wild-type C. acetobutylicum). (A) Cell growth of Cac 824. (B) Cell growth of CACas9. (C) pH profile of the Cac 824 culture. (D) pH profile of the CACas9 culture.

FIGS. 14A-F show the effect of butyrate supplementation on the 4C metabolism disrupted Cac. (A) hbd gene deletion from the chromosome of CACas9. (B) CACas9 Δhbd cell growth with different amount of butyrate addition. (C) Amount of consumed glucose for 44 hours with different amount of butyrate addition. (D) Acetone production for 44 hours by CACas9 Δhbd with different amount of butyrate addition. (E) Butanol concentration after 44 hours by CACas9 Δhbd CACas9 Δhbd with different amount of butyrate addition. (F) A scheme of butyrate conversion into butanol.

FIG. 15 shows cell growth of CACas9 Δhbd in the absence of butyrate over the course of adaptive laboratory evolution.

FIGS. 16A-C show cell growth of (A) Escherichia coli, (B) C. ljungdahlii, and (C) C. saccharolyticum with or without 30 mM butyrate.

FIGS. 17A-H show (A) Biomass, and (B) pH profiles of CACas9 Δhbd p95ace02a_atoB monoculture and cocultures with various headspace compositions; (C) Headspace gas profiles for CACas9 Δhbd p95ace02a_atoB monoculture with N₂; (D) Headspace gas profiles for Cac-Clj coculture with N₂; (E) Headspace gas profiles for coculture with 20% N₂, 80% H₂, and (F) Headspace gas profiles for coculture with 20% CO₂/80% H₂; and (G) Soluble metabolite titers and (H) gas yields per glucose consumed.

FIG. 18 shows change in CO₂ incorporation and fixation rate for Clj monoculture with varied fructose feed rates and cell densities.

FIGS. 19A-B show coculture of CACas9 Δhbd/p95ace02a and Clj/p100ptaHalo with different fructose concentration. (A) Cell growth (B) Titers of glucose consumption, ethanol production, and isopropanol production after 72 hours.

FIG. 20 shows split-Halo assay for quantifying the physical interactions between Cac and Clj.

FIGS. 21A-B show profiles of metabolites from high cell density fermentation with sugar co-feeding. (A) Profile of liquid metabolites (B) H₂ and CO₂ profiles.

FIGS. 22A-D show repeated batch fermentation with high cell density. (A) Profile of biomass. (B) Profile of glucose consumption. (C) Profile of isopropanol and acetone production. (D) Profile of acetate and ethanol production.

FIGS. 23A-D show deletion of spo0A from CACas9Δhbd (n=2). (A) An agarose gel picture confirming spo0A deletion from the chromosome. The red arrow and box indicate the expected band size. (B) Growth profile of CACas9Δhbd and CACas9ΔhbdAspo0A (C) Final metabolite titers. (D) CO₂ evolution and electron flux.

FIGS. 24A-B show metabolite profiles of (A) monoculture of CACas9ΔhbdAspo0A containing p95ace02atoB and (B) coculture of CACas9ΔhbdAspo0A containing p95ace02atoB with Clj containing p100ptaHalo.

FIGS. 25A-D show the abrupt Cac cell growth cessation occurs at around 24 hours. (A, B) A profile of metabolites from the coculture of (A) CACas9Δhbd/p95ace02a (thl) or (B) CACas9Δhbd/p95ace02atoB (atoB) and Clj/p100ptaHalo. (C, D) Histograms of side-scatter (SSC) intensity in three replicates of both strains at 24 and 36 hours. The decrease of SSC indicates a universal decrease in cell viability. The data represent means+/−standard deviation from three biological replicates.

FIGS. 26A-B show CACas9Δhbd/p95ace02a (thl) and Clj/p100ptaHalo coculture exhibited direct cellular material exchange. (A) Flow cytometry histogram of the coculture CACas9Δhbd/p95ace02a and Clj/p100ptaHalo labeled by RNA-FISH (magenta, Cac) and HaloTag-OregonGreen (green, Clj) (B) Fluorescence microscopy of the coculture at 12 hours. The light blue arrows and circles indicate the double positive cells.

FIGS. 27A-B show (A) a scheme of redox sensitive Halo protein repression caused by direct interspecies electron transfer from Cac to Clj, and (B) flow cytometry fluorescent intensity comparison between Cac with redox sensitive fluorescent protein expression in coculture compared to monoculture.

FIGS. 28A-B show strategies to improve selectivity of acetone production by CACas9 Δhbd-based strains. (A) shows the reactions and enzymes involved in H₂ and IPA production. (B) shows the reactions for acetate uptake and acetone production.

FIG. 29 shows ethanol, acetone and H₂ production yields by CACas9 Δhbd strains overexpressing the electron flux related genes. The WT indicates the plasmid free control.

FIG. 30 schematically presents the metabolite production and substrate consumption for a Clj monoculture fed 5 g/L (33 mM) xylose with exogenous carbon dioxide and hydrogen in excess.

FIGS. 31A-B show a picture of the integrated, custom-designed glass bioreactor. (A) Four sidearms enable the connection of either a pH port or a cap that provides up to four possible inputs and outflows. The length of tubing protrusion into the vessel (if at all) or connection ports on each end are fully customizable. These vessels enable two-way pH control, sugar feeding, and cell resuspension and sampling in a chemical hood that is certified for gassing CO₂, CO, and H₂. (B) The gas control flowmeter panel with the gas distribution regulator, hub, alarm, and flashback arrestor in the background enables tight control and monitoring of gas dosing to each vessel.

FIGS. 32A-D show results from fermentations conducted in custom-designed glass bioreactors in which cocultures between CACas9 (-hbd) p95ace02atoB and Clj p100ptaHalo were grown and then spun down and resuspended in fresh growth medium every 24 hours. (A) OD trends as seen across four experimental resuspensions (vertical bars indicate resuspension occurrences). Each vessel used the same starting inoculum, but performance trends began to diverge over time. (B) Metabolites produced resuspensions from reactor 2. Ethanol is the main product, with ˜130 mM isopropanol attained after the fifth resuspension. Production of acetate and acetone is also observed. (C) A summarized plot of isopropanol and acetone production by each resuspension of vessel 2. Initially, acetone exceeded isopropanol in production, but as Clj began to increase in proportion throughout the coculture, isopropanol production increased as almost all acetone was converted. Additional Clj added at the first or second resuspension could have decreased acetone production sooner in the fermentation. (D) A summarized plot of glucose consumption and ethanol production by each resuspension in vessel 2. Ethanol remained the main product of this coculture, but we anticipate rebalancing the fermentation to favor isopropanol production.

FIGS. 33A-B show the characterization of the sadH overexpressing CACas9 Δhbd/p95IPA01atoB. (A) Metabolite profiles of CACas9 Δhbd/p95IPA01atoB under monoculture and coculture with Clj/p100ptaHalo. (B) Glucose consumption and carbon recovery for the 72 hours of coculture.

FIGS. 34A-D show the high cell density coculture of CACas9 Δhbd/p95ace02atoB and Clj/p100ptaHalo with repeated batch mode to simulate the cell retention system at the serum-bottle scale. (A) Time dependent profile of glucose, acetate, EtOH, IPA, and acetone. (B) Final titers of metabolites from individual batch rounds (RS). (C) IPA productivities from the individual batch rounds. (D) IPA and total 3C product (acetone +IPA) selectivity by the molar ratio over EtOH.

FIGS. 35A-B graphically present (A) how addition of 100 mM nitrate to the growth medium changes the metabolite production of a coculture between CACas9-hbd ace02atoB and CljGT Halo, and (B) the time profile of the headspace gas composition from these cocultures.

FIGS. 36A-C graphically present (A) the time profile of the carbon recovery of a coculture between CACas9-hbd ace02atoB and CljGT Halo, (B) a time profile of the soluble metabolite production of these cultures, and (C) a time profile of the headspace gas composition (vertical lines represent manual additions of exogenous gas).

FIGS. 37A-F show (A) flow cytometry results of a Cac M5 monoculture stained with RNAselect fluorescent dye, where the y-axis represents green fluorescence and the x-axis represents red fluorescence, (B) flow cytometry results a coculture between Cac M5 stained with RNAselect and Clj expressing the red fluorescent Halotag protein, (C) a monoculture of Clj expressing the red fluorescent Halotag protein, (D) a microscopy image of a coculture between Cac M5 stained with RNAselect and Clj expressing the red fluorescent Halotag protein showing only the green fluorescent channel, (E) the same microscopy image of a coculture between Cac M5 stained with RNAselect and Clj expressing the red fluorescent Halotag protein showing overlaying the green and red fluorescent channels, and (F) the same microscopy image of a coculture between Cac M5 stained with RNAselect and Clj expressing the red fluorescent Halotag protein showing only the red fluorescent channel.

FIGS. 38A-B show metabolite profiles of monoculture and coculture of M5Cas9 Δhbd/p95ace02a (M5Cas9 Δhbd_p95ace02a) and Clj/p100ptaHalo (Clj_Halo). (A) Metabolite titers (concentrations, mM) after 24 hours without pH control. (B) Time-dependent profiles of cell growth (OD) and the two major metabolite products (lactate and acetate) for 72 hours with pH control. (C) Final titers of ethanol, acetone, and IPA after 72 hours with pH control.

FIGS. 39A-D show (A) the setup of transwell cocultures used for RNAseq experiments, (B) the number of genes that were up or down regulated with statistical significance for Cac and Clj at the 2, 4, and 11 hr timepoints, (C) the number of specific genes differentially expressed at one or more timepoints for Cac, and (D) the number of specific genes differentially expressed at one or more timepoints for Clj.

### DETAILED DESCRIPTION OF THE INVENTION

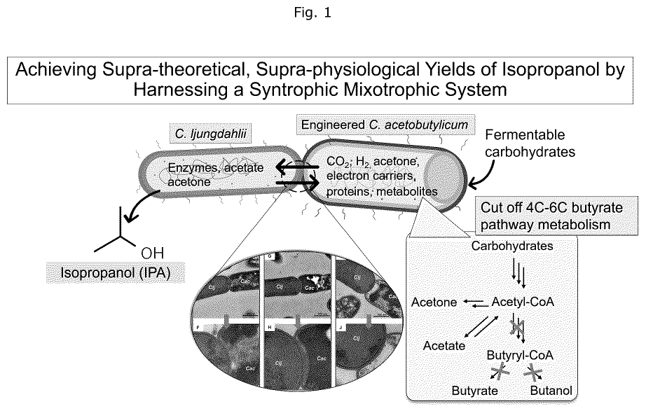

The present invention relates to a recombinant microorganism, an engineered syntrophic microbial consortium, for example, a co-culture of two or more microorganisms, including the recombinant microorganism, and uses of the recombinant microorganism and the engineered syntrophic microbial consortium for producing metabolites and fixing CO₂. The present invention is based on the inventors' discovery of a two-strain syntrophic consortium (FIG. 1): C. acetobutylicum (Cac) and C. ljungdahlii (Clj). Cac efficiently utilizes a broad spectrum of carbohydrates including all simple five and six-carbon (C) sugars, oligosaccharides, and xylans, and normally produces butanol, acetone, ethanol, butyrate, acetate, H₂ and CO₂. Cac cannot use CO₂ and H₂ as the sole carbon and energy sources for cell growth. Clj is an acetogen using the Wood-Ljungdahl pathway, needs CO₂ and H₂ for growth and survival as it does not use major biomass mono/oligosaccharides, for example, glucose, and produces largely acetate and some ethanol. Clj uses the CO₂ and H₂ produced by Cac to grow, plus additional externally supplied CO₂ and H₂, but, significantly, it can also directly extract electrons, for example, in the form of electron carriers, and metabolites from Cac, to enhance its own growth and survival. Cac also benefits from Clj through the direct heterologous cell to cell interactions as we disclose in this invention application. The mutual synergies of this system lead to enhanced and stable metabolic behavior. Clj converts up to 100% of the acetone produced by Cac into isopropanol (IPA). Using an engineered Cac strain producing only acetone with some ethanol (FIG. 1), the inventors have developed a coculture of an engineered Cac strain and Clj to use glucose and other fermentable carbohydrates as substrates to produce products, including IPA as the main product and ethanol as a co-product in amounts of greater than 80% and less than 20%, respectively, based on the total weight of the products. This two-strain syntrophic consortium address technological challenges, including efficient and complete utilization of all carbon and electrons in a sugar substrate; additional 0-6 mol CO₂ utilized per mol glucose, with H₂ as the electron donor; operational stability; and flexibility of substrate source and of products, so that many biomass sugars can be used and several products produced.

The terms “microorganism” and “microbe” are used herein interchangeably and refers to a bacterium.

The terms “recombinant microorganism” and “engineered microorganism” are used herein interchangeably and refer to a non-naturally occurring microorganism.

The term “culture” as used herein refers to a preparation of a microorganism grown in a medium, which is also referred to as a culture medium. Where two or more different types of microorganisms are grown in the same medium, the culture may also be referred to as “coculture” or “co-culture.” Where only one type of microorganisms is grown in the same medium, the culture may also be referred to as “monoculture.” Where at least one microorganism in the culture is recombinant, the culture is an engineered culture.

The term “substrate” as used herein refers to a substance, for example, a carbohydrate or gas (e.g., CO₂ or H₂), that a microorganism consumes to produce a metabolite. Where a substrate can be broken down by the microorganism into one or more other substances, the substrate is referred to as being fermentable. When a microorganism converts a substrate biochemically regardless of cellular energy change or cell growth, the substrate is deemed consumed by the microorganism. When a microorganism converts a substrate biochemically such that there is generation of cellular energy required for cell growth, the substrate is deemed as utilized by the microorganism for cell growth. In one embodiment, the microorganism utilizes a substrate (e.g., CO₂ or H₂) for cell growth. In another embodiment, the microorganism does not utilize a substrate (e.g., CO₂ or H₂) for cell growth.

The term “metabolite” as used herein refers to a substance produced by a microorganism from a substrate. Examples of the metabolites include isopropanol, acetone, ethanol, acetate, acetoacetate, butyrate, crotonate, beta hydroxybutyrate, butanol, acetoin, 2,3 butanediol, long carbon chain length carboxylic acids and their corresponding alcohols, activated intracellular metabolites such as acetyl-CoA, acetoacetyl-CoA, butyryl-CoA, intermediates of the glycolytic pathway, pyruvate, intermediates of the tricarboxylic acid (TCA) cycle, which is also known as Krebs cycle and citric acid cycle, amino acids, and nucleic acid intermediates.

The term “mixotroph” as used herein refers to a microorganism capable of using different sources of energy and carbon.

The term “syntrophic microbial consortium” as used herein refers to a stable co-existence of two or more microbes, in which at least one microbe feeds off at least one product of at least one other microbe. The metabolic activities of the consortium benefit all of the microbes to enable stable co-existence in the presence of one or more substrates. The microbes in a syntrophic microbial consortium that simultaneous consume a mixture of substrates, for example, sugars (e.g., carbohydrates) and gases (e.g., CO₂, H₂, and/or CO) have both mixotrophic and syntrophic characteristics.

The term “fermentation system” as used herein refers to a culture in which a microorganism breaks down an exogenous fermentable substrate and produces one or more metabolites. Such a metabolite is also referred to as being produced endogenously.

The term “RNA Sequencing” (also called RNA Seq) refers herein to the high coverage, high throughput next-generation sequencing method to identify the presence and number of RNA transcripts throughout the entire transcriptome of a microorganism. A transcriptome includes the entirety of coding and non-coding RNA transcripts (total RNA) of a microorganism. RNA Seq can be used to measure the expression of thousands of genes and to compare the expression levels across multiple conditions to determine differential gene expression. To begin the RNA Seq process, total RNA is harvested from the cells and the RNA molecules of interest (typically mRNA to determine coding RNA) are purified. Single stranded RNA can be converted to double stranded complementary DNA, followed by library preparation, and analysis by the next generation sequencing method. The data output of the sequencing method are reads, which can be mapped to the microorganism's genome. The number of reads that align to a particular region on the genome represent the transcriptional activity of the gene.

The term “Transcriptional changes” refers herein to the enhancement or repression of the transcription of DNA to mRNA and all RNA within the cell. These changes are referred to as transcriptional changes, Expression of mRNA of certain genes or non mRNA molecules (such as small noncoding RNAs and ribosomal RNA) within the cells is dependent on environmental factors and timing in development of the cell. The microorganism's DNA contains a variety of DNA binding motifs that recruit activating proteins or repressor proteins to control expression of genes and their number of mRNA transcripts.

The term “gene knock-out” as used herein refers to the process by which a gene-coding segment of DNA within the microorganism's genomic DNA is specifically targeted and deleted entirely, while the rest of the genetic DNA retains its integrity. As a result, the expression of mRNA from the targeted gene will be permanently prevented in the microorganism. Commonly used methods for gene-knockouts include CRISPR/Cas9 in which a small guide RNA (sgRNA) is designed specifically for the gene of interest for deletion, and upon induction of Cas9, the sgRNA directs the Cas9 endonuclease subunit to generate a double-stranded break in the microorganism's genomic DNA. The repair of the genomic DNA is facilitated by homology-directed repair with homologous regions of the microorganism's genomic DNA that immediately flank the targeted gene. The homologous regions can be provided on a plasmid DNA template and help to facilitate a double-crossover event in the genomic DNA to fully excise the targeted gene. There are also several molecular tools for gene knock outs, such as the lambda red system, use of heterologous counterselection markers for identification of allelic exchange events, and targeted gene inactivation with replicative plasmids capable of double-crossover chromosomal integration. Herein, the gene knockouts from a strain are denoted as either “Agene name” or “(-)gene name.”

Lambda red technology is well-established for E. coli, allowing for the introduction of linear DNA as a template for DNA repair, rather than plasmid-based homologous recombination methods. In E. coli, three lambda red bacteriophage proteins help to stabilize and inhibit digestion of the linear DNA template introduced to the cell. To date, the RecT protein from Clostridium perfringens has been found to be similar to lambda red proteins, but its recombineering capabilities have since just been demonstrated in ssDNA. Further optimization of this recombineering strategy could aid the efficiency of generating gene knock-outs or knock-ins in Clostridium species (Charubin, Bennett et al. 2018), drastically reducing the molecular cloning steps that occur.

The system employed by Al-Hinai, et al (Al-Hinai, Fast et al. 2012) to facilitate gene replacement via allelic exchange utilizes a codon-optimized mazF toxin gene from E. coli4. When expressed, mazF cleaves mRNA at ACA sequences, inhibiting growth and promoting cell death. However, expression of mazF under a lactose-inducible promoter (P bgaL), was shown to be tightly regulated, with cells harboring mazF growing abundantly on plates without lactose and showing no growth on plates with lactose supplementation. As a result, mazF under the control of P bgaL was cloned onto a plasmid with 1 kb homology arms for the gene of interest, surrounding a Thiamphenicol resistance marker flanked by FRT sites (FLP-FRT recombination is possible to later excise the antibiotic resistance marker). Double-crossover events, in which the genomic gene of interest has been replaced by the thiamphenicol resistance marker, can be selected for in cells able to grow on lactose-supplemented plates. One advantage of this system is the use of mazF as a counter-selection marker does not require prior mutations or auxotrophy in the strain one is working with.

The method for targeted gene inactivation with replicative plasmids capable of double-crossover chromosomal integration was first successfully carried out in solventogenic clostridia for the chromosomal spo0A gene by L. M Harris, et. al. (Harris, Welker et al. 2002). A plasmid was constructed with both thiamphenicol and erythromycin resistance markers, along with homologous regions of spo0A to employ crossover events to inactivate the spo0A gene. First, a single crossover integration of the entire methylated plasmid was carried out at the site of one homologous region of spo0A recognized on the plasmid. Next, a second crossover event between two 10 nucleotide homologous sequences in the plasmid excised the plasmid backbone including the region conferring thiamphenicol resistance. As a result, the final spo0A-inactivated strain with partial (2.1 kb) insertion of the plasmid conferred only erythromycin resistance to the strain. The final strain could be selected based on its ability to grow solely on erythromycin supplemented plates, but not those supplemented with thiamphenicol.

The term “gene replacement” as used herein refers to substitution of a part or all of the DNA sequence of a targeted gene in the genome of a microorganism with the DNA sequence of a desired gene. Gene replacement can be accomplished by cloning the sequence to be inserted into the genome between two homologous regions of DNA that flank the targeted gene to be excised/knocked-out. Upon excision of the targeted gene for removal, the cell's repair machinery will utilize the DNA template containing homologous regions surrounding the new genetic sequence to be inserted, performing a double cross-over event to repair the DNA.

The term “gene disruption” as used herein refers to a change to a gene such that the gene is inactivated or no longer functional in a microorganism through, for example, insertion of novel DNA sequences, partial deletion of the gene by CRISPR/Cas9, homologous recombination or other strategies. However, contrary to gene knock-out, some elements of the original gene may still exist within the genome of the microorganism but can no longer be transcribed to produce the mRNA or protein of the disrupted gene. Gene disruption applies to pseudo-genes, genes coding for ribosomal RNAs, tRNAs and other non-coding regulatory RNAs. Gene disruption can be generalized to disruption of non-coding regions of a cell's genomic DNA. Pseudogenes are nonfunctional segments of DNA that resemble functional genes or genes for which some elements characterizing proper genes (e.g., a promoter or a ribosome binding site or a transcriptional terminator) are missing.

The term “deleted” when applied to a gene coding DNA sequence refers herein to removal of a portion or all of the DNA sequence of a gene such that the function of that gene is lost. For example, the gene upon “deletion” is unable to lead to the synthesis of a functional gene product including a functional mRNA and the protein it codes for, or loss of function of a non-coding RNA such as a ribosomal RNA, transfer RNA (tRNA) or small or large non-coding RNA. A gene can be deleted by “gene knock-out”, “gene replacement”, “gene inactivation”, “gene disruption” or alteration of the polynucleotide sequence of the structural or regulatory elements of the gene. A regulatory sequence (such as a promoter or a transcriptional terminator) is a segment of a nucleic acid molecule which is capable of increasing or decreasing the expression of specific genes within a microorganism. Expression refers to the production of one or more RNA molecules coded by the DNA sequence of a gene. Structural elements of the gene here are defined as those other than the regulatory sequences of a gene.

The term “catabolite repression” as used herein refers to a regulatory mechanism by which the expression of genes required for a microorganism to utilize a secondary source of carbon and/or energy, which may be a substrate less preferred by the microorganism, is prevented by the presence of a preferred substrate, a primary source of carbon and/or energy.

The term “carbon negative” as used herein refers to carbon metabolism of a fermentation system using a fermentable carbohydrate to produce non-CO₂ metabolic products, and the number of whose carbon atoms is larger than the number of carbon atoms in the fermentable carbohydrate as being used, not supplied. This may be achieved by the ability of the fermentation system to utilize exogenous CO₂ or other non-carbohydrate chemicals and to utilize the endogenously produced CO₂ due to carbohydrate utilization.

The term “carbon neutral” as used herein refers to carbon metabolism of a fermentation system using a fermentable carbohydrate to produce non-CO₂ metabolic products, and the number of whose carbon atoms is equal to the number of carbon atoms in the fermentable carbohydrate as being used, not supplied. This may be achieved by the ability of the fermentation system to utilize exogenous CO₂ or other non-carbohydrate chemicals and to utilize the endogenously produced CO₂ due to carbohydrate utilization.

The term “anaerobic culture” as used herein refers to a culture of one or more microbial species that are poisoned by oxygen. Oxygen levels must be managed by sparging of other gases, the capture and elimination of oxygen, or some other method. The term “anaerobe” refers to one or more of these microorganisms that cannot tolerate oxygen. Where a microorganism is growth in the absence of oxygen, the microorganism is grown anaerobically.

The term “aerobic culture” as used herein refers to a culture of one or more microbial species that require oxygen because they lack the capability for anaerobic respiration. The term “aerobe” refers to one or more of these microorganisms that require oxygen.

The term “microaerobic culture” as used herein refers to a culture of a microorganism that can grow on various carbon and energy sources in a medium containing oxygen at a low concentration, for example, in the range of about 1-10 v/v %. The microorganism may or may not be able to use oxygen, but it can tolerate oxygen to the extent that it grows in the presence of the oxygen. Such a microorganism is aerotolerant.

The term “microaerobe” as used herein refers to a microorganism that can grow under “microaerobic culture” conditions. Where a microorganism is grown in a medium containing oxygen at a low concentration of, for example, about 1-10 v/v %, the microorganism is deemed grown anaerobically.

The term “acetogen” as used herein refers to a microorganism that produces acetate as an end product of anaerobic respiration through the Wood-Ljungdahl pathway.

The term “Wood-Ljungdahl pathway” as used herein refers the microbial pathway used by anaerobic microorganisms known as autotrophs because they grow using CO₂ and H₂ as the sole carbon and energy sources. The microorganisms use hydrogen as an electron donor, and carbon dioxide as an electron acceptor and as a building block for biosynthesis. The pathway is also known as reductive acetyl-CoA and is the main mechanism for energy conservation and for synthesis of acetyl-CoA and cell carbon from CO₂ (Ragsdale and Pierce 2008).

Clostridium kluyveri, as referenced herein, is an anaerobic, Gram-positive, spore-forming microorganism that can grow anaerobically on ethanol and/or some other alcohols and acetate and/or some other carboxylic acids and produces butyrate, and long carbon-chain carboxylic acids, C5-C8 carboxylic acids.

The term “long carbon chain length carboxylic acid” as used herein to refer to a linear or non-linear carboxylic acid comprised of more than four carbon atoms. Examples of the long carbon chain length carboxylic acids include hexanoate (caproate), pentanoate, heptanoate and octanoate. In microbial metabolism the production of such long carbon-chain length carboxylic acids is referred to as chain elongation and is typically carried out by microorganisms such as Clostridium kluyveri. Such change elongation can be engineered into other microbes using genetic engineering.

The term “butyrate” as used herein refers to butanoic acid, also known as butyric acid, or any salt thereof, for example, sodium butyrate, potassium butyrate, or magnesium butyrate.

The term “crotonate” as used herein refers to crotonic acid, also known as (2E)-but-2-enoic acid, or any salt thereof, for example, sodium crotonate.

The term “acetate” as used herein refers to acetic acid, also known as ethanoic acid, and any salt thereof, for example, sodium acetate, ammonium acetate, or potassium acetate.

The term “beta hydroxybutyrate” as used herein refers to “beta hydroxybutyric acid”, or any salt thereof.

The term “direct electron exchange” as used herein refers to transfer of electrons in the form of electron carrier molecules or electron rich molecules from one microorganism to another microorganism through direct physical contact and without releasing the electron carrier or electron rich molecules into the culture medium where the two microorganisms are growth. Examples of the electron carrier molecules include NADH, NADPH, FADH. Examples of electron carrier proteins include ferredoxin, thioredoxin, rubredoxin, and cytochromes. Examples of species-specific electron carrier proteins include EtfA and EtfB proteins of, for example, Clostridium acetobutylicum. Examples of electron rich molecules include hydrogen (H₂), formate (formic acid), and methanol. Direct physical contact may include close contact of cell walls and cell membranes enabling electron transport through electron carrier proteins in the cell membrane or cytoplasmic exchange of cellular material such as proteins, RNAs and DNA as mediated by heterologous cell fusion or cell-wall fusion, cell-membrane fusion, or a combination thereof. The term “direct electron exchange” excludes electron transfer through nanowires.

The term “direct metabolite transfer” as used herein refers to transfer of metabolites from one microorganism to another microorganism through direct physical contact and without releasing the metabolites into the culture medium where the two microorganisms are grown. Direct physical contact may include close contact of cell walls and cell membranes enabling direct transport or transfer of the metabolites or through cytoplasmic exchange of cellular material, for example, the metabolites, as mediated by heterologous cell fusion, cell-wall fusion, cell-membrane fusion, or a combination thereof.

The terms “cell fusion” and “membrane fusion” between two different microorganisms or between cells of the same microorganism are used herein interchangeably and refer to fusion of their cell wall (if they have one) and cell membrane that enables direct transfer of a cellular material including electrons, for example, in the form of electron carrier molecules, metabolites, proteins, RNAs, and a plasmid DNA. In a coculture setting, the fusion events are dynamic, and the actual fusion events change with time from very few to many. As fusion events are dynamic and may not last long enough to be able to capture them by the currently available assays like flow cytometry, electron and fluorescent microscopies. The terms “cell fusion,” “membrane fusion,” and “cell-wall fusion,” whether heterologous (among two different microorganisms) or homologous (among cells of the same microorganism), are used herein to mean that at least a fraction of the cells, for example, from about 1% to about 100% are experiencing or have experienced these “fusion” events during a coculture or monoculture.

The term “hybrid cell” as used herein refers to a cell formed transiently or permanently cell by two different microorganisms. The hybrid cell exhibits some properties or components (e.g., proteins, nucleic acids or other cellular material) from both microorganisms, cannot be identified uniquely as being one or the other microorganism, and is capable of replicating for generations. Hybrid cells may be a small or a large fraction of the cells in a co-culture and co-exist with one or both of the parent microorganisms from where they arise.

### First Aspect of the Invention

According to the first aspect of the present invention, a method for producing metabolites by a co-culture is provided. The co-culture comprises a first microorganism and a second microorganism in a medium. The first microorganism is different from the second microorganism. The first microorganism does not utilize for cell growth exogenous CO₂ or exogenous H₂, and does not produce butyrate, crotonate, butanol, butyraldehyde, beta-hydroxybutyrate or a long carbon chain length carboxylic acid. The second microorganism does not produce CO₂ or H₂. The medium comprises one or more exogenous carbohydrates, exogenous H₂, and exogenous CO₂. The method comprises growing the first microorganism and the second microorganism in the medium; consuming the one or more exogenous carbohydrates by the first microorganism, and producing acetone, first acetate, CO₂ and H₂ by the first microorganism; consuming the produced acetone, the produced CO₂, the produced H₂, the exogenous CO₂, and the exogenous H₂ by the second microorganism, and producing isopropanol, second acetate, and ethanol by the second microorganism; consuming the second produced acetate by the first microorganism, and producing acetyl-CoA by the first microorganism; and forming cell membrane fusion by the first microorganism and the second microorganism. Electrons, the produced acetone, the produced CO₂ and the produced H₂ are transferred from the first microorganism into the second microorganism via the cell membrane fusion, and the second produced acetate is transferred from the second microorganism into the first microorganism via the cell membrane fusion. As a result, the co-culture produces the metabolites in the medium. The metabolites comprise the produced acetone, the produced isopropanol, the produced ethanol, the produced first acetate, and the produced second acetate.

The method may further comprise growing the co-culture anaerobically. The medium may comprise oxygen.

The method may further comprise growing the co-culture micro-aerobically. The medium may comprise oxygen at a concentration of about 1-20, 1-15, 1-10, 1-5, 1-4, 1-3, 1-2, 2-20, 2-15, 2-10, 2-5, 2-4, 2-3, 3-20, 3-15, 3-10, 3-5, 3-4, 4-20, 4-15, 4-10, 4-5, 5-20, 5-15, 5-10, 10-20, 10-15 or 15-20 v/v %.

The one or more exogenous carbohydrates may comprise starch, glucose, xylose, fructose, hemicellulose, arabinose, or a combination thereof. The one or more exogenous carbohydrates may consist of glucose.

The first microorganism may be derived from a microorganism selected from the group consisting of Clostridium acetobutylicum, Clostridium beijerinckii, Clostridium saccharolyticum, and Clostridium butyricum that have similar biological traits, for example, the ability to use a broad spectrum of carbohydrates as carbon and energy sources, to produce the same or similar set of metabolites, and their inability to use CO₂ and H₂ as the sole sources for growth.

The second microorganism may be derived from a microorganism selected from the group consisting of Clostridium ljungdahlii, Clostridium autoethanogenum, Acetobacterium woodii, Eubacterium limosum, and Clostridium carboxydivorans that have the similar biological traits, as they all use the Wood-Ljungdahl pathway, are mesophiles, and have similar metabolic pathways for biomass synthesis.

The first microorganism may be a recombinant Clostridium acetobutylicum (Cac), and the second microorganism may be Clostridium ljungdahlii (Clj). The one or more exogenous carbohydrates consist of glucose. The method may further comprise consuming the glucose by the Cac, and consuming the exogenous CO₂ by the Clj. At least about 50%, 60%, 70%, 80% or 90%, or about 50-99%, 50-90%, 50-80%, 50-70%, 50-60%, 60-99%, 60-90%, 60-80%, 60-70%, 70-99%, 70-90%, 70-80%, 80-99%, or 80-90% of the carbon atoms in the consumed glucose may be transferred into the produced isopropanol. Less than about 40%, 30%, 25%, 20%, 10% or 5%, or about 0.1-40%, 0.1-30%, 0.1-25%, 0.1-20%, 0.1-10%, 0.1-5%, 0.1-1%, 0.1-0.5%, 0.2-40%, 0.2-30%, 0.2-25%, 0.2-20%, 0.2-10%, 0.2-5%, 0.2-1%, 0.2-0.5%, 0.5-40%, 0.5-30%, 0.5-25%, 0.5-20%, 0.5-10%, 0.5-5%, 0.5-1%, 1-40%, 1-30%, 1-25%, 1-20%, 1-10%, 1-5%, 5-40%, 5-30%, 5-25%, 5-20% or 5-10% of the carbon atoms in the consumed glucose may be transferred into the produced ethanol. The molar ratio of the consumed glucose to the consumed exogenous CO₂ may be from about 10:1 to about 1:10, from about 2:1 to about 1:6, or from about 2:1 to about 1:2. About 1-95%, 1-90%, 1-80%, 1-70%, 5-95%, 5-90%, 5-80%, 5-70%, 10-95%, 10-90%, 10-80%, 10-70%, 20-95%, 20-90%, 20-80% or 20-70% of the carbon atoms in the metabolites for each mol of the consumed glucose may be from the consumed exogenous CO₂.

The Cac may overexpress one or more enzymes selected from the group consisting of Acetoacetyl-CoA: acetate/butyrate CoA transferases (CTFA&B), thiolase (THL), acetoacetate decarboxylase (ADC), secondary alcohol dehydrogenase (SADH), and a combination thereof.

The Clj may overexpress one or more enzymes selected from the group consisting of Acetoacetyl-CoA: acetate/butyrate CoA transferases (CTFA&B), thiolase (THL), acetoacetate decarboxylase (ADC), secondary alcohol dehydrogenase (SADH), and a combination thereof.

The Cac may overexpress one or more enzymes selected from the group consisting of THL of Escherichia coli (NCBI Reference Sequence: WP_000786547.1), CTFA&B of Escherichia coli (NCBI Reference Sequence: WP_000850540.1 and WP_000339065.1) or Clostridium acetobutylicum (NCBI Reference Sequence: WP_010890847.1 and WP_010890848.1), SADH of Clostridium beijerinckii (NCBI Reference Sequence: WP_077844196.1), and a combination thereof.

The Clj may overexpress one or more enzymes selected from the group consisting of THL of Escherichia coli (NCBI Reference Sequence: WP_000786547.1), CTFA&B of Escherichia coli (NCBI Reference Sequence: WP_000850540.1 and WP_000339065.1) or Clostridium acetobutylicum (NCBI Reference Sequence: WP_010890847.1 and WP_010890848.1), SADH of Clostridium beijerinckii (NCBI Reference Sequence: WP_077844196.1), and a combination thereof.

The Cac may overexpress one or more enzymes selected from the group consisting of Acetoacetyl-CoA: acetate/butyrate CoA transferases (CTFA&B), thiolase (THL), acetoacetate decarboxylase (ADC), secondary alcohol dehydrogenase (SADH), or a combination thereof, and one or more enzymes selected from the group consisting of THL of Escherichia coli (NCBI Reference Sequence: WP_000786547.1), CTFA&B of Escherichia coli (NCBI Reference Sequence: WP_000850540.1 and WP_000339065.1) or Clostridium acetobutylicum (NCBI Reference Sequence: WP_010890847.1 and WP_010890848.1), SADH of Clostridium beijerinckii (NCBI Reference Sequence: WP_077844196.1), and a combination thereof.

The Clj may overexpress one or more enzymes selected from the group consisting of Acetoacetyl-CoA: acetate/butyrate CoA transferases (CTFA&B), thiolase

(THL), acetoacetate decarboxylase (ADC), secondary alcohol dehydrogenase (SADH), or a combination thereof, and one or more enzymes selected from the group consisting of THL of Escherichia coli (NCBI Reference Sequence: WP_000786547.1), CTFA&B of Escherichia coli (NCBI Reference Sequence: WP_000850540.1 and WP_000339065.1) or Clostridium acetobutylicum (NCBI Reference Sequence: WP_010890847.1 and WP_010890848.1), SADH of Clostridium beijerinckii (NCBI Reference Sequence: WP_077844196.1), and a combination thereof.

Where spCas9 from Streptococcus Pyogenes (NCBI Reference Sequence: WP_010922251.1) is integrated into a chromosome of the Cac at the location of the endogenous lactate dehydrogenase gene (ldhA, Locus tag: CA_C0267), the method may further comprise disrupting the function of the ldhA in Cac. One or more endogenous enzymes may be deleted from the chromosome or inactivated in the Cac, and the one or more endogenous enzymes may be selected from the group consisting of 3-hydroxybutyryl-CoA dehydrogenase gene (hbd, Locus tag: CA_C2708), short-chain-enoyl-CoA hydratase gene (crt, Locus tag: CA_C2712), acyl-CoA dehydrogenase gene (bcd, Locus tag: CA_C2711) and a combination thereof. Endogenous 3-hydroxybutyryl-CoA dehydrogenase gene (hbd, Locus tag: CA_C2708) may be deleted from the chromosome or inactivated in the Cac. Endogenous short-chain-enoyl-CoA hydratase gene (crt, Locus tag: CA_C2712) may be deleted from the chromosome or inactivated in the Cac. Endogenous acyl-CoA dehydrogenase gene (bcd, Locus tag: CA_C2711) may be deleted from the chromosome or inactivated in the Cac.

Where the medium comprises one or more exogenous substrates selected from the group consisting of butyrate, crotonate, 3-hydroxybutyrate, and a combination thereof, for example, at a concentration of about 0.1-2,000, 0.1-1,000, 0.1-500, 0.1-400, 0.1-300, 0.1-200, 0.1-100, 0.1-50, 0.1-10, 0.1-5, 0.1-1, 0.1-0.5, 0.5-2,000, 0.5-1,000, 0.5-500, 0.5-400, 0.5-300, 0.5-200, 0.5-100, 0.5-50, 0.5-10, 0.5-5, 0.5-1, 1-2,000, 1-1,000, 1-500, 1-400, 1-300, 1-200, 1-100, 1-50, 1-10, 1-5, 10-2,000, 10-1,000, 10-500, 10-400, 10-300, 10-200, 10-100, 10-50, 50-2,000, 50-1,000, 50-500, 50-400, 50-300, 50-200, 50-100, 100-2,000, 100-1,000, 100-500, 100-400, 100-300, 100-200, 100-150 or 150-200 mM, the method may further comprise enhancing the cell growth of the Cac.

Where the medium further comprises exogenous butyrate at a concentration of about 0.1-2,000, 0.1-1,000, 0.1-500, 0.1-400, 0.1-300, 0.1-200, 0.1-100, 0.1-50, 0.1-10, 0.1-5, 0.1-1, 0.1-0.5, 0.5-2,000, 0.5-1,000, 0.5-500, 0.5-400, 0.5-300, 0.5-200, 0.5-100, 0.5-50, 0.5-10, 0.5-5, 0.5-1, 1-2,000, 1-1,000, 1-500, 1-400, 1-300, 1-200, 1-100, 1-50, 1-10, 1-5, 10-2,000, 10-1,000, 10-500, 10-400, 10-300, 10-200, 10-100, 10-50, 50-2,000, 50-1,000, 50-500, 50-400, 50-300, 50-200, 50-100, 100-2,000, 100-1,000, 100-500, 100-400, 100-300, 100-200, 100-150 or 150-200 mM, the method may further comprise enhancing the cell growth of the Cac.

Where the medium further comprises exogenous crotonate at a concentration of about 0.1-2,000, 0.1-1,000, 0.1-500, 0.1-400, 0.1-300, 0.1-200, 0.1-100, 0.1-50, 0.1-10, 0.1-5, 0.1-1, 0.1-0.5, 0.5-2,000, 0.5-1,000, 0.5-500, 0.5-400, 0.5-300, 0.5-200, 0.5-100, 0.5-50, 0.5-10, 0.5-5, 0.5-1, 1-2,000, 1-1,000, 1-500, 1-400, 1-300, 1-200, 1-100, 1-50, 1-10, 1-5, 10-2,000, 10-1,000, 10-500, 10-400, 10-300, 10-200, 10-100, 10-50, 50-2,000, 50-1,000, 50-500, 50-400, 50-300, 50-200, 50-100, 100-2,000, 100-1,000, 100-500, 100-400, 100-300, 100-200, 100-150 or 150-200 mM, the method may further comprise enhancing the cell growth of the Cac.

Where the medium further comprises exogenous 3-hydroxybutyrate at a concentration of about 0.1-2,000, 0.1-1,000, 0.1-500, 0.1-400, 0.1-300, 0.1-200, 0.1-100, 0.1-50, 0.1-10, 0.1-5, 0.1-1, 0.1-0.5, 0.5-2,000, 0.5-1,000, 0.5-500, 0.5-400, 0.5-300, 0.5-200, 0.5-100, 0.5-50, 0.5-10, 0.5-5, 0.5-1, 1-2,000, 1-1,000, 1-500, 1-400, 1-300, 1-200, 1-100, 1-50, 1-10, 1-5, 10-2,000, 10-1,000, 10-500, 10-400, 10-300, 10-200, 10-100, 10-50, 50-2,000, 50-1,000, 50-500, 50-400, 50-300, 50-200, 50-100, 100-2,000, 100-1,000, 100-500, 100-400, 100-300, 100-200, 100-150 or 150-200 mM, the method may further comprise enhancing the cell growth of the Cac.

The method may further comprise passing the co-culture in a fresh medium without one or more substrates selected from the group consisting of butyrate, crotonate, 3-hydroxybutyrate, and a combination thereof over at least about 5, 10, 15, 20, 30, 40 or 50 subcultures, and enhancing the cell growth of the Cac.

The method may further comprise passing the co-culture in a fresh medium without butyrate over at least about 5, 10, 15, 20, 30, 40 or 50 subcultures, and enhancing the cell growth of the Cac.

Where the first microorganism expresses a first fusion protein comprising a first half of a split fluorescence HaloTag protein, the second microorganism expresses a second fusion protein comprising a second half of the split fluorescence HaloTag protein, and a fluorescence signal is generated upon contact of the first fusion protein with the second fusion proteins, the method may further comprise detecting a fluorescence signal in the first microorganism or the second microorganism, and the presence of the fluorescence signal indicates a transfer of the first fusion protein from the first microorganism into the second microorganism or a transfer of the second fusion protein from the second microorganism into the first microorganism. The method may further comprise determining the percentage of the first microorganism or the second microorganism showing the fluorescence signal. The percentage of the first microorganism or the second microorganism showing the fluorescence signal may reflect the frequency of transfer of the first fusion protein or the second fusion protein between the two microorganisms.

About 10-100%, 10-90%, 10-80%, 10-70%, 20-100%, 20-90%, 20-80%, 20-70%, 30-100%, 30-90%, 30-80%, 30-70%, 40-100%, 40-90%, 40-80%, 40-70%, 50-100%, 50-90%, 50-80%, 50-70%, 60-100%, 60-90%, 60-80% or 60-70% of the carbon atoms in the metabolites may be from the one or more consumed exogenous carbohydrates.

The co-culture may produce the metabolites with a product yield higher than about 0.01, 0.05, 0.1, 0.2, 0.3, 0.4, 0.5, 0.6, 0.7, 0.8, 0.9, 1, 2, 3, 4, 5, 6, 7, 8, 9 or 10, or about 0.01-10, 0.01-5, 0.01-1, 0.01-0.5, 0.01-0.1, 0.05-10, 0.05-5, 0.05-1, 0.05-0.5, 0.05-0.1, 0.1-10, 0.1-5, 0.1-1, 0.1-0.5, 0.5-10, 0.5-5 or 0.5-1 Cmol of the produced isopropanol per Cmol of the one or more consumed exogenous carbohydrates.

The co-culture may produce the metabolites with a product yield higher than about 0.01, 0.05, 0.1, 0.2, 0.3, 0.4, 0.5, 0.6, 0.7, 0.8, 0.9, 1, 2, 3, 4, 5, 6, 7, 8, 9 or 10, or about 0.01-10, 0.01-5, 0.01-1, 0.01-0.5, 0.01-0.1, 0.05-10, 0.05-5, 0.05-1, 0.05-0.5, 0.05-0.1, 0.1-10, 0.1-5, 0.1-1, 0.1-0.5, 0.5-10, 0.5-5 or 0.5-1 Cmol of the produced acetone per Cmol of the one or more consumed exogenous carbohydrates.

The co-culture may produce the metabolites with a product yield higher than about 0.01, 0.05, 0.1, 0.2, 0.3, 0.4, 0.5, 0.6, 0.67, 0.7, 0.8, 0.9, 1, 2, 3, 4, 5, 6, 7, 8, 9 or 10, or about 0.01-10, 0.01-5, 0.01-1, 0.01-0.5, 0.01-0.1, 0.05-10, 0.05-5, 0.05-1, 0.05-0.5, 0.05-0.1, 0.1-10, 0.1-5, 0.1-1, 0.1-0.5, 0.5-10, 0.5-5, 0.5-1, 0.5-0.9, 0.5-0.8, 0.5-0.7, 0.5-0.6, 0.6-10, 0.6-5, 0.6-1, 0.6-0.9, 0.6-0.8 or 0.6-0.7 Cmol of the produced ethanol per Cmol of the one or more consumed exogenous carbohydrates.

### Second Aspect of the Invention

According to the second aspect of the present invention, a method for producing metabolites by a co-culture is provided. The co-culture comprises a first microorganism and a second microorganism in a medium, and the first microorganism is different from the second microorganism. The method comprises: (a) growing the first microorganism and the second microorganism in the medium, wherein the medium comprises one or more exogenous carbohydrates, exogenous H₂, and exogenous CO₂; (b) consuming the one or more exogenous carbohydrates by the first microorganism, and producing acetone, first acetate, butyrate, butanol, CO₂ and H₂ by the first microorganism, wherein the first microorganism does not utilize for cell growth the exogenous CO₂ or exogenous H₂; (c) consuming the produced acetone, the produced CO₂, the produced H₂, the exogenous CO₂, and the exogenous H₂ by the second microorganism, and producing isopropanol, second acetate, and ethanol by the second microorganism, wherein the second microorganism does not produce CO₂ or H₂; (d) consuming the second produced acetate by the first microorganism, and producing acetyl-CoA by the first microorganism; and (e) forming cell membrane fusion by the first microorganism and the second microorganism, wherein electrons, the produced acetone, the produced CO₂ and the produced H₂ are transferred from the first microorganism into the second microorganism via the cell membrane fusion, and wherein the second produced acetate is transferred from the second microorganism into the first microorganism via the cell membrane fusion. The metabolites comprise the produced acetone, the produced isopropanol, the produced ethanol, the produced first acetate, the produced butyrate, the produced butanol, and the produced second acetate.

The recombinant microorganism may be derived from a microorganism selected from the group consisting of Clostridium acetobutylicum, Clostridium ljungdahlii, Eubacterium limosum, Clostridium beijerinckii, Clostridium autoethanogenum, Acetobacterium woodii, Clostridium saccharolyticum, Clostridium butyricum and Clostridium carboxydivorans. The recombinant microorganism may overexpress one or more enzymes selected from the group consisting of Acetoacetyl-CoA: acetate/butyrate CoA transferases (CTFA&B), thiolase (THL), acetoacetate decarboxylase (ADC), secondary alcohol dehydrogenase (SADH), and a combination thereof; and/or one or more enzymes selected from the group consisting of THL of Escherichia coli (NCBI Reference Sequence: WP_000786547.1), CTFA&B of Escherichia coli (NCBI Reference

Sequence: WP_000850540.1 and WP_000339065.1) or Clostridium acetobutylicum (NCBI Reference Sequence: WP_010890847.1 and WP_010890848.1), SADH of Clostridium beijerinckii (NCBI Reference Sequence: WP_077844196.1), and a combination thereof. The recombinant microorganism may be Clostridium acetobutylicum (Cac). spCas9 (NCBI Reference Sequence: WP_010922251.1) may be integrated into a chromosome of the recombinant microorganism at the location of endogenous lactate dehydrogenase gene (ldhA, Locus tag: CA_C0267). Endogenous 3-hydroxybutyryl-CoA dehydrogenase gene (hbd, Locus tag: CA_C2708) may be deleted from the chromosome in the recombinant microorganism. The recombinant microorganism may not utilize for cell growth exogenous CO₂ or exogenous H₂. In the co-culture for producing metabolites, including butanol, the recombinant microorganism may be Clostridium acetobutylicum (Cac), and the additional microorganism may be Clostridium ljungdahlii (Clj). The one or more exogenous carbohydrates may consist of glucose. The Cac may consume the glucose. The Clj may consume the exogenous CO₂. At least about 50%, 60%, 70%, 80% or 90%, or about 50-99%, 50-90%, 50-80%, 50-70%, 50-60%, 60-99%, 60-90%, 60-80%, 60-70%, 70-99%, 70-90%, 70-80%, 80-99%, or 80-90% of the carbon atoms in the consumed glucose may be transferred into the produced isopropanol. Less than about 40%, 30%, 25%, 20%, 10% or 5%, or about 0.1-40%, 0.1-30%, 0.1-25%, 0.1-20%, 0.1-10%, 0.1-5%, 0.1-1%, 0.1-0.5%, 0.2-40%, 0.2-30%, 0.2-25%, 0.2-20%, 0.2-10%, 0.2-5%, 0.2-1%, 0.2-0.5%, 0.5-40%, 0.5-30%, 0.5-25%, 0.5-20%, 0.5-10%, 0.5-5%, 0.5-1%, 1-40%, 1-30%, 1-25%, 1-20%, 1-10%, 1-5%, 5-40%, 5-30%, 5-25%, 5-20% or 5-10% of the carbon atoms in the consumed glucose may be transferred into the produced ethanol, the produced butanol, or a combination thereof. The molar ratio of the consumed glucose to the consumed exogenous CO₂ may be from about 10:1 to about 1:10, from about 2:1 to about 1:6, or from about 2:1 to about 1:2. About 1-95%, 1-90%, 1-80%, 1-70%, 5-95%, 5-90%, 5-80%, 5-70%, 10-95%, 10-90%, 10-80%, 10-70%, 20-95%, 20-90%, 20-80% or 20-70% of the carbon atoms in the metabolites for each mol of the consumed glucose may be from the consumed exogenous CO₂.

The co-culture may produce the metabolites with a product yield higher than about, 0.01, 0.05, 0.1, 0.2, 0.3, 0.4, 0.5, 0.6, 0.67, 0.7, 0.8, 0.9, 1, 2, 3, 4, 5, 6, 7, 8, 9 or 10, or about 0.01-10, 0.01-5, 0.01-1, 0.01-0.5, 0.01-0.1, 0.05-10, 0.05-5, 0.05-1, 0.05-0.5, 0.05-0.1, 0.1-10, 0.1-5, 0.1-1, 0.1-0.5, 0.5-10, 0.5-5, 0.5-1, 0.5-0.9, 0.5-0.8, 0.5-0.7, 0.5-0.6, 0.6-10, 0.6-5, 0.6-1, 0.6-0.9, 0.6-0.8 or 0.6-0.7 Cmol of a mixture of the produced isopropanol, the produced butyrate, the produced butanol, and the produced ethanol per Cmol of the one or more consumed exogenous carbohydrates.

The co-culture may produce the metabolites with a product yield higher than about, 0.01, 0.05, 0.1, 0.2, 0.3, 0.4, 0.5, 0.6, 0.67, 0.7, 0.8, 0.9, 1, 2, 3, 4, 5, 6, 7, 8, 9 or 10, or about 0.01-10, 0.01-5, 0.01-1, 0.01-0.5, 0.01-0.1, 0.05-10, 0.05-5, 0.05-1, 0.05-0.5, 0.05-0.1, 0.1-10, 0.1-5, 0.1-1, 0.1-0.5, 0.5-10, 0.5-5, 0.5-1, 0.5-0.9, 0.5-0.8, 0.5-0.7, 0.5-0.6, 0.6-10, 0.6-5, 0.6-1, 0.6-0.9, 0.6-0.8 or 0.6-0.7 Cmol of a mixture of the produced acetone, the produced butyrate, the produced butanol, and the produced ethanol per Cmol of the one or more consumed exogenous carbohydrates.

Third aspect of the invention According to the third aspect of the present invention, a recombinant microorganism is provided. The recombinant microorganism is derived from a microorganism selected from the group consisting of Clostridium acetobutylicum, Clostridium ljungdahlii, Eubacterium limosum, Clostridium beijerinckii, Clostridium autoethanogenum, Acetobacterium woodii, Clostridium saccharolyticum, Clostridium butyricum and Clostridium carboxydivorans. The recombinant microorganism overexpresses one or more enzymes selected from the group consisting of Acetoacetyl-CoA: acetate/butyrate CoA transferases (CTFA&B), thiolase (THL), acetoacetate decarboxylase (ADC), secondary alcohol dehydrogenase (SADH), and a combination thereof; and/or one or more enzymes selected from the group consisting of THL of Escherichia coli (NCBI Reference Sequence: WP_000786547.1), CTFA&B of Escherichia coli (NCBI Reference Sequence: WP_000850540.1 and WP_000339065.1) or Clostridium acetobutylicum (NCBI Reference Sequence: WP_010890847.1 and

WP_010890848.1), SADH of Clostridium beijerinckii (NCBI Reference Sequence: WP_077844196.1), and a combination thereof.

The recombinant microorganism may be Clostridium acetobutylicum (Cac). spCas9 (NCBI Reference Sequence: WP_010922251.1) may be integrated into a chromosome of the recombinant microorganism at the location of endogenous lactate dehydrogenase gene (ldhA, Locus tag: CA_C0267). Endogenous 3-hydroxybutyryl-CoA dehydrogenase gene (hbd, Locus tag: CA_C2708) may be deleted from the chromosome in the recombinant microorganism, and the recombinant microorganism may not produce butanol, butyrate, crotonate, butyraldehyde, beta-hydroxybutyrate or a long carbon chain length carboxylic acid. The recombinant microorganism may not utilize for cell growth exogenous CO₂ or exogenous H₂.

The recombinant microorganism may be Clostridium ljungdahlii (Clj). spCas9 (NCBI Reference Sequence: WP_010922251.1) may be integrated into a chromosome of the recombinant microorganism at the location of endogenous lactate dehydrogenase gene (ldhA, Locus tag: CA_C0267). Endogenous 3-hydroxybutyryl-CoA dehydrogenase gene (hbd, Locus tag: CA_C2708) may be deleted from the chromosome in the recombinant microorganism, and the recombinant microorganism may not produce butanol, butyrate, crotonate, butyraldehyde, beta-hydroxybutyrate or a long carbon chain length carboxylic acid. The recombinant microorganism may not utilize for cell growth exogenous CO₂ or exogenous H₂.

### Fourth Aspect of the Invention

According to the fourth aspect of the present invention, a co-culture for producing metabolites is provided. The co-culture comprises a recombinant microorganism and an additional microorganism in a medium. The medium comprises one or more exogenous carbohydrates, exogenous H₂, and exogenous CO₂. The recombinant microorganism is different from the additional microorganism. The recombinant microorganism consumes the one or more exogenous carbohydrates, and produces acetone, first acetate, butanol, CO₂ and H₂. The additional microorganism consumes the produced acetone, the produced CO₂, the produced H₂, the exogenous CO₂, and the exogenous H₂, and produces isopropanol, second acetate, and ethanol. The recombinant microorganism consumes the second produced acetate and produces acetyl-CoA. The recombinant microorganism and the additional microorganism form cell membrane fusion. Electrons, the produced acetone, the produced CO₂ and the produced H₂ are transferred from the recombinant microorganism into the additional microorganism via the cell membrane fusion. The second produced acetate is transferred from the additional microorganism into the recombinant microorganism via the cell membrane fusion. The metabolites comprise the produced acetone, the produced isopropanol, the produced ethanol, the produced first acetate, the produced butanol, and the produced second acetate.

The recombinant microorganism may be derived from a microorganism selected from the group consisting of Clostridium acetobutylicum, Clostridium ljungdahlii, Eubacterium limosum, Clostridium beijerinckii, Clostridium autoethanogenum, Acetobacterium woodii, Clostridium saccharolyticum, Clostridium butyricum and Clostridium carboxydivorans. The recombinant microorganism may overexpress one or more enzymes selected from the group consisting of Acetoacetyl-CoA: acetate/butyrate CoA transferases (CTFA&B), thiolase (THL), acetoacetate decarboxylase (ADC), secondary alcohol dehydrogenase (SADH), and a combination thereof; and/or one or more enzymes selected from the group consisting of THL of Escherichia coli (NCBI Reference Sequence: WP_000786547.1), CTFA&B of Escherichia coli (NCBI Reference Sequence: WP_000850540.1 and WP_000339065.1) or Clostridium acetobutylicum (NCBI Reference Sequence: WP_010890847.1 and WP_010890848.1), SADH of Clostridium beijerinckii (NCBI Reference Sequence: WP_077844196.1), and a combination thereof. The recombinant microorganism may be Clostridium acetobutylicum (Cac). spCas9 (NCBI Reference Sequence: WP_010922251.1) may be integrated into a chromosome of the recombinant microorganism at the location of endogenous lactate dehydrogenase gene (ldhA, Locus tag: CA_C0267). Endogenous 3-hydroxybutyryl-CoA dehydrogenase gene (hbd, Locus tag: CA_C2708) may be deleted from the chromosome in the recombinant microorganism. The recombinant microorganism may not utilize for cell growth exogenous CO₂ or exogenous H₂.

The recombinant microorganism may be Clostridium acetobutylicum (Cac), and the additional microorganism may be Clostridium ljungdahlii (Clj). The one or more exogenous carbohydrates may consist of glucose. The Cac may consume the glucose.

At least about 50%, 60%, 70%, 80% or 90%, or about 50-99%, 50-90%, 50-80%, 50-70%, 50-60%, 60-99%, 60-90%, 60-80%, 60-70%, 70-99%, 70-90%, 70-80%, 80-99%, or 80-90% of the carbon atoms in the consumed glucose may be transferred into the produced isopropanol. Less than about 40%, 30%, 25%, 20%, 10% or 5%, or about 0.1-40%, 0.1-30%, 0.1-25%, 0.1-20%, 0.1-10%, 0.1-5%, 0.1-1%, 0.1-0.5%, 0.2-40%, 0.2-30%, 0.2-25%, 0.2-20%, 0.2-10%, 0.2-5%, 0.2-1%, 0.2-0.5%, 0.5-40%, 0.5-30%, 0.5-25%, 0.5-20%, 0.5-10%, 0.5-5%, 0.5-1%, 1-40%, 1-30%, 1-25%, 1-20%, 1-10%, 1-5%, 5-40%, 5-30%, 5-25%, 5-20% or 5-10% of the carbon atoms in the consumed glucose may be transferred into the produced ethanol, the produced butanol, or a combination thereof. The molar ratio of the consumed glucose to the consumed exogenous CO₂ may be from about 10:1 to about 1:10, from about 2:1 to about 1:6, or from about 2:1 to about 1:2. About 1-95%, 1-90%, 1-80%, 1-70%, 5-95%, 5-90%, 5-80%, 5-70%, 10-95%, 10-90%, 10-80%, 10-70%, 20-95%, 20-90%, 20-80% or 20-70% of the carbon atoms in the metabolites for each mol of the consumed glucose may be from the consumed exogenous CO₂.

The medium may comprise one or more exogenous substrates selected from the group consisting of butyrate, crotonate, 3-hydroxybutyrate, and a combination thereof, for example, at a concentration of about 0.1-2,000, 0.1-1,000, 0.1-500, 0.1-400, 0.1-300, 0.1-200, 0.1-100, 0.1-50, 0.1-10, 0.1-5, 0.1-1, 0.1-0.5, 0.5-2,000, 0.5-1,000, 0.5-500, 0.5-400, 0.5-300, 0.5-200, 0.5-100, 0.5-50, 0.5-10, 0.5-5, 0.5-1, 1-2,000, 1-1,000, 1-500, 1-400, 1-300, 1-200, 1-100, 1-50, 1-10, 1-5, 10-2,000, 10-1,000, 10-500, 10-400, 10-300, 10-200, 10-100, 10-50, 50-2,000, 50-1,000, 50-500, 50-400, 50-300, 50-200, 50-100, 100-2,000, 100-1,000, 100-500, 100-400, 100-300, 100-200, 100-150 or 150-200 mM.

About 10-100%, 10-90%, 10-80%, 10-70%, 20-100%, 20-90%, 20-80%, 20-70%, 30-100%, 30-90%, 30-80%, 30-70%, 40-100%, 40-90%, 40-80%, 40-70%, 50-100%, 50-90%, 50-80%, 50-70%, 60-100%, 60-90%, 60-80% or 60-70% of the carbon atoms in the metabolites may be from the one or more consumed exogenous carbohydrates.

The metabolites may be produced by the co-culture with a product yield higher than about 0.01, 0.05, 0.1, 0.2, 0.3, 0.4, 0.5, 0.6, 0.7, 0.8, 0.9, 1, 2, 3, 4, 5, 6, 7, 8, 9 or 10, or about 0.01-10, 0.01-5, 0.01-1, 0.01-0.5, 0.01-0.1, 0.05-10, 0.05-5, 0.05-1, 0.05-0.5, 0.05-0.1, 0.1-10, 0.1-5, 0.1-1, 0.1-0.5, 0.5-10, 0.5-5 or 0.5-1 Cmol of the produced isopropanol per Cmol of the one or more consumed exogenous carbohydrates.

The metabolites may be produced by the co-culture with a product yield higher than about 0.01, 0.05, 0.1, 0.2, 0.3, 0.4, 0.5, 0.6, 0.7, 0.8, 0.9, 1, 2, 3, 4, 5, 6, 7, 8, 9 or 10, or about 0.01-10, 0.01-5, 0.01-1, 0.01-0.5, 0.01-0.1, 0.05-10, 0.05-5, 0.05-1, 0.05-0.5, 0.05-0.1, 0.1-10, 0.1-5, 0.1-1, 0.1-0.5, 0.5-10, 0.5-5 or 0.5-1 Cmol of the produced acetone per Cmol of the one or more consumed exogenous carbohydrates.

The metabolites may be produced by the co-culture with a product yield higher than about, 0.01, 0.05, 0.1, 0.2, 0.3, 0.4, 0.5, 0.6, 0.67, 0.7, 0.8, 0.9, 1, 2, 3, 4, 5, 6, 7, 8, 9 or 10, or about 0.01-10, 0.01-5, 0.01-1, 0.01-0.5, 0.01-0.1, 0.05-10, 0.05-5, 0.05-1, 0.05-0.5, 0.05-0.1, 0.1-10, 0.1-5, 0.1-1, 0.1-0.5, 0.5-10, 0.5-5, 0.5-1, 0.5-0.9, 0.5-0.8, 0.5-0.7, 0.5-0.6, 0.6-10, 0.6-5, 0.6-1, 0.6-0.9, 0.6-0.8 or 0.6-0.7 Cmol of the produced ethanol per Cmol of the one or more consumed exogenous carbohydrates.

### Fifth Aspect of the Invention

According to the fifth aspect of the present invention, a co-culture for producing metabolites is provided. The co-culture comprises a recombinant microorganism and an additional microorganism in a medium. The medium comprises one or more exogenous carbohydrates, exogenous H₂, and exogenous CO₂. The recombinant microorganism is different from the additional microorganism. The recombinant microorganism consumes the one or more exogenous carbohydrates, and produces acetone, first acetate, butyrate, CO₂ and H₂. The additional microorganism consumes the produced acetone, the produced CO₂, the produced H₂, the exogenous CO₂, and the exogenous H₂, and produces isopropanol, second acetate, and ethanol. The recombinant microorganism consumes the second produced acetate and produces acetyl-CoA. The recombinant microorganism and the additional microorganism form cell membrane fusion, wherein electrons, the produced acetone, the produced CO₂ and the produced H₂ are transferred from the recombinant microorganism into the additional microorganism via the cell membrane fusion, and wherein the second produced acetate is transferred from the additional microorganism into the recombinant microorganism via the cell membrane fusion. The metabolites comprise the produced acetone, the produced isopropanol, the produced ethanol, the produced first acetate, the produced butyrate, and the produced second acetate.

The recombinant microorganism may be derived from a microorganism selected from the group consisting of Clostridium acetobutylicum, Clostridium ljungdahlii, Eubacterium limosum, Clostridium beijerinckii, Clostridium autoethanogenum, Acetobacterium woodii, Clostridium saccharolyticum, Clostridium butyricum and Clostridium carboxydivorans. The recombinant microorganism may overexpress one or more enzymes selected from the group consisting of Acetoacetyl-CoA: acetate/butyrate CoA transferases (CTFA&B), thiolase (THL), acetoacetate decarboxylase (ADC), secondary alcohol dehydrogenase (SADH), and a combination thereof; and/or one or more enzymes selected from the group consisting of THL of Escherichia coli (NCBI Reference Sequence: WP_000786547.1), CTFA&B of Escherichia coli (NCBI Reference Sequence: WP_000850540.1 and WP_000339065.1) or Clostridium acetobutylicum

(NCBI Reference Sequence: WP_010890847.1 and WP_010890848.1), SADH of Clostridium beijerinckii (NCBI Reference Sequence: WP_077844196.1), and a combination thereof. The recombinant microorganism may be Clostridium acetobutylicum (Cac). spCas9 (NCBI Reference Sequence: WP_010922251.1) may be integrated into a chromosome of the recombinant microorganism at the location of endogenous lactate dehydrogenase gene (ldhA, Locus tag: CA_C0267). Endogenous 3-hydroxybutyryl-CoA dehydrogenase gene (hbd, Locus tag: CA_C2708) may be deleted from the chromosome in the recombinant microorganism. The recombinant microorganism may not utilize for cell growth exogenous CO₂ or exogenous H₂.

The recombinant microorganism may be Clostridium acetobutylicum (Cac), and the additional microorganism may be Clostridium ljungdahlii (Clj). The one or more exogenous carbohydrates may consist of glucose. The Cac may consume the glucose. The Clj may consume the exogenous CO₂. At least about 50%, 60%, 70%, 80% or 90%, or about 50-99%, 50-90%, 50-80%, 50-70%, 50-60%, 60-99%, 60-90%, 60-80%, 60-70%, 70-99%, 70-90%, 70-80%, 80-99%, or 80-90% of the carbon atoms in the consumed glucose may be transferred into the produced isopropanol. Less than about 40%, 30%, 25%, 20%, 10% or 5%, or about 0.1-40%, 0.1-30%, 0.1-25%, 0.1-20%, 0.1-10%, 0.1-5%, 0.1-1%, 0.1-0.5%, 0.2-40%, 0.2-30%, 0.2-25%, 0.2-20%, 0.2-10%, 0.2-5%, 0.2-1%, 0.2-0.5%, 0.5-40%, 0.5-30%, 0.5-25%, 0.5-20%, 0.5-10%, 0.5-5%, 0.5-1%, 1-40%, 1-30%, 1-25%, 1-20%, 1-10%, 1-5%, 5-40%, 5-30%, 5-25%, 5-20% or 5-10% of the carbon atoms in the consumed glucose may be transferred into the produced ethanol, the produced butanol, or a combination thereof. The molar ratio of the consumed glucose to the consumed exogenous CO₂ may be from about 10:1 to about 1:10, from about 2:1 to about 1:6, or from about 2:1 to about 1:2. About 1-95%, 1-90%, 1-80%, 1-70%, 5-95%, 5-90%, 5-80%, 5-70%, 10-95%, 10-90%, 10-80%, 10-70%, 20-95%, 20-90%, 20-80% or 20-70% of the carbon atoms in the metabolites for each mol of the consumed glucose may be from the consumed exogenous CO₂.

The co-culture may produce the metabolites with a product yield higher than about, 0.01, 0.05, 0.1, 0.2, 0.3, 0.4, 0.5, 0.6, 0.67, 0.7, 0.8, 0.9, 1, 2, 3, 4, 5, 6, 7, 8, 9 or 10, or about 0.01-10, 0.01-5, 0.01-1, 0.01-0.5, 0.01-0.1, 0.05-10, 0.05-5, 0.05-1, 0.05-0.5, 0.05-0.1, 0.1-10, 0.1-5, 0.1-1, 0.1-0.5, 0.5-10, 0.5-5, 0.5-1, 0.5-0.9, 0.5-0.8, 0.5-0.7, 0.5-0.6, 0.6-10, 0.6-5, 0.6-1, 0.6-0.9, 0.6-0.8 or 0.6-0.7 Cmol of a mixture of the produced isopropanol, the produced butyrate, and the produced ethanol per Cmol of the one or more consumed exogenous carbohydrates.

The co-culture may produce the metabolites with a product yield higher than about, 0.01, 0.05, 0.1, 0.2, 0.3, 0.4, 0.5, 0.6, 0.67, 0.7, 0.8, 0.9, 1, 2, 3, 4, 5, 6, 7, 8, 9 or 10, or about 0.01-10, 0.01-5, 0.01-1, 0.01-0.5, 0.01-0.1, 0.05-10, 0.05-5, 0.05-1, 0.05-0.5, 0.05-0.1, 0.1-10, 0.1-5, 0.1-1, 0.1-0.5, 0.5-10, 0.5-5, 0.5-1, 0.5-0.9, 0.5-0.8, 0.5-0.7, 0.5-0.6, 0.6-10, 0.6-5, 0.6-1, 0.6-0.9, 0.6-0.8 or 0.6-0.7 Cmol of a mixture of the produced acetone, the produced butyrate, and the produced ethanol per

Cmol of the one or more consumed exogenous carbohydrates.

The term “about” as used herein when referring to a measurable value such as an amount, a percentage, and the like, is meant to encompass variations of +20% or +10%, more preferably +5%, even more preferably +1%, and still more preferably +0.1% from the specified value, as such variations are appropriate.

### Example 1. Construction of Plasmids for Acetone Production in Asporogenous Cac M5 Strain

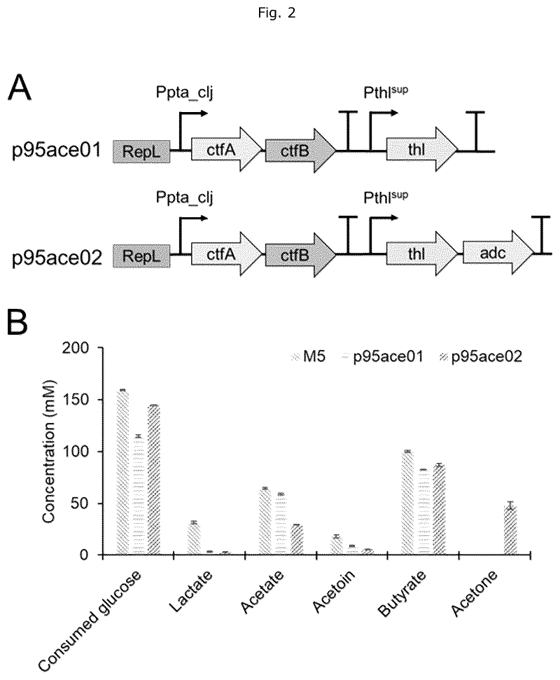

Acetone production pathway genes (i.e., ctfAB, thl, and adc) were cloned to a Cac-E. coli shuttle vector plasmid under the control of strong and constitutive promoters: pta_clj, and this up. Decarboxylation of acetoacetate to acetone is a thermodynamically favorable reaction; it is thus possible that adc expression is not necessary. To test this hypothesis, plasmid harboring ctfAB and thl without adc (p95ace01a), and with adc (p95ace02a) were constructed (FIG. 2A). The constructs were verified by PCR, restriction enzyme, and Sanger sequencing. The constructed p95ace01a and p95ace02a plasmids were introduced to Cac M5 and tested for acetone production. For rapid characterization, we cultured the engineered Cac M5 in CGM glucose medium without pH control (FIG. 2B). Cac M5 with p95ace02 produced 47 mM acetone from 144 mM consumed glucose, while p95ace01a produced no detectable amount of acetone. The engineered M5 strains produced less pyruvate derived metabolites (i.e., lactate and acetoin) than the parental M5 strain.

### Example 2. Engineering Translation Initiation Rate of Acetone Pathway Genes Through RBS Engineering

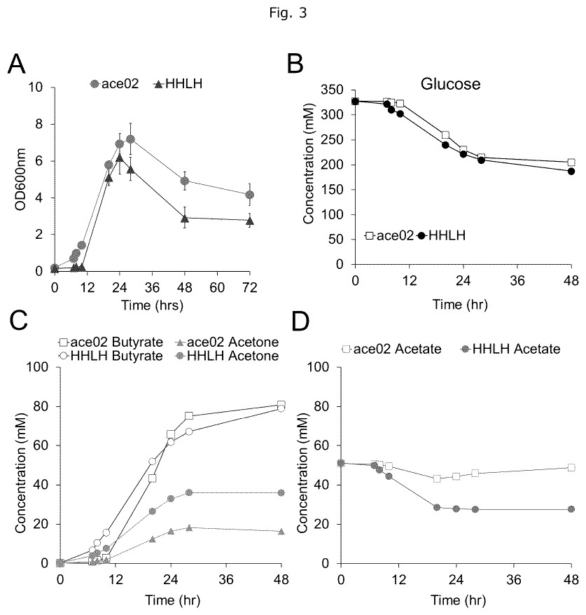

Our initial acetone production plasmid construct (p95ace02a) containing the four acetone production pathway genes, the CoA transferase genes, ctfAB; thiolase, thl; and acetoacetate decarboxylase, adc, was further engineered to maximize the gene expression level. Since we already deployed the two strongest constitutive promoters (i.e., Ppta_clj and Pthi^{sup}), ribosome binding site (RBS) engineering was pursued to improve translation initiation rates of the acetone biosynthesis genes. This strategy combined the strong constitutive promoters with synthetic RBSs for acetone biosynthesis gene expression in Cac. Synthetic RBS and 5′ untranslated region (UTR) sequences for ctfA, ctfB, and adc were individually designed in silico using the RBS calculator (https://salislab.net/software/). The RBS engineered acetone production plasmids were successfully constructed, and sequence was verified. Although thl is one of the acetone biosynthesis genes, we did not engineer the RBS of thl at this point because the endogenous thiolase activity was strong enough, producing a high amount of butyrate. The plasmid with maximized translation rates of ctfA, ctfB, and adc was named as p95ace02a_HHLH. The rationally designed p95ace02a_HHLH is a gene expression plasmid for very strong acetone biosynthesis in Cac. We transformed Cac M5 strain with p95ace02a_HHLH plasmid to test acetone production. For rapid characterization, we cultured the engineered Cac M5 in CGM glucose medium without pH control. We monitored glucose consumption and major metabolite formation for 48 hours culture. As a control, Cac M5 harboring p95ace02a (non-RBS engineered version) was cultured under the same condition to determine the effect of the RBS engineering (FIG. 3). The Cac M5 strains harboring p95ace02a and p95ace02a_HHLH were named as ace02 and HHLH, respectively (FIG. 3). Cell growth rate and maximum cell density of HHLH was slightly lower than ace02, while glucose consumption of the two strains was similar. The HHLH strain produced 2.1-fold higher acetone titers (41 mM) with a 1.8-fold higher volumetric production rate (1.8 mM/h) compared to the ace02 strain, which produced acetone at the titers and production rate of 19 mM and 1.0 mM/h, respectively. Interestingly, the HHLH consumed 2.9-fold more acetate (23 mM) compared to ace02 strain (8 mM). The results suggest that the p95ace02a_HHLH plasmid expressed the acetone biosynthesis genes better than the control p95ace02a plasmid.

### Example 3. Acetone Production by Cac 824 Strain Harboring the p95Ace02a Plasmid

Monoculture of Cac harboring the best plasmid (p95ace02a) was characterized in a mini-bioreactor under pH 5.5. The results and conclusions were summarized in Table 1. We explored the effect of initial inoculum size and nutrient feeding strategy by varying initial OD (Std OD at ˜0.1, 3×OD at ˜0.3) and feeding strategy (i.e., 120% or 220% of the normal nutrient levels consisting of yeast extract, vitamins, and minerals) to ensure that the cells were not nutrient-limited. The acetone titer and productivity reached 9.0 g/L and 0.52 g/L/h, respectively. Our goal is to achieve 0.86 g/L/h at 1L batch culture with at least 0.24 g/g yield.

TABLE 1
A summary of acetone production in a pH-controlled bioreactor.

| | | | | | Acetone | | | |
|---|---|---|---|---|---|---|---|---|
| | | | | Acetone | Peak | | | |
| | | | | Peak | Titer | Maximum | Time | |
| | | | | Titer | (g/L, | Rate, | to | |
| | Cac | Clj | Special | (mM, for | for | Acetone | Titer | |
| ID | Spec. | Spec. | Cond.'s | monoc.) | monoc.) | (g/L-hr) | (hr) | Conclusions |
| F1− | 824 | mono | Std | 148 | 8.6 | 0.49 | 32 | The standard |
| | (p95ace02a) | | OD, | | | | | OD leads to |
| | | | 120% | | | | | the highest |
| | | | nutr | | | | | peak acetone |
| F1+ | 824 | mono | Std | 155 | 9.0 | 0.44 | 32 | titers. |
| | (p95ace02a) | | OD, | | | | | Additional |
| | | | 220% | | | | | nutrients |
| | | | nutr | | | | | don't |
| | | | | | | | | meaningfully |
| | | | | | | | | improve |
| | | | | | | | | acetone |
| | | | | | | | | production. |
| F3− | 824 | mono | 3x OD, | 139 | 8.1 | 0.52 | 32 | The |
| | (p95ace02a) | | 120% | | | | | increased OD |
| | | | nutr | | | | | slightly |
| F3+ | 824 | mono | 3x OD, | 141 | 8.2 | 0.52 | 32 | increases the |
| | (p95ace02a) | | 220% | | | | | maximum |
| | | | nutr | | | | | rate of |
| | | | | | | | | acetone |
| | | | | | | | | production, |
| | | | | | | | | but not in |
| | | | | | | | | proportion to |
| | | | | | | | | the increased |
| | | | | | | | | initial cell |
| | | | | | | | | density. |
| | | | | | | | | Additional |
| | | | | | | | | nutrients |
| | | | | | | | | don't |
| | | | | | | | | meaningfully |
| | | | | | | | | improve |
| | | | | | | | | production. |

### Example 4. Improved Acetone Production from C. acetobutylicum by Expressing a Heterologous THL from Escherichia coli

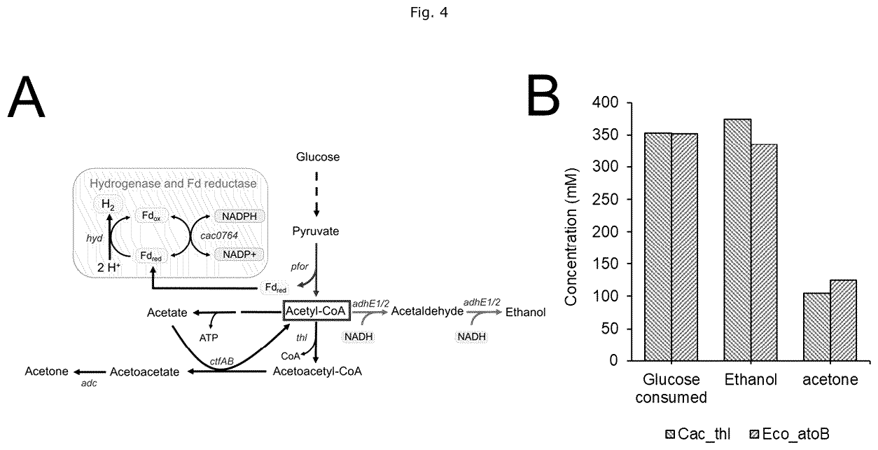

We hypothesized that the high ethanol production is due to the relatively weak thiolase activity compared to aldehyde/alcohol dehydrogenase activity (FIG. 4A). Since thiolase protein of Cac is redox-sensitive, acetone production could be inhibited by the significantly altered cellular redox state of CACas9 Δhbd. Whereby CACas9 contains a chromosomally integrated, xylose inducible Cas9 cassette at the lactate dehydrogenase (ldhA) location of WT Cac (See Example 12). Using the integrated Cas9, we deleted 3-hydroxybutyryl-CoA dehydrogenase (hbd) gene, disrupting the 4C metabolism (See Example 13). The disruption in 4C metabolism is the cause of the altered redox state. Therefore, we deployed a heterologous thiolase (encoded as atoB) from Escherichia coli that is insensitive to redox and catalytically more efficient than the endogenous Cac thiolase. We substituted the thl from p95ace02a with atoB, constructing a plasmid p95ace02atoB. Then, we compared acetone production by CACas9 Δhbd/p95ace02a and CACas9 Δhbd/p95ace02atoB (FIG. 4B). Acetone production titers from CACas9 Δhbd/p95ace02atoB monoculture reached up to 125 mM, which was improved by 19% as compared to CACas9 Δhbd/p95ace02a monoculture. As a trade-off, ethanol production was reduced by 11%, proving our hypothesis that pulling the flux towards acetoacetyl-CoA would improve acetone production and reduce ethanol production. However, we still observed the major metabolic flux towards ethanol, likely due to electron management by Cac. It should be noted that ethanol production is electron balanced with glycolysis, and sufficient conversion of reduced ferredoxin into hydrogen is required to drive the flux towards acetone over ethanol (FIG. 4A).

### Example 5. Different Cellular and Metabolic Behaviors of the Coculture Comprising Acetone Overproducing C. acetobutylicum and C. ljungdahlii Compared to the Monoculture of the C. acetobutylicum

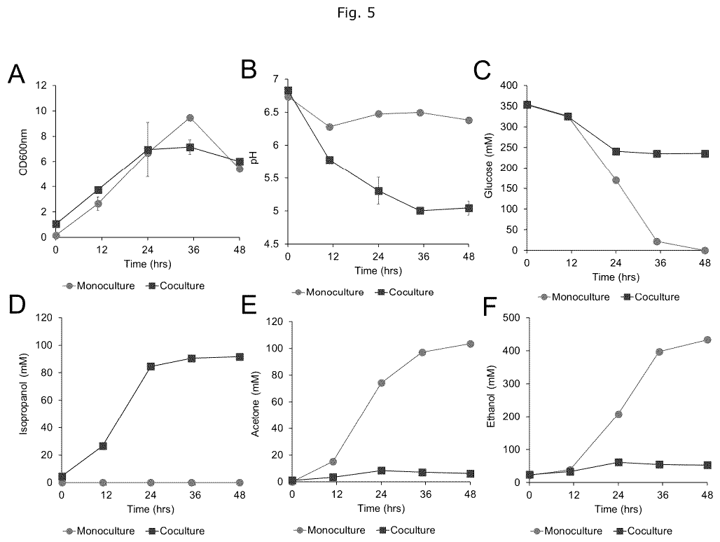

To improve acetone production, we transformed CACas9 Δhbd with p95ace02a, a plasmid harboring acetone production pathway gene (i.e., ctfAB, thl, and adc) under the control of strong constitutive promoters. CACas9 Δhbd/p95ace02a strain was used in monoculture or co-culture with Clj in serum bottles (working volume of 20 mL) without pH adjustment. The monoculture cell growth reached to typical optical density (OD) level of 9.5, suggesting that CACas9 Δhbd/p95ace02a retained robust glucose fermentation capability (FIG. 5A). Coculture of CACas9 Δhbd/p95ace02a and Clj showed a lower than typical OD level around 7.0. Interestingly, monoculture of CACas9 Δhbd/p95ace02a maintained pH level at between 6.0 and 6.5, completing glucose consumption without pH adjustment (FIG. 5B, C). Only 14 mM of acetate were left after 48 hours, suggesting that CACas9 Δhbd/p95ace02a assimilated (re-utilized) most of the produced acetate for acetone production. Because CACas9 Δhbd/p95ace02a did not produce butyrate, acetate was the major organic acid product. Therefore, the acetate assimilation might cause the pH increase as observed in 7B. In contrast, coculture of CACas9 Δhbd/p95ace02a and Clj lowered the pH down to 5.0 and accumulated acetate up to 143 mM. Surprisingly, glucose consumption of CACas9 Δhbd/p95ace02a was significantly inhibited after 24 hours from the coculture (FIG. 5C). The inhibited glucose consumption caused suboptimal isopropanol (IPA) production up to the titers of 90 mM (FIG. 5D). CACas9 Δhbd/p95ace02a monoculture produced 103 mM of acetone without such notable inhibition. As expected, CACas9 Δhbd/p95ace02a monoculture produced high level of ethanol up to 409 mM to compensate for the loss of electron sinks in the deleted 4-C pathway (FIG. 5F). Interestingly, ethanol titers (35 mM) from the coculture were significantly lower compared to the monoculture. Due to the low ethanol titers, isopropanol/ethanol ratio reached to 2.5 (mol/mol). The results show that optimizing the coculture can achieve the targeted high isopropanol titers, yields, and alcohol selectivity.

### Example 6. Direct Contact with Clj Halts Glycolysis after 24 Hours in Cac Δhbd p95Ace02a

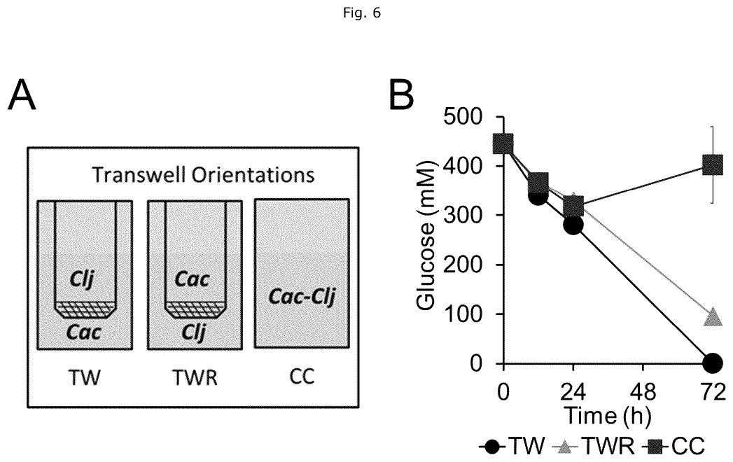

During characterization of CACas9 Δhbd, we observed that coculture fermentations of Clj with CACas9 Δhbd expressing the p95ace02a acetone plasmid would halt around 24 hours, consuming less than 50% of the available glucose. This issue was not observed in monoculture Cac fermentations. To examine the mechanism by which the presence of Clj inhibits full glucose utilization, we performed a transwell experiment in which cells were separated by a permeable membrane. We hypothesized that, if Clj inhibited Cac via exchange of metabolites or proteins through the liquid medium, glucose utilization would be incomplete both when the cells were separated by the membrane and when they were mixed together. However, if Clj inhibited Cac via direct cell to cell contact, the mixed control would fail to utilize all available glucose, similar to the serum bottles, but the membrane separated controls would utilize most or all of the available glucose (FIG. 6).

We sampled the cultures at 12, 24, and 72 hours. In the serum bottle cocultures the cells consumed glucose at a normal rate up to approximately 24 hours, after which glucose utilization ceased. This was the pattern we observed in the mixed controls in our transwell experiments. However, all wells with a membrane separating the two species consumed most or all the glucose after 72 hours. A repeated experiment with 2-fold higher initial concentration of cells of both species showed the same pattern. The results show that direct contact with Clj is indeed responsible for halting glycolysis in Cac after 24 hours of fermentation.

### Example 7. Improving Robustness of the Coculture Comprising C. acetobutylicum and C. ljungdahlii

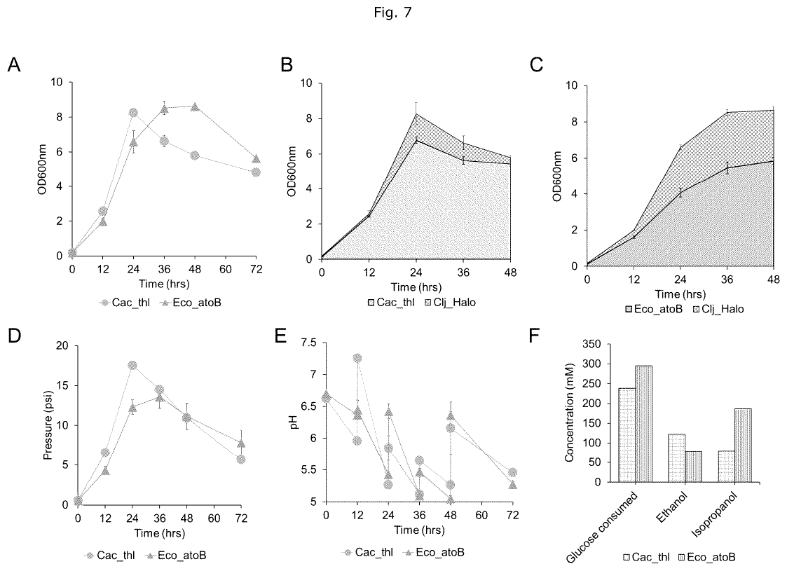

Repetitive coculture experiments employing CACas9 Δhbd/p95ace02a revealed that the strain stopped the glucose consumption around 24 hours when cocultured with Clj. To interrogate the problem, we analyzed population dynamics of Cac-Clj cocultures by expressing a far-red fluorescence Halotag protein in Clj (Clj/p100ptaHalo, Clj_Halo). The Halotag expressing Clj is efficiently fluorescence-labeled (>98%) upon addition of ligand in a coculture sample. CACas9 Δhbd/p95ace02a (Cac_thl) and CACas9 Δhbd/p95ace02atoB (Eco_atoB) strains were cocultured with Clj/p100ptaHalo in serum bottles with intermittent pH adjustment. The two cocultures showed similar OD up to 24 hours but significantly different patterns afterward (FIG. 7A). Population analysis of the coculture samples revealed that the coculture of Eco_atoB and Clj_Halo was more robust compared to the coculture of Cac_thl and Clj_Halo (FIGS. 7B and 7C). Cac_thl and Clj_Halo population declined after 24 hours, while Eco_atoB and Clj_Halo population consistently increased for 36 hours and maintained up to 48 hours. The surprising results clearly show that thiolase played some important roles in CACas9 Δhbd metabolism under the coculture condition, also affecting the cell growth of Clj.

Serum bottle pressure increased until the first 24 hours and then sharply declined from the coculture of Cac_thl and Clj_Halo (FIG. 7D), corresponding to the cell growth patterns. However, pH of the coculture constantly decreased for the next 48 hours, suggesting that Clj_Halo metabolism remained active, producing acetate from CO₂ and H₂ gas (FIG. 7E). Most importantly, the coculture of Eco_atoB and Clj_Halo produced isopropanol up to 187 mM, which was 2.4-fold higher titers compared to the coculture of Cac_thl and Clj_Halo (FIG. 7F).

Taken altogether, a newly constructed plasmid expressing the redox-insensitive thiolase from E. coli (p95ace02atoB) was beneficial not only for robust coculture, but also for efficient isopropanol production.

### Example 8. Improved Isopropanol Production by Overexpressing a Secondary Alcohol Dehydrogenase (SADH) Originated from C. beijerinckii (sadH_cbei) in C. acetobutylicum

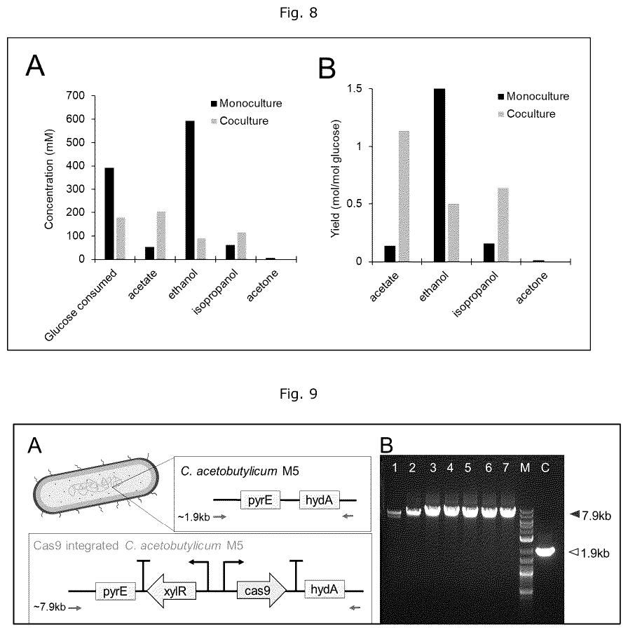

To reduce the ethanol byproduct formation and improve isopropanol production, we constructed a new plasmid derived from p95ace02atoB. We introduced a secondary alcohol dehydrogenase (sadh) from C. beijerinckii to plasmid p95ace02atoB, generating p95ace02atoB_sadh_cbei. Coculture of CACas9 Δhbd containing the p95ace02atoB_sadh_cbei with Clj/p100ptaHalo further improved isopropanol yields up to 0.64 (mol/mol glucose), significantly improving the titers and product selectivity (FIG. 8).

### Example 9. SpCas9 Integration into the Cac M5 Chromosome

For rapid strain development, CRISPR/Cas9 technology was applied to C. acetobutylicum. A xylose-inducible promoter and regulator from C. difficile is tight enough to minimize toxicity of Cas9 and strong enough to express Cas9 from chromosome. We integrated Cas9 into Cac M5 chromosome (FIG. 9A). Cac M5 is missing the pSOL1 megaplasmid and as a result it does not sporulate and does not produce solvents, unlike the parent WT Cac strain. We confirmed the integration by

PCR using primers specifically binding to upstream and downstream of the Cas9 integration site (FIG. 9B). The correct integration location was further verified by Sanger sequencing. After curing the Cas9 integration plasmid by several passages on medium without antibiotics, a single colony was isolated, and the strain was named M5Cas9. A sgRNA expressing plasmid harboring homology arms can be introduced to M5Cas9 followed by xylose induced Cas9 expression for genome editing.

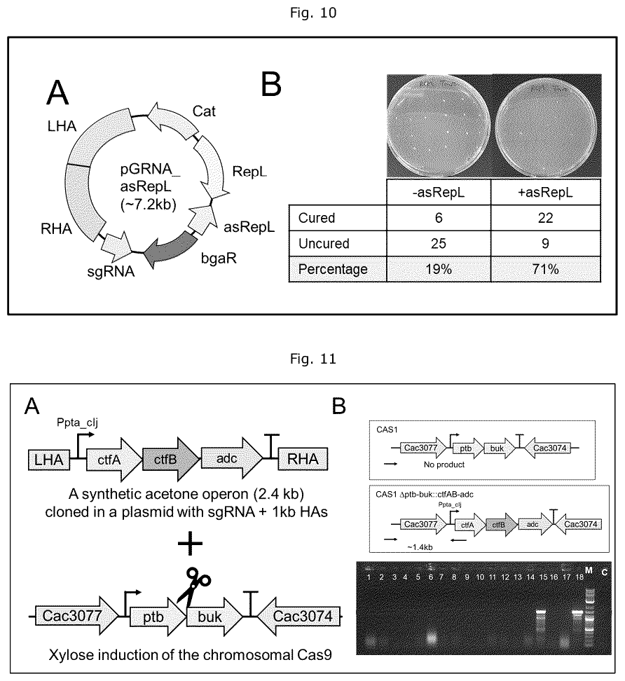

The M5Cas9 can be transformed with a sgRNA expression plasmid (pGRNA) that includes sgRNA, scaffolds, and homology arms to guide the chromosomally expressed Cas9 and edit a targeted location with homologous recombination. Plasmids in Cac are relatively stable even without antibiotics and therefore, plasmid curing after editing is time consuming. To accelerate the plasmid curing, we modified pGRNA with lactose inducible antisense RNA against its origin of replication (asRepL), named pGRNA_asRepL (FIG. 10A). Transformation efficiency of M5Cas9 strain with pGRNA_asRepL achieved ˜1×10³ CFU/micro-gram of DNA, which was comparable to the previously reported efficiency with pGRNA (˜5×10³ CFU/micro-gram of DNA). The antisense RNA associated counterselection improved plasmid curing rate by 3.5-folds as compared to pGRNA without asRepL, accelerating the overall genome editing process (FIG. 10B).

### Example 10. Integration of a Synthetic Acetone Production Operon into the Chromosome of Cac

Robust chromosomal expression of the acetone pathway in Cac is essential for stable antibiotics-free isopropanol production. Expression of the full acetone pathway genes from the Cac chromosome has not been achieved for lack of suitable genetic tools and strategy. To this effect, we constructed a plasmid containing a synthetic acetone-production operon (ctfA/B and adc genes) under the control of a strong pta_clj promoter with 1 kb homology arms for insertion at the ptb-buk location (FIG. 11A). After transformation and xylose induction, successful genome integration of the operon was confirmed by colony PCR using primers specific to the pta_clj promoter and the upstream region of ptb-buk site (FIG. 11B). For a rapid validation of genome engineering capability, we employed a published Cac WT based strain (named CAS1) having the integrated Cas9 cassette. Strain CAS1 includes deletion of a gene CA_C1502 coding for a restriction endonuclease, whose impact on cell metabolism has not been explored. Because CAS1 strain behaved differently from Cac WT in simple bottle cultures, we will carefully characterize the engineered strain while constructing a Cac strain without the CA_C1502 deletion. Overall, we successfully demonstrated integration of a synthetic 2.4 kb length acetone operon into the Cac chromosome.

### Example 11. Identification of Unpredicted Cellular Phenotype by the Chromosomal Integration of a Xylose Inducible Cas9 Expression Genetic Element

As described above, we successfully integrated a synthetic acetone production operon (Ppta_clj::ctfAB::adc) at ptb-buk location from the chromosome of Cac WT (CAS1 Δptb-buk::ACE). We characterized the strain with appropriate control strains including the parental CAS1 strain (Cac WT strain with xyIR-cas9 cassette chromosomally integrated at the upstream of hydA gene). We used our established bioreactor conditions, including a pH setpoint of 5.5, nitrogen headspace gassing, and batch glucose feeding (Table 2). The results showed that the chromosomal integration of the synthetic acetone operon enhanced acetone production compared to the control strain (CAS1 Δptb-buk). However, the CAS1 strain showed significantly impaired acetone production, suggesting that the CAS1 is not a good base strain for acetone production. This shows the location of integration is very important and also that one cannot predict a priori what integration location is good. One skilled in the art could not have predicted that this integration would be harmful to the phenotypic performance. We concluded that the chromosomally integrated xyIR-cas9 at the upstream of the hydrogenase attenuated the solventogenesis as acetone production is metabolically related to hydrogen production. Therefore, we decided not to use the CAS1 strain for further engineering. We were able to identify another location to integrate the Cas9 gene without causing harm to the desirable phenotypic response.

TABLE 2
Acetone production by CAS1 based strain vs. WT Cac.

| | | | Acetone | Acetone | | | |
|---|---|---|---|---|---|---|---|
| | | | Peak | Peak | | | |
| | | | Titer | Titer | Maximum | Time | |
| | | | (mM, | (g/L, | Rate, | to | |
| | | Clj | for | for | Acetone | Titer | |
| ID | Cac Spec. | Spec. | monoc.) | monoc.) | (g/L-hr) | (hr) | Conclusions |
| H1 | CAS1 | mono | 0 | 0 | 0 | 45 | xyIR-cas9 |
| | | | | | | | integration site |
| | | | | | | | attenuated |
| | | | | | | | solventogenesis |
| | | | | | | | and acetone |
| | | | | | | | production. |
| H2 | CAS1 Δptb- | mono | 15 | 0.87 | 0.026 | 45 | ptb-buk deletion |
| | buk | | | | | | activated |
| | | | | | | | solventogenesis |
| | | | | | | | of CAS1 |
| H3 | CAS1 Δptb- | mono | 48 | 2.79 | 0.084 | 45 | The |
| | buk::ACE | | | | | | chromosomally |
| | | | | | | | integrated ACE |
| | | | | | | | operon enhanced |
| | | | | | | | acetone |
| | | | | | | | production. |
| H4 | CAS1 Δptb- | mono | 135 | 7.84 | 0.24 | 45 | The p95ace02a |
| | buk::ACE/ | | | | | | plasmid, our best |
| | p95ace02a | | | | | | acetone |
| | | | | | | | production |
| | | | | | | | plasmid, leads to |
| | | | | | | | the highest |
| | | | | | | | acetone |
| | | | | | | | production, but |
| | | | | | | | not as high as |
| | | | | | | | 824/p95ace02a. |
| J1 | 824 (WT) | mono | 122 | 7.1 | 0.33 | 51 | This is our WT |
| | | | | | | | benchmark going |
| | | | | | | | forward. |
| F1− | 824 | mono | 148 | 8.6 | 0.49 | 32 | p95ace02a |
| | (p95ace02a) | | | | | | plasmid improved |
| | | | | | | | acetone |
| | | | | | | | production. This |
| | | | | | | | is a performance |
| | | | | | | | from Q4 report. |

### Example 12. Integrating the xyIR-Cas9 Cassette at the ldhA Gene Location for Gene Editing

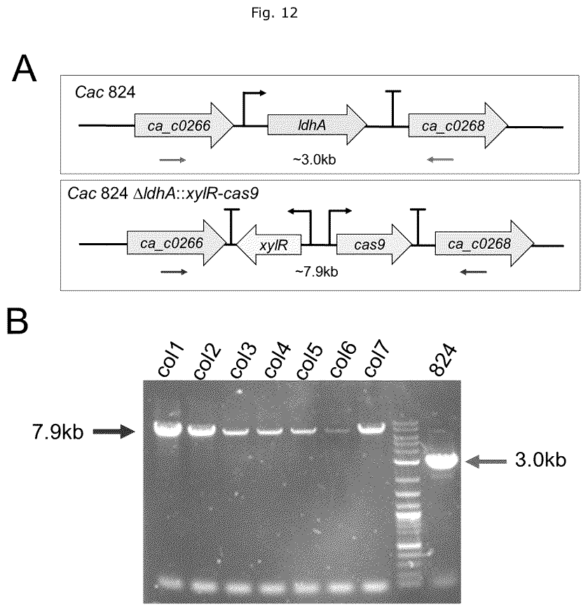

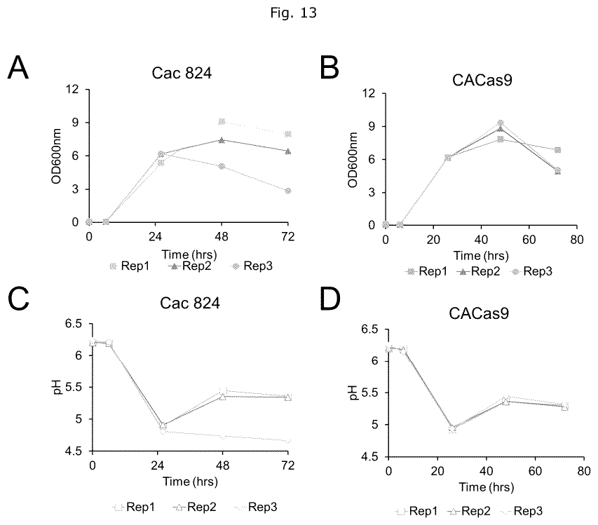

We changed the xyIR-cas9 cassette integration site to eliminate the polar effects of the original integration location near the hydA gene. We successfully integrated the xyIR-cas9 cassette into lactate dehydrogenase (ldhA) gene location that results in complete deletion of ldhA responsible for the undesired lactate formation (FIG. 12). The strain is designated as CACas9 that was used as the main chassis cell for genome engineering as well as acetone production. The non-naturally occurring C. acetobuylicum containing the xylose inducible cas9 cassette at ldhA location shows similar cell behaviors with the wild type C. acetobutylicum ATCC824, unlike CAS1 (FIG. 13, Table 2). Therefore, this is an important inventive step since one could not have predicted what location would NOT cause harmful effects on the phenotype of producing the desirable products, as it in the location of the CAS1 strain.

### Example 13. Deleting Hbd Gene from WT Cac Strain Using CRISPR/Cas9 and Adding Butyrate or Crotonate or Beta-Hydroxybutyrate to Enable Robust Cell Growth

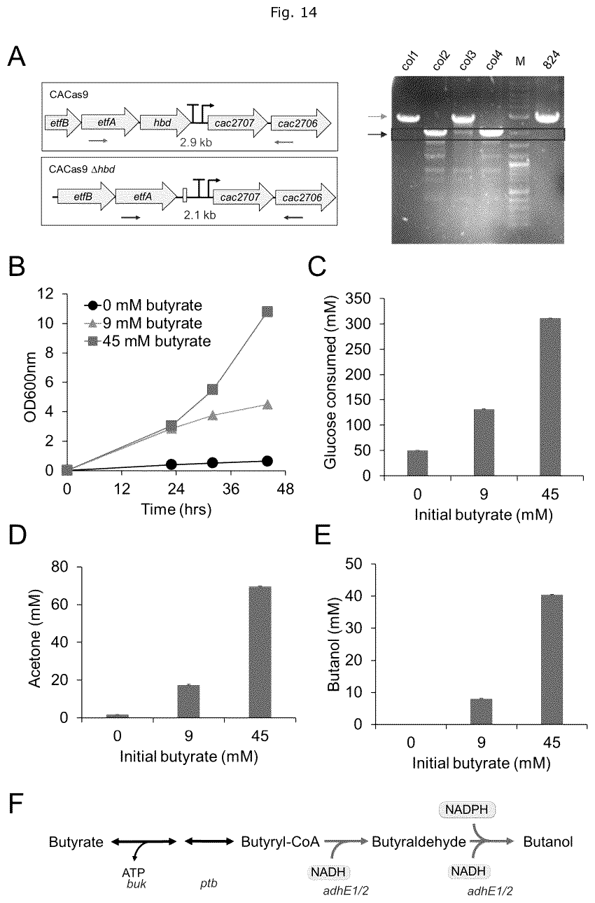

As described above, we chromosomally integrated the xylose inducible Cas9 cassette at lactate dehydrogenase (ldhA) location of WT Cac (we call this strain CACas9). Using the chromosomal Cas9 tool, we deleted 3-hydroxybutyryl-CoA dehydrogenase (hbd) gene, disrupting the 4C metabolism (FIG. 14A). CACas9 Δhbd strain showed significantly slower cell growth. We countered this poor growth by adding butyrate in the medium (FIG. 14B), thus largely restoring the growth of cells to those of the parent cells. The improved cell growth corresponded to enhanced glucose consumption (FIG. 14C) and acetone production (FIG. 14D). The ability of added butyrate to restore good growth to the hbd-deleted Cac strain was unexpected and unanticipated and an important inventive step. The supplemented/added butyrate was converted to butanol at >90% conversion ratio (FIG. 14E), likely through butyrate kinase (buk), phosphate butyryltransferase (ptb), and aldehyde/alcohol dehydrogenases (adhE) (FIG. 14F). Based on these results, we added 30 mM of butyrate in media for cultures of the hbd deleted Cac strains. The mechanism by which butyrate addition restores good cell growth to hbd-deleted Cac strains is regulatory in nature by differential activation of regulatory elements.

We also tested the impact of addition of crotonate or beta hydroxybutyrate instead of butyrate to enhance cell growth. We expressed the acetone production pathway genes (i.e., ctfAB, thl, and adc) in the CACas9 Δhbd strain supplemented crotonate or beta hydroxybutyrate to the medium at the concentration from 0 mM to 100 mM. Interestingly, the cell growth was significantly improved with shorter lag phase and the higher maximum OD when higher concentrations of crotonate or beta hydroxybutyrate were added to the medium. Cell densities were 6-8 times compared to cultures without the additions. The higher Lactate yield (mol/mol glucose) was reduced from 1.6, corresponding to 81% of theoretical maximum yield (2 moles of lactate from 1 mole of glucose) to 0.9 when 100 mM crotonate or beta hydroxybutyrate was added to the medium. The better electron management resulted in improved, 8-foldhigherm H₂ production. This suggests that the crotonate or beta hydroxybutyrate additions alleviated the accumulation of NADH, leading to less lactate production and more hydrogen production through hydrogenase. Finally, acetone production was increased by 11-fold by the crotonate or beta hydroxybutyrate additions. The results suggested that it would be important to reduce the accumulation of NADH generated from the glycolysis for robust cell growth of the hbd deleted strains and acetone production.

### Example 14. Adaptive Laboratory Evolution of CACas9 ΔHbd in the Absence of Butyrate

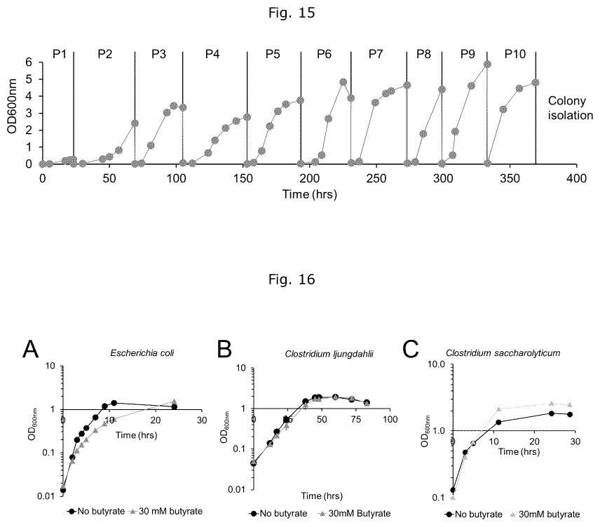

The cell growth of CACas9 Δhbd is significantly slower in the absence of butyrate in the culture medium. The growth phenotype was successfully improved by adaptive laboratory evolution (ALE) by passing the cell cultures to fresh medium over rounds (P1 to P10) in the absence of butyrate (FIG. 15). The faster growing cells derived from CACas9 Δhbd showed normal sporulation and solventogenesis, indicating that pSOL1 plasmid was maintained during the process of ALE. Still butyrate addition further enhanced the growth of the adapted strain.

### Example 15. Effect of Butyrate on Cell Growth of Other Clostridium Strains and Escherichia coli

The positive effects of butyrate on microbial cell growth were never reported before. We further investigated the novel finding by characterizing cell growth of other microbes such as E. coli and other Clostridium strains (FIG. 16). 30 mM of butyrate reduced cell growth rates of E. coli by up to 20%, indicating a toxic effect (FIG. 16A). However, the cell growth rates of C. ljungdahlii were not significantly affected by butyrate (FIG. 16B). Interestingly, the cell growth of C. saccharolyticum was improved by the addition of butyrate (FIG. 16C), suggesting that the addition of butyrate to medium could selectively affect microbial cell growth. Therefore, the positive effect of butyrate on robust cell growth of a 4C pathway deficient microbe is not predictable, and thus an important invention for the microbes where we apply the addition of butyrate.

### Example 16. Addition of Exogenous Gaseous Molecules CO₂ and H₂ or H₂ Alone Improves Carbon Recovery and is Essential for Achieving Supra-Theoretical and Supra-Physiological Metabolite Yields

To establish baseline gas utilization of the coculture between Cac strain CACas9 Δhbd p95ace02a and Clj, we performed an experiment comparing four conditions: Cac monoculture with nitrogen headspace, Cac-Clj coculture with nitrogen headspace, Cac-Clj coculture with 20% nitrogen and 80% hydrogen headspace, and Cac-Clj coculture 20% carbon dioxide and 80% hydrogen headspace. We show that, as hypothesized, coculture with Clj improves carbon utilization. Furthermore, addition of exogenous hydrogen and carbon dioxide further improves carbon utilization relative to the baseline coculture. These experiments were performed in gas tight serum bottles, enabling measurement of the headspace composition via gas chromatography as well as the liquid phase via high performance liquid chromatography.

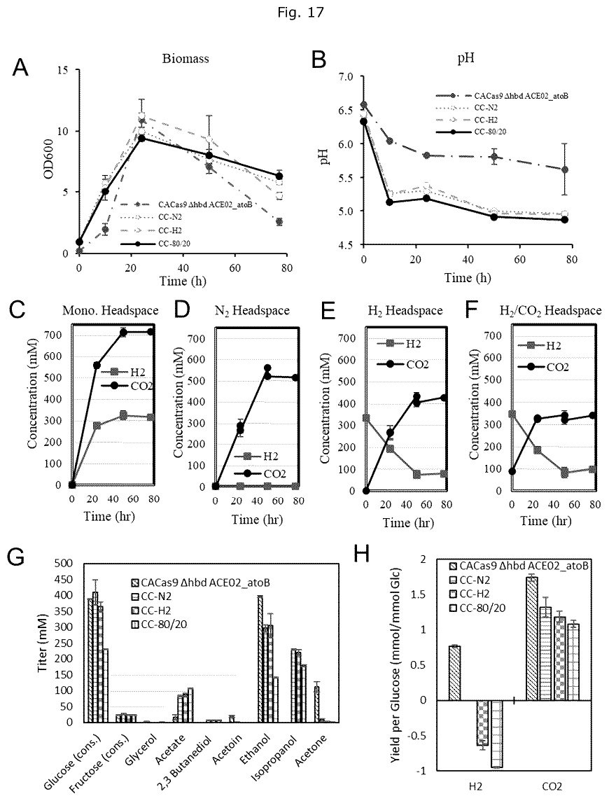

Regarding the overall health and growth of the cells, measurement of biomass via optical density readings showed that the cells grew well to an OD₆₀₀ of between 9 and 12, peaking at around 24 hours, before slowly decreasing throughout the rest of the fermentation (FIG. 17A), as is typical for our Cac monocultures and Cac-Clj cocultures. The pH of the Cac monoculture controls dropped below 6 and then stabilized around approximately 5.8 for most of the fermentation. The cocultures dropped quickly to around 5.2, due likely to the acetic acid produced by Clj, and remained between 5.4 and 4.9 for the rest of the fermentation (FIG. 17B). The pressure of the Cac monoculture rose by nearly 50 psig within 24 hr, requiring the release of most of the gas pressure to avoid rupturing the serum bottle. It accumulated approximately 10 psig more pressure through the course of the fermentation. Coculture with N₂ resulted in the accumulation of approximately 30 psig compared to the start, much less than the monoculture, likely due to Clj's fixation of some of the excess gases. Coculture conditions that started with an 80% H₂ headspace accumulated approximately 12 psig or less, because the additional H₂ provided in the headspace enabled additional CO₂ fixation by Clj. This is demonstrated by the headspace gas profiles and overall gas titers which show zero accumulation of H₂ in the N₂ headspace coculture (because H₂ produced by Cac is immediately consumed by Clj) and net H₂ consumption by the cocultures with 80% H₂ charged to the headspace at the beginning of fermentation (FIGS. 17C-F). The data of FIG. 17E showed that exogenous H₂ alone in the headspace leads to its robust use by the co-culture system leading to better co-culture performance to achieve carbon neutral performance, whereby all the carbon atoms of the carbohydrate are recovered in co-culture metabolites other than CO₂. Thus, H₂ alone is critical for achieving carbon-neutral co-culture performance.

These data of FIG. 17 show that the combination of exogenous CO₂ and H₂ lead to their utilization of the mixotrophic co-culture and are critical for achieving carbon-negative co-culture performance, whereby all the carbon atoms of the carbohydrate plus additional carbons from the exogenously added CO₂ are recovered in co-culture metabolites other than CO₂.

Overall soluble metabolite titers showed that all cocultures produced between 170 and 240 mM of isopropanol. The major product in the monoculture and the N₂ and N₂: H₂ headspace cocultures was ethanol, with titers between 300 and 400 mM. The CO₂: H₂ headspace coculture made approximately 140 mM ethanol, but the major product was isopropanol (FIG. 17G).

When all metabolites are normalized by glucose consumption, the coculture charged with CO₂ and H₂ at the beginning of the fermentation clearly showed the highest yields for isopropanol and acetate per glucose, as well as the lowest ethanol yield. It also had the highest fixation of exogenous H₂ per glucose, and the lowest production of CO₂ per glucose (FIG. 17H).

The same principle of exogenously supplying small molecules to the coculture is applicable to WLP intermediate molecules. Particularly, small amounts and concentrations of CO and formate are important intermediate metabolites from the WLP that can significantly support the CO₂ fixation from the coculture. Large concentrations of CO (of more than 10% are detrimental to Cac and cannot be used (Kim, Bellows et al. 1984)). Moreover, small amount of sugar addition (e.g., <5 mM) that Clj can catabolize will supply required energy (i.e., ATP) to facilitate the WLP and CO₂ fixation.

### Example 17. Improving CO₂ Fixation of Clj Through Fructose Co-Feeding

Acetogens utilizing the Wood-Ljungdahl Pathway (WLP) cannot grow to high cell densities with CO₂ as the sole carbon source (and H₂ as an electron source) due to energetic and regulatory limitations. To support cell growth and biomass accumulation alongside with CO₂, acetogens can be co-fed substrates from which acetogens can derive more energy for growth, notably CO (from which they can extract more biosynthetic energy than the seemingly energetically equivalent CO₂+H₂) or a sugar like fructose (since here, Clj cannot use glucose). However, when provided with an abundance of some sugar substrates, here fructose, carbon catabolite repression (CCR) inhibits CO₂ utilization. We observed this CCR phenomenon when adding a large amount of fructose to Cac-Clj cocultures.

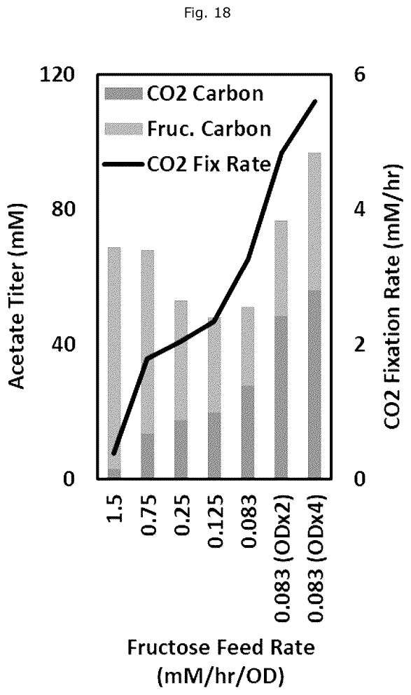

To solve this problem, we investigated a low fructose feeding strategy for cocultures at a rate to support the estimated Clj biomass maintenance, but not enough to activate CCR and inhibit CO₂ fixation. Using constant low feed rates, as opposed to intermittent batch additions, fructose is consumed immediately and does not accumulate in the medium, thus minimizing carbon catabolite repression and maximizing CO₂ utilization. We have conducted preliminary tests of this strategy with Clj monocultures in serum bottles pressurized with CO₂ and H₂ and fed small amounts of fructose through a syringe pump. These results show that CO₂ fixation by Clj improves when fructose availability decreases, and that the percent incorporation of exogenous CO₂ into acetate can be tuned from 4% to 63% simply by varying the specific fructose feed rate (FIG. 18). Critically, these results show that the specific rate of CO₂ fixation remains similar even as cell inoculum density is increased, suggesting that overall CO₂ fixation can be scaled by increasing cell density. Finally, even at our lowest specific feed rate, biomass still increased by 30-84% over the course of the experiment. This suggests that, so far, we have fed fructose in excess of what the cells require to maintain their cell mass. We hypothesize that even lower fructose feed rates will substantially decrease CCR and increase CO₂ utilization once we achieve a high cell density in the bioreactor.

### Example 18. Improving Robustness of Coculture by Adding Sugars to Support the Energy Metabolism of Clj

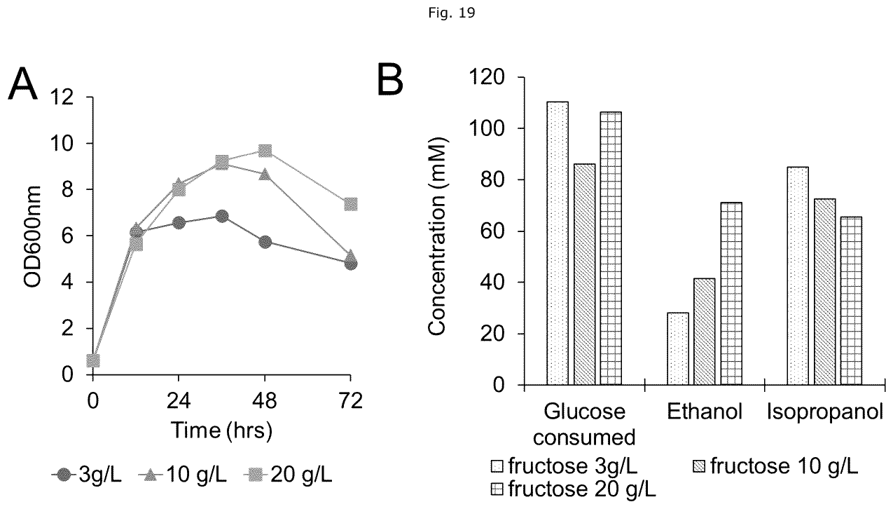

Because fructose is specifically consumed by Clj during the coculture, we tested effects of initial fructose concentrations to the coculture performance (FIG. 19). Interestingly, a better cell mass production was obtained (FIG. 19A), and the isopropanol to ethanol ratio was significantly affected by the fructose concentration (FIG. 19B). The results show that the metabolic shift from coculture is due to the energetic state of Clj. Based on these results, the co-feeding strategy presented above also affects robustness of coculture by supplementing metabolic energy (ATP or equivalent) to Clj, enabling a stable coculture performance without compromised glucose fermentation capability by CACas9 Δhbd.

### Example 19. Development of a High-Throughput Clostridial Interspecies Protein Exchange Assay

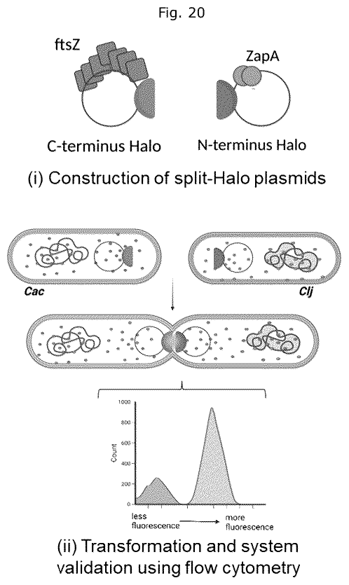

In the syntrophic coculture between Cac and Clj, we have previously observed that the metabolic outcome is simply not a sum of the individual metabolisms of Cac and Clj. These unique metabolic capabilities are due to physical-contact-driven cellular material exchanges between the two microorganisms in coculture. From this interaction, Cac and Clj are able to directly exchange DNA, RNA, proteins, metabolites, and electrons. However, it is not known what could prompt these interactions between Cac and Clj, although a variety of possibilities exist (motility and chemotaxis, quorum sensing, electron transfer). Mechanistic understanding is just emerging (Charubin, Gregory et al. 2021), and Example 42 below demonstrates the importance of expression of a large number of genes. In order to further understand the mechanism behind interspecies interactions, it is first necessary to understand the frequencies at which these events occur amongst the co culture population. To address the challenge, we developed a high throughput clostridial interspecies protein exchange assay using a split fluorescence HaloTag protein compatible with Clostridium species. The split-HaloTag protein covalently bonds to a fluorogenic ligand of choice only when the two divided parts get reconstituted through assembly of the protein partners. For this, protein interaction partners from the Cac divisome (FtsZ and ZapA) are individually fused to each half of the HaloTag protein and transformed separately into either Cac or Clj. During the coculture of Cac and Clj, protein exchange events leading to reconstitution of the HaloTag can be measured using flow cytometry and addition of a fluorogenic ligand (FIG. 20). Time-points at every 6-8 hours are expected to capture cellular exchange events between the two Clostridium species, although the protocol can be adjusted to capture more events for investigation of the interaction mechanism. This high-throughput assay is useful for following applications:
Quantifying the frequency of intercellular interactions between Cac engineered strains and Clj
Determining if Cac is capable of protein exchange with other Clostridium species.
Determining if Clj is capable of protein exchange with E. coli or Clostridium species.
Assessing the impact of motility by either Cac or Clj on frequency of intercellular interactions.
Assessing the impact of quorum sensing or cell signaling on the frequency of Cac/Clj interactions.

### Example 20. High Cell Density Fermentation with Sugar Co-Feeding

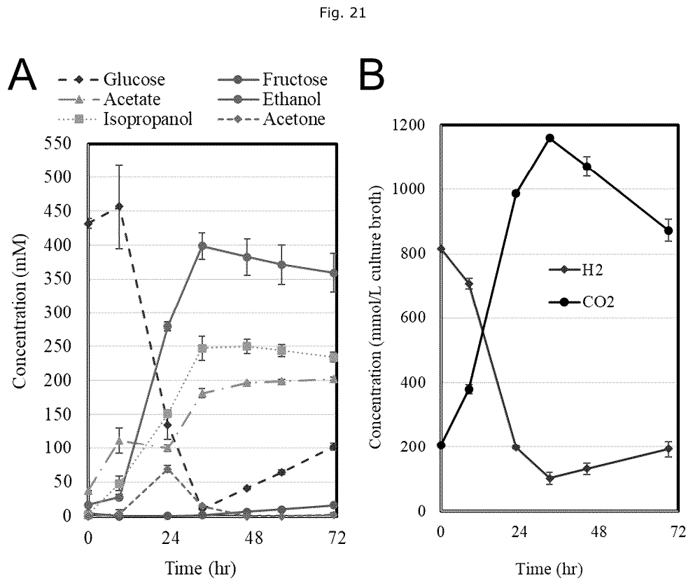

Typically, we start our cocultures around an optical density of 1 with a 10:1 ratio of Clj to Cac. Since interaction between the two species seems to favor improved IPA selectivity, we investigated whether higher density inoculum can increase interspecies proximity from the beginning of coculture. We hypothesized that this approach would increase both IPA selectivity and productivity. First, we conducted experiments in serum bottles in which OD₆₀₀ of Cac and Clj at 1 and 4, respectively. We also connected these bottles with a syringe pump so that they could receive a continuous low fructose feed, to support Clj, and a continuous moderate glucose feed, to support Cac. These cultures accumulated roughly twice the typical amount of biomass (OD₆₀₀ of 20) and demonstrated rapid glucose utilization, H₂ utilization, and IPA production (FIG. 21A). They also produced our highest observed IPA titer thus far of 250 mM, fixed 1.4 mM exogenous H₂ per mM glucose utilized (FIG. 21B), and recovered 78% of the carbon from glucose.

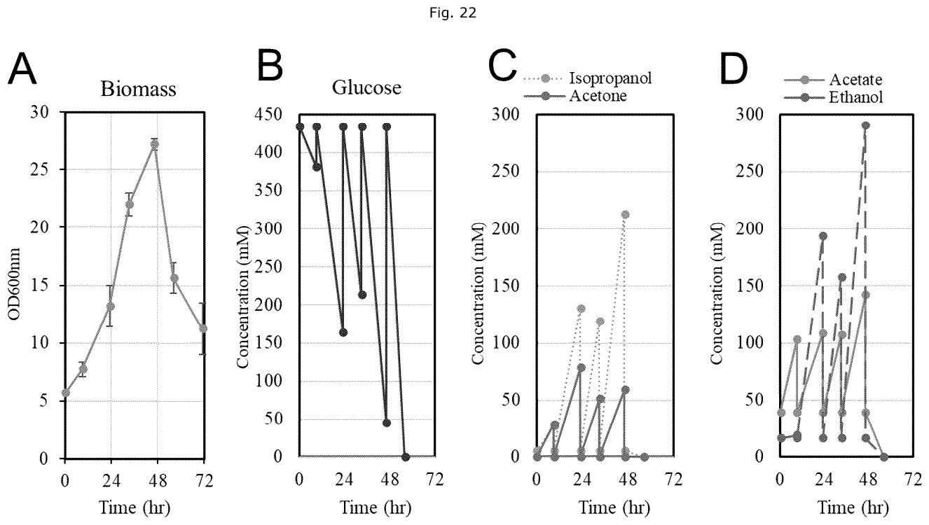

Next, we conducted a repeated batch fermentation with high cell density in 15 mL stationary centrifuge tubes in an anaerobic chamber. We started them at the same 5× starting cell density as the serum bottle experiment, but instead of feeding additional glucose and fructose via syringe pump, we spun down the cells and resuspended them in fresh media every 12 hours. The maximum OD₆₀₀ of the cultures was approximately 27 (FIG. 22A). Of two replicates, one of these cultures achieved glucose utilization rate as high as 22 mM/hr (FIG. 22B) and produced our highest demonstrated IPA productivity of 11.4 mM/hr, with an additional 5.1 mM/hr of acetone (FIG. 22C). As the two byproducts, acetate and ethanol were produced at rates of 7.9 mM/hr and 22.3 mM/hr, respectively (FIG. 22D). The second replicate demonstrated similar biomass accumulation and glucose utilization, but it made proportionally more ethanol, as high as 44 mM/hr. The results show that cell retention significantly improves glucose consumption and IPA productivity.

### Example 21. Carbon Negative Mixotrophic Fermentation

The coculture of CACas9 Δhbd/p95ace02atoB (atoB) and Clj/p100ptaHalo demonstrates carbon recovery of 84% (Cmol metabolites/Cmol glucose) (Table 3). Given the fact that a typical Cac culture uses 3-5% of glucose carbon for biomass, the mixotrophic coculture achieves ˜90% of the sugar-carbon recovery. A higher Clj starting cell densities will substantially improve the carbon recovery of the system, enabling carbon negative fermentation. With precise control and optimization of pH, fructose feed rate, and cell density (via varying the cell recycle rates), we achieve complete glucose utilization and 3-6 additional moles CO₂ fixed per mole glucose utilized.

TABLE 3
Carbon recovery by the coculture of CACas9
Δhbd/p95ace02atoB(atoB) and Clj/p100ptaHalo.

| Metrics | Values |
|---|---|
| C_{substrate} | 1785 |
| C_{product} | 1495 |
| Cₚᵣₒ/C_{sub} | 0.84 |
| U_{substrate} | 830919 |
| U_{product} | 804855 |
| Uₚᵣₒ/U_{sub} | 0.97 |

### Example 22. Stoichiometric Analysis of Accomplishable Product Yields Based on the CO₂ Fixation and Electron Redistribution by the Coculture

In Table 4, we compare current theoretical production capabilities in terms of CO₂ loss and yields, for IPA and, to provide a sense of generalization of the concept, for butanol (BuOH), as well. Table 5 compares the best currently possible biological formation rates and U_{prod}/U_{Glu} values with those proposed.

TABLE 4
CO₂ losses & Max product yields [(Cmol
product)/(Cmol glucose used)]

| | | | | Yield. This invention |
|---|---|---|---|---|
| | CO₂ loss | | Yield: This | with 3 (of 6) mol CO₂ |
| Metab- | per mol | Yield. | invention | fixed per mol |
| olite | Glu: Now* vs | Now* | with H₂ only | Glu (+H₂) |
| IPA | 3 vs. 0 | 0.5 | 1 | 1.5 (2) |
| BuOH | 2 vs. 0 | 0.67 | 1 | 1.5 (2) |

*Monoculture of solventogen (Lee et al., Appl Environ Microbiol 78, 1416-1423 (2012)).
# syntrophic Cac/Clj coculture.

TABLE 5
Rates of product formation (kJ/L/h)
& U_{prod}/U_{Glu} (kJ product/kJ glucose)

| | | U_{prod}/ | Rate | U_{prod}/U_{Glu} |
|---|---|---|---|---|
| | Rate | U_{Glu} | (This | (This |
| Metabolite | Now* | Now* | invention#) | invention#) |
| IPA | 28.5 | 0.72 | 49 (49) | 2.07 (2.76) |
| IPA + BuOH + EtOH | 32.3 | 0.77 | 57 (57) | 2.09 (2.78) |

*Monoculture. Data of (Lee et al., Appl Environ Microbiol 78, 1416-1423 (2012)), reporting the best IPA rates (produced with BuOH & EtOH). Equation: Gluc + H₂O → 1 IPA + 3H₂ + 3CO₂, based on biochemistry & product rates.
#syntrophic Cac/Clj coculture with 3 (or 6; numbers in parenthesis) mol CO₂ fixed per mol Glu (+H₂). 31 mM/h total product formation (Charubin and Papoutsakis 2019) using the following stoichiometric equations. Acetate is recycled for reuse.
(1) 1Gluc + 3CO₂ + 13.99H₂ → 2.28 IPA + 0.57 EtOH + 0.50 Acetate + 8.14H₂O
(2) 1Gluc + 6CO₂ + 22.66H₂ → 3.04 IPA + 0.76 EtOH + 0.67 Acetate + 12.85H₂O
(3) 1Gluc + 3CO₂ + 14.13H₂ → 0.99 BuOH + 1.23 IPA + 0.25 EtOH + 0.43 Acetate + 8.67H₂O
(4) 1Gluc + 6CO₂ + 22.84H₂ → 1.31 BuOH + 1.64 IPA + 0.33 EtOH + 0.58 Acetate + 13.56H₂O

### Example 23. Deletion of a Master Regulator Spo0A to Reduce Ethanol Production

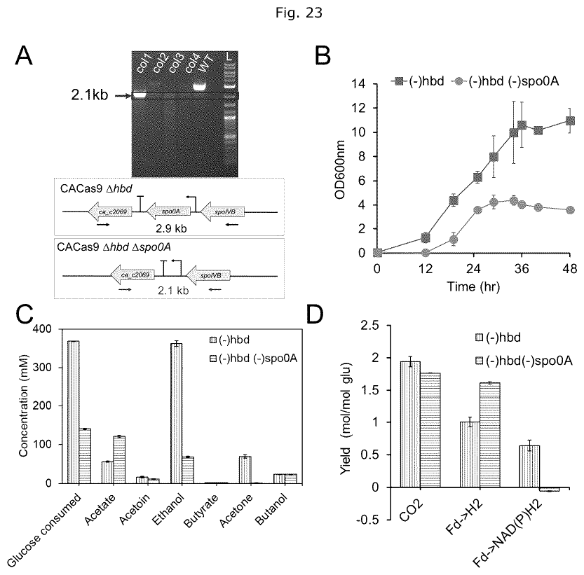

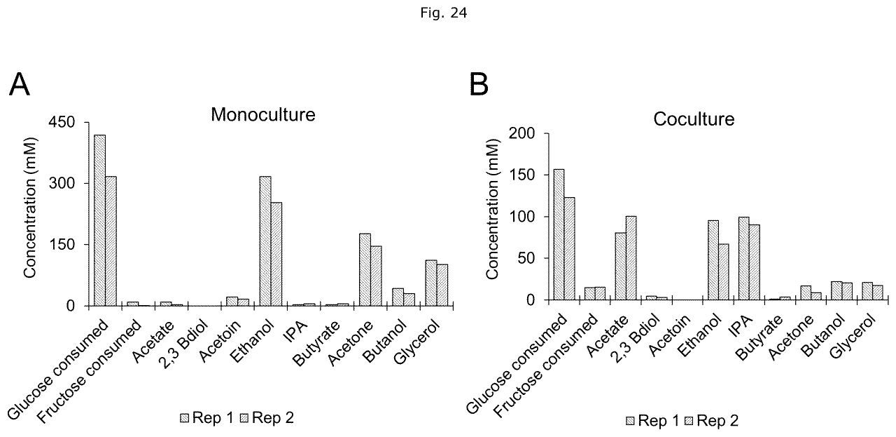

Because ethanol production is facilitated by multiple alcohol DHs, the single gene adhE2 deletion from CACas9Δhbd does not have a significant impact on ethanol production because of existence of several other alcohol DHs. Although the CRISPR/Cas9 tool enables relatively rapid gene deletion, it still takes significant effort and time to execute >3 gene deletions. Therefore, while continuing the alcohol dehydrogenase (DH) deletions, we sought to repress the ethanol production through deleting the gene of the master regulator of sporulation spo0A. Spo0A also governs the expression of many solventogenic genes. Using CRISPR/Cas9, we successfully deleted spo0A gene from the chromosome of CACas9Δhbd (FIG. 23), generating the CACas9ΔhbdAspo0A strain. Its maximum OD₆₀₀ nm was significantly lower compared to the parental CACas9 Δhbd strain (FIG. 23B). As expected (Harris, Welker et al. 2002), CACas9 Δhbd Δspo0A did not produce acetone due to the control of the sol operon genes (that include the genes for acetone production) by Spo0A (FIG. 23C). The CACas9Δhbd Δspo0A strain produced acetate as a primary product up to 116 mM with significantly reduced ethanol production (65 mM). Based on stoichiometric modeling, CACas9ΔhbdAspo0A produced H₂ up to 1.6 mol/mol glucose, corresponding to 60% higher yields compared to CACas9Δhbd (FIG. 23D). Interestingly, the improved H₂ production flux was largely attributed to the net-negative Fd-NAD (P) H reduction. This shows that most of the electrons from Fdred were used for H₂ production instead of ethanol production. The CACas9ΔhbdAspo0A containing a strong acetone production plasmid (p95ace02atoB) produces acetone up to 177 mM from 418 mM of consumed glucose (FIG. 24A). As the byproduct, ethanol and glycerol are produced up to 316 mM and 111 mM, respectively. Coculture of the CACas9ΔhbdAspo0A containing p95ace02atoB with Clj containing p100ptaHalo plasmid produces isopropanol 90-99 mM from 123-156 mM of consumed glucose (FIG. 24B), corresponding to the yields of 0.32-0.37 (Cmol/Cmol). The total metabolite carbon yields (ethanol+acetate+isopropanol) are 0.69-0.82 (Cmol/Cmol).

### Example 24. Deletion of the Rex Gene to Enable Robust Coculture

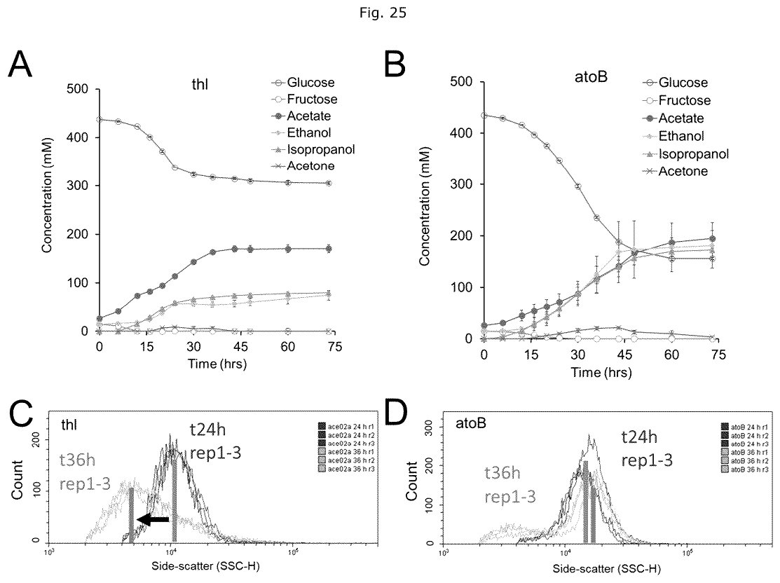

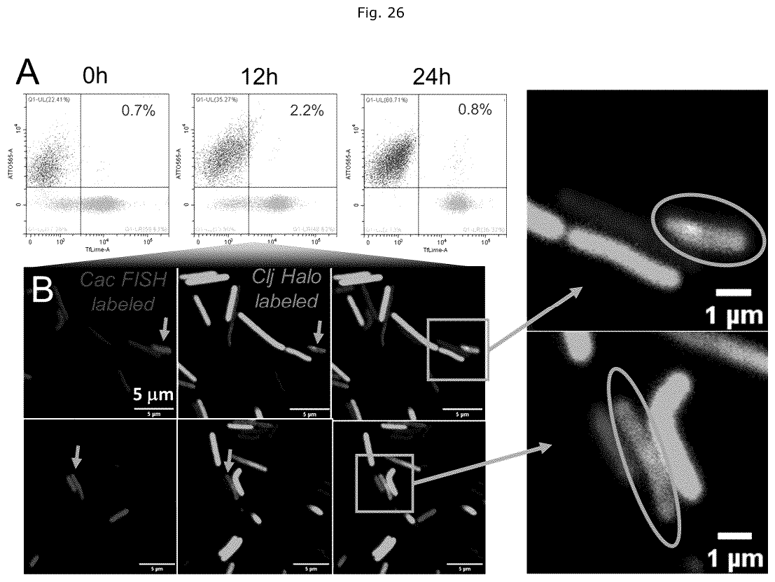

Based on the growth cessation of the CACas9Δhbd/p95ace02a strain when cocultured with Clj, although there was no growth defect when grown in monoculture. The problem was largely resolved by employing (in the acetone production plasmid) a redox-insensitive thiolase (AtoB, atoB) from Escherichia coli instead of the native thiolase, which is known to be regulated by the redox state of the cell. This shows that the phenomenon is related to the native redox sensitive regulation of Cac. We investigated this novel phenomenon through detailed analysis using fermentation kinetics, flow cytometry and microscopy to better understand the reasons (FIGS. 25 and 26). We compared the coculture kinetics using the two strains: CACas9 Δhbd/p95ace02a(thl) (FIG. 25A) and CACas9 Δhbd/p95ace02atoB(atoB) (FIG. 25B). The CACas9 Δhbd/p95ace02a(thl) coculture showed sudden inhibition of glucose consumption around 24 hrs during the fermentation (FIG. 25A). This was in contrast to the CACas9 Δhbd/p95ace02atoB (atoB) coculture (FIG. 25B) which displayed sustained glucose utilization. Flow cytometry data showed that CACas9A hbd/p95ace02a (thl) cells abruptly turned unhealthy after 24 hours compared to the atoB thiolase containing strain (FIG. 25C,D), clearly indicating that CACas9 Δhbd/p95ace02a (thl) cells were detrimentally affected by Clj. We have demonstrated that this cell inhibition did not occur without cell-to-cell physical contact. The cell-to-cell physical contact reduced ethanol but increased acetone production by Cac, suggesting that Cac transferred electron directly to Clj. Flow cytometry of the coculture with RNA-FISH labeled CACas9Δhbd/p95ace02a (thl) and Halotag labeled Clj/p100ptaHalo showed the cell-to-cell material exchange up to 2.2% of the entire population at 12 hrs (FIG. 26A). From fluorescence microscopy, several cells showed double fluorescence signals (FIG. 26B), indicating that the cells exchanged RNA and/or protein.

Taken altogether, Cac directly transfers electrons to Clj via direct physical contact. The direct interspecies (Cac to Clj) electron transfer results in an oxidized redox state of Cac cells, and this is expected to activate gene regulations by the canonical redox sensitive Rex regulator. Based on previous studies, multiple glycolytic genes such as gapC and pfor are likely regulated by Rex. Repression of the genes by Rex would critically affect cell growth and glucose catabolism. Therefore, deleting the rex gene resolves the growth cessation issue and improves robustness of the coculture.

### Example 25. Addition of Butyrate to Maintain Robust Cell Physiology of the Cac Δhbd

Since the development of the CACas9Δhbd strain, our lab has independently observed differences in growth when the strain is grown with and without butyrate.

Without butyrate supplementation on the 2×YTG agar plate, CACas9Δhbd cells appear small in size and grow slowly. Inoculating the cells into liquid media without butyrate leads to a severe lag phase of 3-4 days. To further improve the cell health, it is necessary to passage the cells again to adapt them to the media. However, even after this adaptation, the cell's max OD is only about 4-5. Supplementing the CACas9Δhbd cells with 30 mM of sodium butyrate reduces the lag phase of cell growth noticeably and improves the maximum OD to above 14. As a result, the butyrate addition has positive impacts on the growth of Cac lacking the four-carbon pathway.

The butyrate addition supplies butyryl-phosphate and butyryl-CoA which are key precursor metabolites for post-translational modification (PTM) in Cac, leading to observable changes in the onset of clostridial growth patterns. For example, cell physiology of Cac typically goes through changes throughout its cell cycle, including the clostridial form (large, swollen, cigar shaped solvent producers), and large, complex endospore-forming cells. Further, bright-phase microscopy visualizes the changes in growth we observe from the CACas9Δhbd culture with and without butyrate, and further elucidates the effects of PTM on the sporulation program. We streaked out CACas9 (-hbd) cells onto fresh 2×YTG plates containing butyrate at the following concentrations: 0 mM, 30 mM, and 100 mM). After leaving the plates to incubate at 37° C. for 6 days, we harvested a bacterial smear sample from each condition onto glass microscope slides. We then performed an endospore stain using the Schaeffer-Fulton method, in which spores are stained green by the primary malachite green stain steamed into the bacterial sample, and vegetative cells are counter-stained with safranin (light pink). In comparing the differences in cell physiology between the cells without butyrate and the cells supplemented with 30 mM butyrate, there was a stark difference in the number of endospore-forming cells with butyrate addition. This shows that the clostridial sporulation program is accelerated with the addition of butyrate to the cell growth media in cells lacking the four-carbon chemical pathway.

### Example 26. Construction of a Redox Sensitive Fluorescence Protein Expression Platform

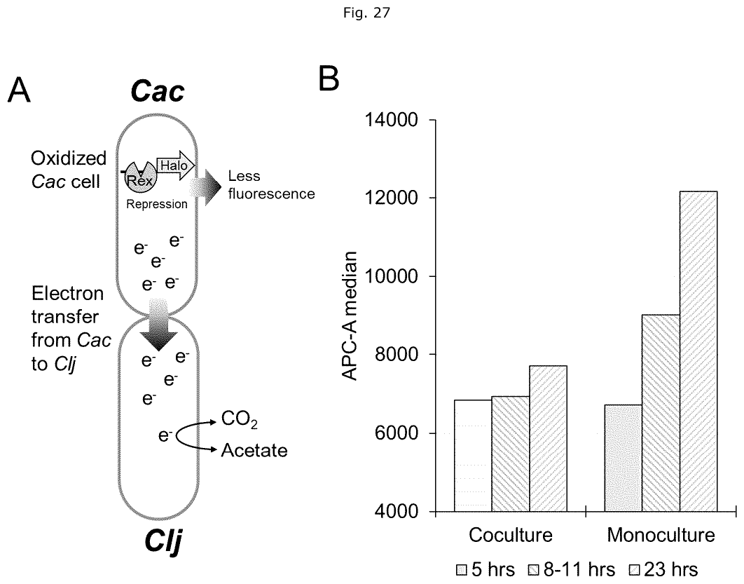

The drastic metabolic shift from coculture of Cac and Clj suggests that Cac cell is relatively oxidized under the coculture setting compared to the monoculture. To further validate the redox change via direct interspecies electron transfer from Cac to Clj, we constructed a redox sensitive fluorescence protein expression system compatible with Cac or other Clostridium species that regulates gene expressions using a redox sensitive regulator Rex protein. The plasmid p100ptarex_Halo consists of a fluorescence HaloTag protein under the control of pta_clj promoter hybrid with the rex binding sequence (5′-aatagtttgttaaatatcaaactaataa-3′). The constructed plasmid functions as a redox sensor by varying expression levels of HaloTag protein (FIG. 27A). Therefore, by monitoring intensity of the fluorescence in the presence of the ligand (e.g., Janelia fluor), redox state of the Cac can be analyzed. This system identifies that coculture significantly oxidizes the Cac cell compared to the monoculture (FIG. 27B).

### Example 27. Identifying Bottlenecks for Selective Acetone Production

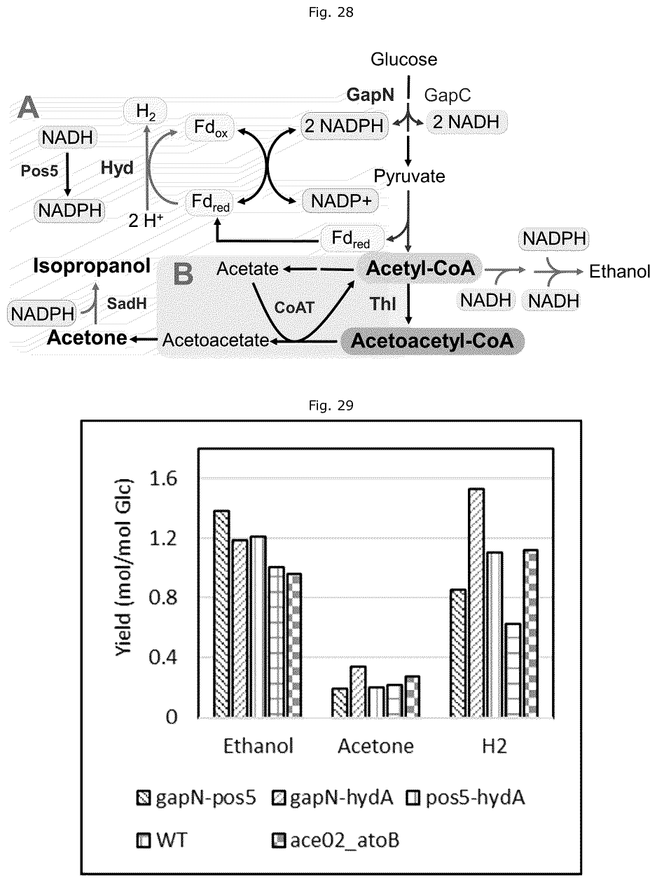

The previously constructed CACas9Δhbd strain (with a deleted 4C pathway that leads to butyrate and butanol formation) produced ethanol as the major product, which competes with acetone production at the acetyl-CoA node (FIG. 28). Overexpression of the acetone production pathway genes from plasmid improved acetone titers up to 120 mM while ethanol was produced at 440 mM, still being the major product. Because acetone production does not involve NAD (P) H consumption, a strong flux towards acetoacetate is necessary to compete with the ethanol production that consumes NAD(P)H for redox balance (FIG. 28). Due to the lack of butyrate production, a strong CoA transferase (CoAT) activity assimilating acetate is critical. Therefore, we aim to employ heterologous CoA transferase (CoAT) and thiolase (Thl) genes originated from microbes without a 4C pathway. As a starting point, we constructed a new acetone production plasmid harboring CoAT and Thl genes from E. coli (atoDA and atoB, respectively). AtoDA has a KM of 53 mM for acetate, which is 20-fold lower compared to the Cac's native CoAT. Characterization of CACas9A hbd/p95ace02atoDAB for acetate assimilation and acetone production is ongoing.

### Example 28. Electron Flux Engineering for Improving Acetone Selectivity

Another reason for the strong ethanol production is the insufficient rate of ferredoxin reduction (e⁻+Fdₒₓ=Fd_{red}) and H₂ production (Fd_{red}=H₂). We found that individual overexpression of Cac's primary hydrogenase (hydA), glyceraldehyde-3-phosphate dehydrogenase (gapN), and a yeast NADH kinase (pos5) gene can improve H₂ production by Cac. hydA overexpression improves H₂ production rate by increasing conversion of reduced ferredoxin to H₂, and gapN and pos5 overexpression improve H₂ production by increasing production of NADPH which can be converted to reduced ferredoxin (and then H₂) by Cac's NADPH-Fd reductase. We thus tested combinatorial expression of these three H₂-production enhancing genes, hypothesizing that they would work together synergistically to direct electrons towards H₂ and away from ethanol (FIG. 28). We constructed and tested three plasmid constructs for overexpression in Cac: gapN-pos5, gapN-hydA, pos5-hydA. All the genes were expressed from monocistronic operons under the control of the two strongest known promoters (Ppta_clj and Psthl). To evaluate the performance of each new strain, we performed a simple monoculture fermentation in serum bottles, measured the soluble metabolites, normalized the metabolite production by the amount of glucose consumed, and compared the results to previously obtained results for the baseline CACas9Δhbd strain as well as the CACas9Δhbd strain transformed with our best acetone plasmid obtained thus far, p95ace02atoB (ace02_atoB) (FIG. 29). These results show that all three new constructs improved H₂ production compared to the plasmid free control (WT). The best strain was determined to be gapN-hydA, which resulted in 2.4- and 1.4-fold higher H₂ yields compared to the WT and ace02_atoB, respectively. Interestingly, gapN-hydA also outperformed ace02_atoB in terms of acetone production, improving acetone yields by 24%. The results proved our inventive hypothesis that manipulating of electron flow is important for controlling acetone production. This strategy is applied to genomic integration since this collection of acetone production pathway genes and the electron flux engineering genes is too large (˜11 kb) to express from a single plasmid.

Another interesting result was that the gapN-pos5 overexpressing strain produced 58% more ethanol than the baseline strain with no plasmid, contrary to our hypothesis. We expected overproduction of NADPH via gapN and pos5 to decrease ethanol production since the adhE1 and adhE2 genes from the pSOL1 megaplasmid cannot utilize the NADPH for ethanol production. The results can be explained by the fact that the three chromosomal NADPH-dependent butanol dehydrogenases (DHs) bdhA, bdhB, and bdhC are capable of taking over ethanol production using NADPH.

Therefore, multiple alcohol DHs including adhE1, adhE2, and bdhABC were deleted to decrease ethanol production through genetic modification. Particularly, BdhA/B/C are responsible for over 95% of NADPH-dependent alcohol dehydrogenase activity in Cac (Yoo, Bestel-Corre et al. 2015) Therefore, we knocked-out both bdhA and bdhB (and also bdhC if necessary), while simultaneously knocking in the synthetic acetone production operons as well as hydA and gapN.

### Example 29. Enhancing Clj Growth and Activity in Coculture to Increase CO₂ Fixation

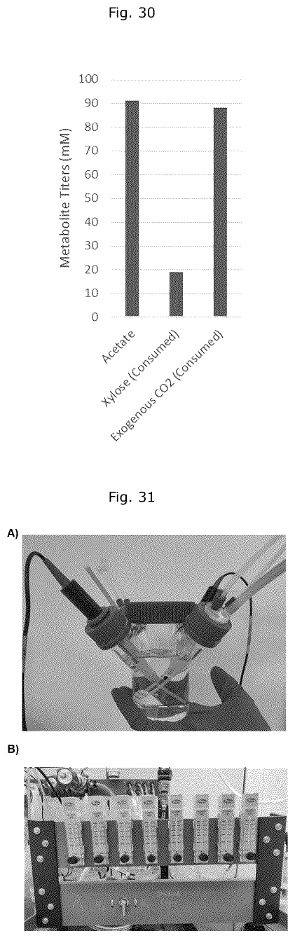

Although our cocultures have consistently demonstrated substantially improved carbon recovery relative to the monoculture, we aimed to achieve carbon negative fermentation or complete utilization of all the CO₂ produced by Cac via glycolysis when using the carbohydrate substrates. This suggests that we need to increase the activity of Clj in our cocultures since Clj is the coculture partner responsible for CO₂ fixation via the Wood Ljungdahl Pathway. Since mixotrophic growth of Clj on some small amount of fructose, CO₂, and H₂ is faster than purely autotrophic growth on CO₂ and H₂, we hypothesized that adding fructose to the cultures would increase the growth and activity of Clj, leading to higher overall CO₂ fixation. However, analysis of our data showed using high concentrations of fructose actually decreased the carbon recovery, despite improved growth and acetate production by Clj. We tested Clj monocultures with different fructose feed rates and showed that too much fructose decreases Clj CO₂ utilization due to carbon catabolite repression (FIG. 18). To get around this issue, we tested a variety of sugars to find one that Clj could utilize for extra energy but that does not attenuate the simultaneous CO₂ utilization due to catabolite repression. We found that xylose can be consumed by Clj at the same time as CO₂. Batch Clj monocultures fed 5 g/L xylose and exogenous CO₂ and H₂ fixed 48.3% of products from exogenous CO₂ with over residual 2 g/L xylose remaining at the end of fermentation (FIG. 30). We also found that, by decreasing the rate of fructose feeding such that it did not accumulate in the medium at a high enough level to trigger carbon catabolite repression, we could substantially boost the CO₂ utilization by Clj monocultures relative to cultures fed with higher rates of fructose or a large batch of fructose at the beginning of the culture. Arginine was also found to increase autotrophic growth rates of Clj without attenuating CO₂ utilization. Since Cac will not utilize xylose or fructose in the presence of high enough levels of glucose, and Clj cannot utilize glucose, we were able to orthogonally support growth of and CO₂ utilization by Clj in cocultures with Cac via xylose addition, arginine addition, or slow fructose feeding, or some combination of all three strategies. Additional chemicals that could substitute for xylose and arginine included pyruvate and TCA cycle intermediates.

### Example 30. Optimized rRNA-FISH for Orthogonal Labeling of Cac Ribosome

Ribosomes are macromolecular machines capable of performing protein synthesis. Ribosomes are constructed from ribosomal RNA scaffolds (rRNA) and ribosomal proteins (RPs). In bacteria, ribosomes are the combination of two-subunits: the 50S and 30S subunits. The 50S subunit contains a 23S rRNA and 5S rRNA, whereas the 30S has a single rRNA: the 16S rRNA.

Ribosomal ribonucleic acid fluorescence in situ hybridization (rRNA-FISH) is a technique for labelling ribosomes through the formation of duplexes between a fluorescently labelled rRNA-FISH probe and a complimentary sequence of RNA in the ribosome. The rRNA-FISH probe is a short DNA fragment with an associated fluorescent molecule which bears homology to the target rRNA sequence. In bacteria, the probe can be designed to bind to the 23S, 16S or 5S rRNA. The probe can bind to conserved sequences which are found in many species of bacteria or species-specific regions, in which case the rRNA-FISH probes will only bind to the ribosome of a specific species of bacteria. We developed this technique for use in our lab to confirm material exchange during cell fusion between microorganisms in a synthetic consortia. First, we designed and synthesized species-specific probe targets for Cac and Clj: ClosAcet and ClosLjun. ClosAcet has the sequence 5′-CCTGACGGAACTGCTTCC-3′. ClosLjun has the sequence 5′-CGCCACTACTTCCTAGTC-3′. During synthesis both are bonded to a fluorescent molecule at the 5′ end. Second, we optimized a pre-existing FISH protocol to be less time consuming without sacrificing labelling efficiency. Because the method is performed in-solution rather than on a slide, which is the traditional implementation of the method, we call this method ‘in-solution RNA-FISH.’ During processing, culture samples were diluted or concentrated to an OD₆₀₀ of 0.5 and washed twice in filter-sterilized ice-cold 1×PBS (Gibco, pH 7.4) via centrifugation (5-10 min, 3220 rcf, 4° C., Centrifuge 5810 R; Eppendorf). Then the pellet was resuspended thoroughly in 5 ml of ice-cold 1×PBS, and fixed by 1:1 dilution in ice-cold absolute ethanol. All samples are stored at −20° C. for up to a month.

For each in-solution-RNA-FISH sample, 300 μl of cells were pelleted via centrifugation 10 min., 10000 rcf, 4° C., Z216 MK; Hermle) in a 1.6 mL microcentrifuge tube. After carefully removing the supernatant, the pellet was dried for 5 to 15 minutes at 46° C. (Isotemp Hybridization Incubator; Fisher Scientific) to remove residual ethanol. The pellet was resuspended in 75 μl of hybridization buffer containing 0.9 M NaCl, 0.02 M Tris-HCl (pH 7.0), 20% (v/v) formamide, 0.01% sodium dodecyl sulfate, and 1 μM probe(s) in aqueous solution and incubated at 46° C. for 5 hours. Immediately following hybridization, the cells are pelleted via centrifugation (8 min., 10000 rcf, Centrifuge 5418; Eppendorf), and the supernatant is discarded into formamide specific waste stream. To mitigate promiscuous probe binding, the pellet is resuspended in 500 μl of pre-warmed washing buffer, incubated for 25 min at 48° C., washed once and resuspended in 1 mL of ice-cold 1×PBS.

This method is used to identify cells participating in interspecies protein exchange in the case that the synthetic consortia contains cells from a species (other than the target species) expressing a fluorescent protein. Following the above protocol, the cells are interrogated via flow cytometry or microscopy or both. Cells containing visual signals from both the rRNA-FISH probe and exogenous fluorescent protein can be considered participating in interspecies protein exchange.

Since the rRNA-FISH probes are species-specific, they may be used to identify “hybrid cells’ which contain transiently or constitutively rRNA sequences from both species. Following the above protocol, the cells may be interrogated via flow cytometry or microscopy or both. Cells containing visual signals from both probes contain both types of rRNA and can be considered hybrid cells.

### Example 31. Improving in-House Mini Bioreactors for Two-Way pH Control and Gas Feeding

To enable the controlled feeding of hazardous gases, we have fully initialized and tested a new small-scale bioreactor system capable of handling H₂, CO₂, and CO in a protected and dedicated hood. It includes all the safety measures for continuous, unmonitored hydrogen fermentation, including its own dedicated hood, a hydrogen meter and alarm, a flashback arrestor, backflow preventers, high-grade tubing, and a precise regulator. It also incorporates two-way pH control for four bioreactors or one-way pH control for eight bioreactors. New, custom-designed glass vessels with multiple sidearms for multiple probes and multiple liquid and gas connection points were commissioned and received (FIG. 31A). These vessels will be used for a gas recycling and retention system that will be able to capture, analyze, and reuse all fermentation gases. Their additional connection ports are used for cell retention inputs and outflow and for automatic sugar and nutrient addition. Additionally, new gas flowmeters have been added for each bioreactor (FIG. 31B). They allow for the precise monitoring and control of the gas feed rate to each reactor, whether that gas is being freshly fed from a tank or recycled. These vessels overcome several deficiencies from past bioreactor test beds, such as lack of ability to tolerate flammable gases, lack of two-way pH control, non-integrated sugar feeding, and overinvolved tubing and cabling that made fast reuse challenging. These reactors are used for many pH tolerance and gas feeding experiments, and we anticipate their continued use both for this clostridial fermentation and for other target clostridial or other microbial fermentations. They are also used to help generate scale-up plans for this fermentation with our industrial partner. This system saves at least 75% of the cost of an Eppendorf DasBox or competing system with minimal loss in essential capabilities.

### Example 32. Cell Resuspensions in pH-Controlled Bioreactors with Continuous Gas Feeding

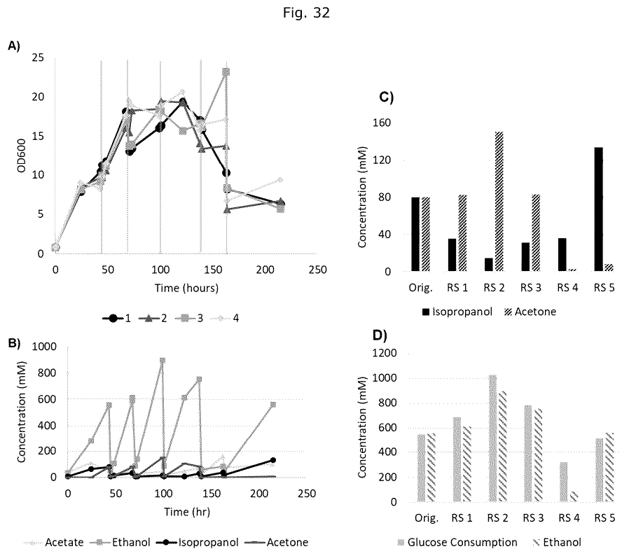

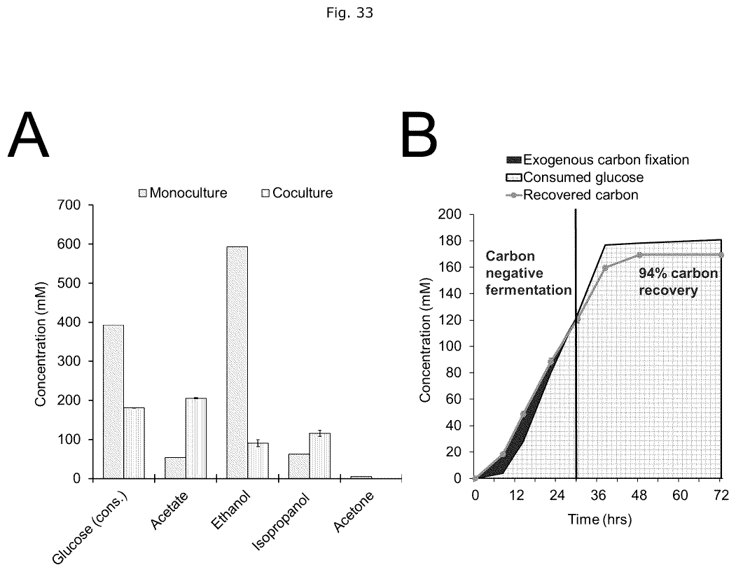

In light of the results seen in repeated batch fermentations where cells were centrifuged and resuspended every twelve hours, we conducted a similar experiment in the pH-controlled bioreactors described in Example 31. Results from this experiment are seen in FIG. 32. Four different bioreactor vessels with the same initial starting inoculum were used, and the OD values over time are seen in FIG. 32A. Maximum ODs of ˜21 are seen. The cells were grown with continuous feeding of ˜6 cc/min 80% H₂/20% CO₂ and continuous feeding of glucose. The cells are resuspended every ˜30 hours, as noted by the vertical gray bars. Cells were aspirated from the bioreactors, which maintained their hydrogen and carbon dioxide headspace, with 60-mL syringes connected to sampling ports until the bioreactors were virtually empty. The capped syringes were taken inside an anaerobic chamber and their contents transferred to sterile 50 mL centrifuge tubes, which were spun for 10 minutes at 5,000 rpm. The cell pellets were then resuspended in ˜30 ml of fresh T-CGM media before being reintroduced to the bioreactors, which had been filled with 150 mL of fresh, degassed T-CGM media. A longer distance between each resuspension was chosen so that the pH control and continuous glucose feeding would lead to the increased stability of the coculture between resuspensions, but more frequent resuspensions could be tested to approximate a higher rate of media recycling. This leads to higher cell mass and increased production of targeted chemicals.

The metabolite concentrations of the most successful of the four reactors are seen over time in FIG. 32B and in detail in FIGS. 32C-D. Glucose consumption rises with each resuspension until 3, which further lowers at resuspension 4. This shows that more frequent resuspensions or dosing of vitamins and minerals are needed. However, ethanol continues to be produced in high quantities (˜600-800+mM), and both cell populations endure. After the coculture recovers after the fourth resuspension, isopropanol levels are the highest in the entire coculture, at ˜130 mM. We will get more isopropanol production with more genetic engineering to shift carbon from ethanol to isopropanol. More frequent dosing of Clj, adjustments to the starting ratio of the coculture, or genetic engineering of Cac to incorporate isopropanol production or internal acetone to isopropanol production suppress the acetone buildup seen especially in resuspensions 1-3. Additionally, fully-integrated cell recycling, without the labor or cell stress of centrifugation, further improves isopropanol production. When bioreactor glucose carbon utilization matches that seen in cocultures in bottles or centrifuge tubes, chemical production more than doubles if these glucose consumption levels are held constant.

### Example 33. Overexpressing a Secondary Alcohol Dehydrogenase (sadH) to Enhance Conversion of Acetone into Isopropanol

With the goal of reducing ethanol production from the coculture to bolster IPA selectivity, we constructed a plasmid harboring our best acetone operon along with the NADPH-dependent SADH gene Clostridium beijerinckii (sadH). The resultant plasmid (p95IPA01atoB) was transformed into the CACas9 Δhbd strain, generating CACas9 Δhbd/p95IPA01atoB. Under the monoculture condition, CACas9 Δhbd/p95IPA01atoB consumed all of glucose provided (80 g/L) within 48 hours (FIG. 33A). The cultures readily converted acetone to isopropanol through the heterologous expression of the sadH, leaving only trace amounts of acetone in the medium (<5 mM). However, the strain still produced ethanol as a major product (>600 mM) with only 60 mM of IPA, resulting in IPA/EtOH molar ratio of less than 0.1.

Interestingly, when CACas9 Δhbd/p95IPA01atoB was cocultured with Clj/p100ptaHalo, the IPA/EtOH ratio improved dramatically to higher than 1.3 (115 mM IPA/90 mM ethanol). With this improved coculture, the IPA yield reached up to 0.64 (mol/mol glucose), and the sugar-carbon recovery reached above 100% for the first 22 hours of the culture (FIG. 33B). The overall carbon recovery for the 72 hours coculture reached 94% from the three biological replicates, an exceptional figure on its own given that, stoichiometrically, for every mol of acetone or IPA from glucose, 3 mol CO₂ must be released. Based on the strong performance, carbon recovery and isopropanol yields will be further improved in conjunction with high-cell density coculture that enables rapid glucose conversion into IPA as well as higher carbon fixation rates. Given the fact that a significantly high level of acetate accumulates, further engineering efforts for improving acetate assimilation leads to better IPA production with effective CO₂ fixation. We have successfully transformed Clj with the acetone production plasmid (p100ace02a). With improved CoAT and thiolase activities in both Cac and Clj, we obtained enhanced conversion of acetate into IPA.

### Example 34. High-Cell Density Fed-Batch Coculture with Cell Retention Achieves High IPA Productivities, Yields, and Selectivity

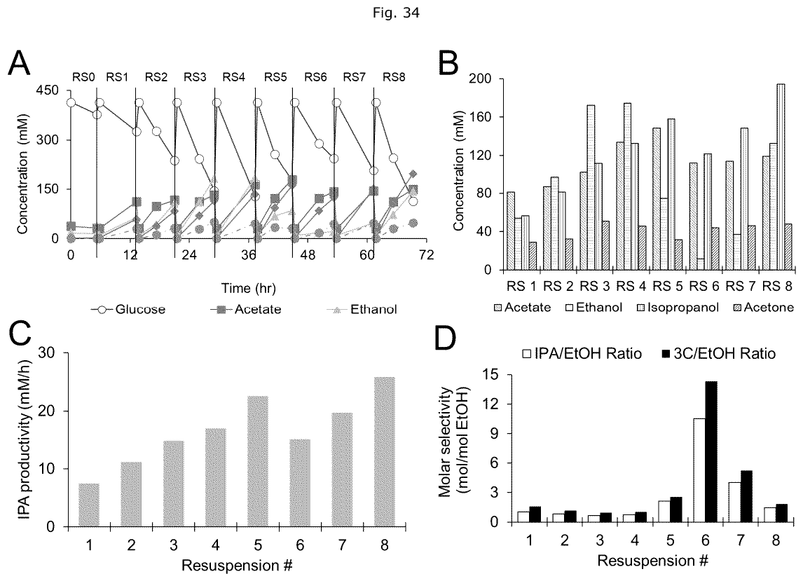

Based on the promising performance by high-cell density coculture from the previous example, we scaled up the experiment to serum bottles with the additional CO₂/H₂ supplementation at the headspace. We performed a repeated-batch fermentation by resuspending the coculture cell biomass into the fresh medium with H₂/CO₂ headspace with additional Clj inoculation every eight hours. The additional Clj helped conversion of the acetone into isopropanol with improved CO₂ fixation. The maximum optical density (OD₆₀₀) reached 45 and maintained over the course of fermentation. Headspace pressures increased to 20-30 psi during some fed-batch cycles, which indicates that CO₂ was produced that Clj was not able to assimilate. With this experimental design, after each cycle, the headspace was flushed with H₂ and CO₂ in an 80/20 ratio and the pressure was reset to 15 psi so that adequate H₂ would be available. The pH was maintained between 5.0 and 5.5 without any intervention. FIG. 34 shows the glucose consumption, metabolite titers, IPA productivities, and IPA selectivity from the high-cell density coculture experiment.

The IPA titers ranging from 120 to 200 mM were achieved after the third cycle (RS3), with productivities between 14.9 and 25.9 mM/h (FIG. 34A, B, C). The productivity of 25.9 mM translates into 1.46 g/h/hr, which is at the 90% level of the proposed productivity goal of 1.61 g/L/hr. We observed acetone accumulation up to 50 mM which will be additionally converted to IPA by employing the better performing Cac and Clj strains. Surprisingly, the IPA selectivity of 10.5 was achieved from the RS6 batch (FIG. 34 D). Considering the accumulated acetone, selectivity of the 3C product (IPA+acetone) over ethanol reached 14.3 (mol/mol). Because ethanol is the major electron sink of Cac, the high IPA production from the coculture further highlights the synergism of the coculture system regarding the electron and energetic coupling between Cac and Clj. The maximum IPA and 3C product yields were 0.72 (mol/mol glucose) and 0.98 (mol/mol glucose), respectively. The yields of 0.98 is 67% of the maximum theoretical IPA yields (1.5 mol/mol glucose) with 3 mols of CO₂ fixation per mol of glucose. The high product yields represent effective redistribution of electron and CO₂ fixation into acetone and IPA production by the coculture of Cac and Clj. Because the outstanding performance seems to be related to the high cell density and Cac-Clj cell ratio, we are pursuing maintenance the performance of RS6 with measurement of detailed coculture dynamics. In addition, further engineered Cac and Clj strains with better IPA production and acetate assimilation will be applied to the established coculture scheme. The carbon recoveries were maintained at 73% over the course of coculture. A better carbon recovery is achieved by combining the sugar co-feeding strategy as presented below. Overall, we experimentally demonstrated that high IPA titers, productivity, and selectivity can be achieved via high-cell density coculture in conjunction with cell retention.

### Example 35. Nitrate Addition Shifts Coculture Product Profile from Isopropanol to Ethanol and Suggests Possibility of Nitrogen Based Syntrophy

Previously, we discovered that feeding nitrate to Clj monocultures resulted in effective pH control and, in some cases, an increase in pH, despite the production of large quantities of acetic acid that would normally cause culture pH to drop sharply. This is likely due to the fact that Clj can reduce nitrate to ammonia, a weak base. We hypothesized that adding nitrate would similarly create an effective pH buffering effect in Cac-Clj cocultures.

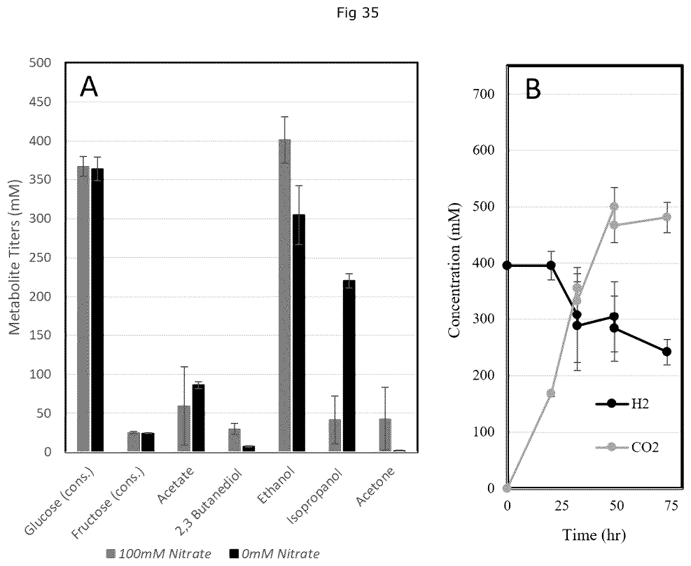

We tested this hypothesis, adding 100 mM of sodium nitrate to the media of a Cac-Clj coculture. As hypothesized, this strategy maintained the coculture pH above 5.75, whereas the pH of the control without nitrate dropped below 5. Interestingly, the addition of nitrate also strongly influenced the metabolite profile, strongly increasing ethanol production and decreasing isopropanol production relative to the control without nitrate. We hypothesize that this is due to the nature of the nitrate to ammonia conversion. In a normal coculture Clj uses electrons, either from hydrogen in the headspace or from Cac via direct electron transfer to Clj, to reduce acetone produced by Cac to isopropanol (IPA). However, in the presence of so much nitrate, Clj instead used its excess electrons to convert nitrate to ammonia, a conversion which requires four reducing equivalents (a “reduction equivalent” refers to chemical species (electron carriers), thus which transfer the equivalent of one electron in redox reactions; thus four reducing equivalents is four electrons). Indeed, not only did we observe substantially less IPA, we saw also accumulation of acetone equal to isopropanol (˜40 mM) in the nitrate control, whereas we saw less than 3 mM of acetone and 220 mM of isopropanol in the no nitrate control (FIG. 35A). Headspace analysis via gas chromatography demonstrated utilization of exogenous H₂ (FIG. 35B), showing that Clj was alive and active in these cultures despite the accumulation of acetone. This is a very interesting result that demonstrates the possibility of tuning the coculture product profile and electron flow via simply using nitrate instead of ammonia, the typical nitrogen source we use in our growth medium. As seen here, this could be used to shift the coculture towards the production of ethanol, as well as perhaps other products with additional genetic engineering of either or both Cac and Clj. Moreover, since Cac does not possess the genes to utilize nitrate and can only use ammonia (based on bioinformatic searches), use of nitrate could be a simple but powerful way to control the coculture population ratio. In a typical coculture Clj relies on Cac for carbon and electrons, but Cac does not require Clj for growth. However, if the two species are cocultured in media containing only nitrate, instead of ammonia, Cac, normally the faster growing of the two species, would be growth limited by Clj's conversion of nitrate to ammonia. This would lead to much greater operational stability and control of the coculture in an industrial setting, as well as strengthen and enhance the metabolic coupling, direct electron exchange, and cell fusion phenotype we have already observed in this coculture system.

### Example 36. Improving Clj Activity Leads to Exogenous CO₂ Fixation and 112% Carbon Recovery in Cac-Clj Coculture

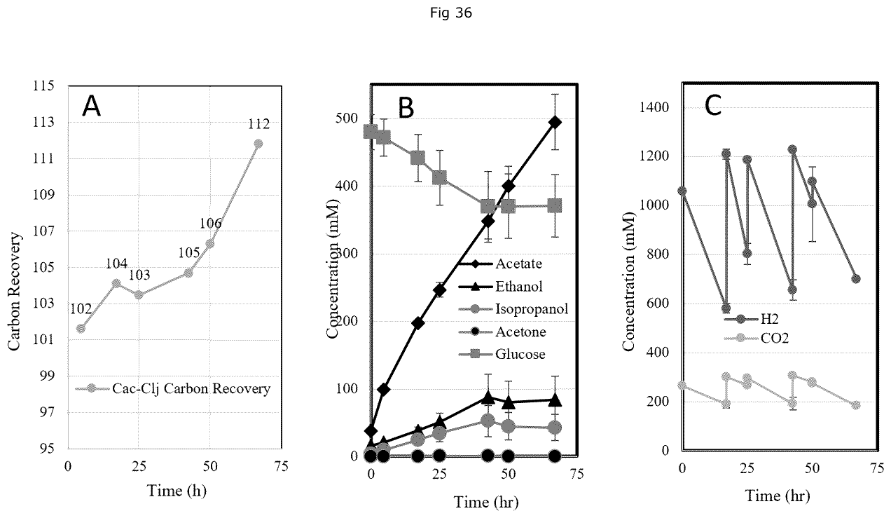

As discussed in the examples above, we demonstrated that the presence of fructose in the culture media inhibits, via carbon catabolite repression, Clj's ability to utilize CO₂. We also showed results to the effect that continuous feeding of low rates of fructose throughout the coculture allows Clj to obtain enough energy to maintain its biomass whilst still fixing large amount of CO₂. This quarter we further optimized this approach, demonstrating 112% carbon recovery in a carbon negative coculture (FIG. 36A) between Cac and Clj.

We determined that a feed rate of 0.060 mM fructose per hour per OD of Clj was sufficient to maintain active Clj cell mass while minimizing carbon catabolite repression and maximizing CO₂ fixation. We chose to start a coculture with a Clj starting OD₆₀₀ of 4 and a starting OD₆₀₀ of 1 of Cac (4:1 ratio of Clj to Cac). We used our normal Clj GTHalo strain and CACas9 4C-expressing our best p95ace02atoB plasmid grown under a headspace flushed with 80% H₂ and 20% CO₂ at the beginning of the experiment and at every timepoint.

These strains and starting condition combined with a fructose feeding rate of 2.4 mM fructose per hour (the rate described above scaled with the starting Clj biomass) led to a coculture that maintained carbon negative conditions throughout the fermentation and finished with a 112% carbon yield relative to sugars consumed (FIG. 36A), showing that extra CO₂ was incorporated from the headspace in addition to all CO₂ produced by Cac. These cocultures accumulated by far the largest amount of acetate we've observed, over 500 mM, suggesting that CO₂ fixation by Clj was extremely active (FIG. 36B). However, glucose consumption was only 109 mM, leading to production of 87 mM of ethanol and 52 mM of isopropanol, suggesting that Cac performance was compromised. Headspace analysis via gas chromatography confirmed that there was net consumption of CO₂ (FIG. 36C).

When we first knocked out the 4-carbon pathway (leading to butyrate and butanol formation) from Cac, we observed that Clj appeared to inhibit Cac's growth in coculture. We hypothesized that this phenomenon is due to redox stress caused by direct electron transfer from Cac to Clj facilitated by cell-to-cell contact. We ameliorated this issue by overexpression of the redox-insensitive thiolase atoB from E. coli in place of Cac's endogenous thiolase. However, now that we have substantially increased Clj's ability to fix carbon and electrons via supporting a higher active Clj cell fraction with fructose feeding, we appear to be running into this issue again. Fortunately, we have demonstrated from our resuspension experiments that this problem can be resolved by maintaining a higher active fraction of Cac. Repeating this experiment with the same starting amount of Clj but a higher amount of Cac (˜1:1 Clj: Cac ratio) maintained a high enough active fraction of Cac to fully utilize the available glucose in the presence of a high density, highly active Clj cell population thus maintaining carbon negative conditions throughout the fermentation.

### Example 37. Fluorescent Material Exchange Between Cac M5 (Strain without pSOL1 Megaplasmid) and Clj Due to Heterologous Cell Fusion

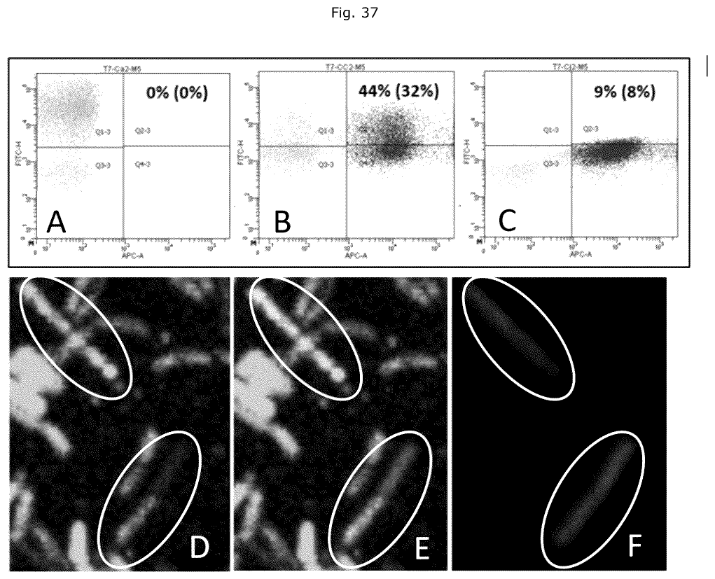

Flow cytometry and confocal microscopy demonstrated the exchange of cytoplasmic material between the asporogenous M5 Cac strain and Clj. For flow cytometry, >40% of labelled cells contained both fluorescent dye labelled RNA from Cac and fluorescent Halotag protein from Clj after 7 hours of coculture (FIG. 37A-C). Confocal microscopy of samples also identified numerous double positive cells. These included cells containing evenly distributed red and green labelling, as well as cells with labelling gradients that appeared to be in the process of receiving material from another cell (FIG. 37D-F) during the process of heterologous cell/membrane fusion.

### Example 38. M5Cas9 ΔHbd/p95Ace02a has Little Synergism with Clj for IPA Production, and does not Fuse with Clj

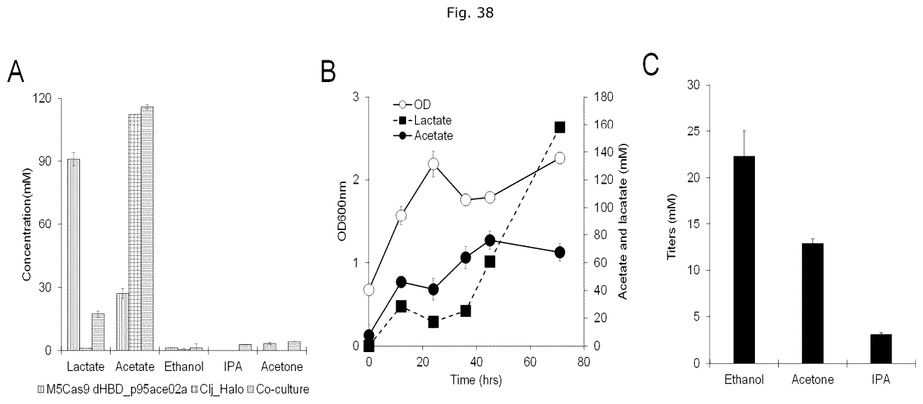

The direct electron transfer from Cac to Clj alters the metabolite profile and improves IPA production. To demonstrate that specific genes are necessary for direct Cac/Clj interactions and cell fusion, and that such genes are likely affected by genetic modifications of the microorganisms, we examined the interactions between M5Cas9 Δhbd/p95ace02a and Clj/p100ptaHalo. M5Cas9 Δhbd/p95ace02a is the asporogenous Cac M5 strain lacking the pSOL1 megaplasmid whereby the Cas9 has been integrated in its genome and used to delete the Hbd coding gene and whereby the acetone formation plasmid p95ace02a has been inserted by electroporation. Clj/p100ptaHalo is Clj microorganism containing the p100ptaHalo plasmid which expresses the Halo-tag fluorescent protein. We measured metabolite profiles of the coculture of M5Cas9 Δhbd/p95ace02a and Clj/p100ptaHalo (FIG. 38). M5Cas9 Δhbd/p95ace02a produced lactate as the major product and produced only 3.3 mM of acetone (FIG. 38A). The coculture produced less lactate compared to the monoculture. However, only 2.7 mM of IPA was produced, which demonstrates that there is no synergism between M5Cas9 Δhbd/p95ace02a and Clj for IPA production. Due to the lactate production, pH rapidly dropped below 4.5. To interrogate the direct electron transfer from M5Cas9 Δhbd/p95ace02a to Clj, cell growth kinetics and the two major products of the coculture (i.e., lactate and acetate) were monitored with pH control (FIG. 38B). Only 25 mM of lactate were produced for the first 36 hours, and the production reached up to 158 mM for the next 36 hours. The final titers of acetone and IPA were 12.9 and 3.1 mM, respectively. These results demonstrate that there is no interspecies material exchange and fusion between M5Cas9 Δhbd/p95ace02a and Clj. Loss of the butyrate and butanol pathways together with the loss of the genes encoded in pSOL1 plasmids suppress the Cac-Clj interspecies interaction. Therefore, one cannot predict the impact of genetic modifications on direct Cac-Clj interspecies interaction, heterologous cell fusion and the exchange of electrons, metabolites and cellular material. This should be contrasted with the results of Example 37. As such the findings of Examples 5, 6, 7, 16, 17, 20, 21, 29, 32, 34 and 36 could not have been predicted based on findings from the cocultures of wild-type (WT, or genetically unmodified) Cac and Clj strains. Genetic modifications change the expression of many unrelated genes in an unpredictable way.

### Example 39. Transcriptional Changes in Recombinant Cac Strains Upon Interaction with Clj in Coculture

As reported above, we noted the sudden growth cessation that occurs at 24 hours into the cocultures of the recombinant CACas9 (-hbd)/p95ace02a (which constitutively expresses the acetone pathway from Cac: ctfA, ctfB, thl, adc) and Clj/p100ptaHalo. From here on in this example, CACas9 (-hbd)/p95ace02a will be referred to as “recombinant Cac”, Clj/p100ptaHalo will be referred to as “Clj Halo” and wild-type versions of either bacteria will be referred to as Cac WT or Clj WT. To gain a deeper understanding of the transcriptional changes in the recombinant Cac in response to the coculture environment, we performed transcriptional studies using RNA Seq on the coculture samples at around 12 and 24 hours into the coculture, along with sampling of the recombinant Cac and Clj Halo monocultures at the same time points. From this information, we sought to develop a profile of the most significant differentially expressed genes when recombinant Cac is in coculture with Clj Halo (in comparison to recombinant Cac in monoculture). In addition, we sought to compare these data to previously obtained data in our lab showcasing the significant differentially expressed genes when Cac WT is in coculture with Clj WT versus its behavior in monoculture.

Among upregulated genes in the recombinant Cac, there were ABC transporters related to phosphate uptake, including phoU, a phosphate uptake regulator with a fold change of 4.68. In the Cac WT results, genes of the phosphate uptake category were not significantly differentially expressed in the coculture compared to the monoculture. In addition, we compared the recombinant Cac's top significantly downregulated genes when in coculture compared to monoculture, as well as the same condition for the wild-type Cac. The recombinant Cac's leuA was downregulated 5.70-fold when in the coculture, versus only 1-fold (which is not a significant change) in WT Cac. There were also genes significantly downregulated in recombinant Cac (more than 4-fold) that were identified to be related to quorum sensing in Cac. However, genes of a similar nature did not rank among the top downregulated genes for the Cac WT study.

Of interest in the top downregulated genes in the recombinant Cac when in coculture was RNA polymerase factor sigma 70, with extracytoplasmic function. This gene (CA_C1766) had been previously studied in detail along with other clostridial sporulation factors of unknown function. Antisense RNA (asRNA) targeting this gene was performed, along with transmission electron microscopy of the asCA_C1766 cells. The results revealed pinched portions of the cell membrane around the endospore forming cells, uncharacteristic of the Cac WT. From these images, the authors suggested that CA_C1766 played a role in the development of spores in Cac due to the altered phenotypes. Thus, it is significant that in the recombinant Cac strain, this sigma factor would be significantly downregulated (4-fold). In previous studies with the Cac WT, this sigma factor, as well as others known to be involved in sporulation, did not experience significant differential expression at a large fold-change (>1).

To sum, our RNAseq results demonstrate very significant differences in gene regulation in the WT Cac vs CACas9Δhbd/p95ace02a strain, and as suggested above, these changes make it impossible to predict the behavior of the genetic modifications of one or both strains in coculture from the cocultures of WT Cac and Clj strains, and notably in their ability to interact directly, fuse and exchange electrons, metabolites and cellular material. The cellular machinery that is responsible for such events is affected by unrelated genetic modifications.

### Example 40. Carbohydrate-Consuming Solventogenic Clostridium Microorganisms

Clostridium acetobutylicum is a model microorganism for solventogenic Clostridium microorganisms, defined as microorganisms that consume a broad range of simple and complex carbohydrates to produce solvents, notably short and longer chain alcohols (e.g. butanol & ethanol), their corresponding carboxylic acids and additional 3 and 4-C metabolites, such as acetoin, acetone or isopropanol. Not all such microorganisms produce the same mix of solvent molecules. Clostridium acetobutylicum was selected as the model microorganism representing most such microorganisms in terms of core metabolic pathways and regulation including sporulation (Jones, Paredes et al. 2008, Papoutsakis 2008, Tracy, Jones et al. 2012, Charubin, Gregory et al. 2021). This group of microorganisms is defined by their similar metabolic pathways and capabilities, and a person skilled in the art using current scientific knowledge including the scientific findings summarized in the cited references above, will conclude that several of these microorganisms will meet the capabilities of our current claims. In addition to Clostridium acetobutylicum, this group would include, but is not limited to Clostridium beijerinckii, Clostridium saccharolyticum and Clostridium butyricum (Papoutsakis 2008, Tracy, Jones et al. 2012). Due to their similar genes and cell regulation, they are assumed to have the necessary cellular machinery to enable cell fusion and the unique capabilities demonstrated by this C. acetobutylicum/C. ljungdahlii coculture, and upon similar genetic modifications, will retain the ability for heterologous cell fusion with Clostridium ljungdahlii and other acetogens.

### Example 41. Acetogenic Microorganisms with Similar Genetic and Metabolic Characteristics

Clostridium ljungdahlii was selected from a group of like acetogens (acetogenic microorganisms) for their ability to consume CO₂ and H₂ among autotrophically using the Wood-Ljungdahl Pathway (Tracy, Jones et al. 2012). This group of microorganisms is defined by their similar metabolic pathways and capabilities, and we anticipate that other microorganisms will meet the capabilities of our current claims. In addition to Clostridium ljungdahlii, this group would include, but is not limited to Clostridium autoethanogenum, Clostridium carboxidivorans, Acetobacterium woodii, and Eubacterium limosum (Papoutsakis 2008, Tracy, Jones et al. 2012). Due to their similar genes and cell regulation, they are assumed to have the necessary cellular machinery to enable cell fusion and the unique capabilities demonstrated by this C. acetobutylicum/C. ljungdahlii coculture.

### Example 42. Direct Cell-to-Cell Contact in the Coculture System Profoundly Impacts the Transcriptome of the Two Microorganisms and Identifies Differential Expressed Genes that Appear Physiologically Relevant to the Phenotype

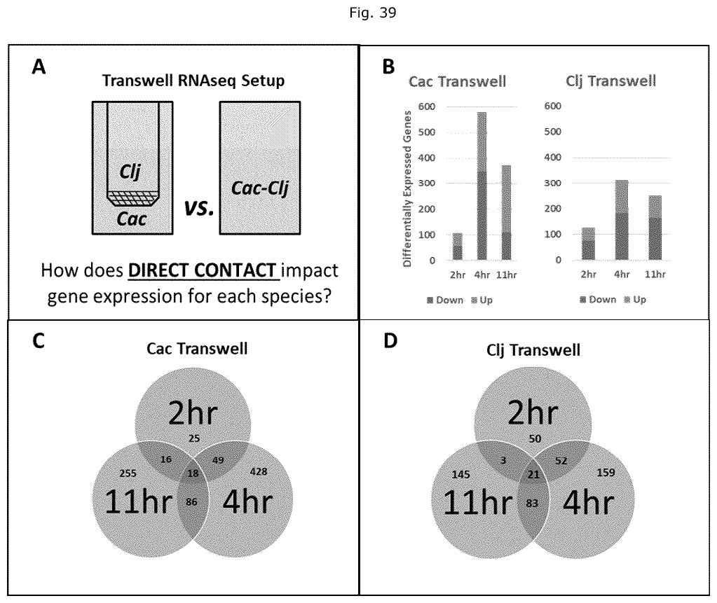

We found that growing a coculture of Cac and Clj in a transwell system prevents cell-to-cell contact, alters the metabolic profile and abolishes the fluorescent material exchange that occur when the two microorganisms are grown together (Charubin and Papoutsakis 2019). We have used the same transwell system (FIG. 39A) in combination with RNAseq to identify genes in both microorganisms are necessary or involved in heterologous cell fusion leading to direct cellular material, electron and metabolite exchange. To do so, we determined the genes that are differentially expressed when the cells of the two species can directly interact compared to when they share the same medium but are separated by a membrane (FIGS. 39B, C & D). If the two cell types (Cac and Clj) interact only through the culture medium components and secreted metabolites or other molecules, then there would be no difference in gene expression between the separated or transwell separated cultures. However, we found profound differences in the differential gene expression (FIGS. 39B, C & D) . . . . Thus, the differentially expressed genes include the genes that enable and are necessary in heterologous cell fusion leading to direct cellular material, electron and metabolite exchange. We examined the transcriptome of the coculture at 2, 4, and 11 hr timepoints. Both Cac and Clj had the largest number of differentially expressed genes at 4 hours (FIG. 39B). These results support our previous flow cytometry studies (Charubin, Modla et al. 2020) which suggest that cell fusion events begin between 2 and 4 hours and fusion frequency peaks between 4 and 11 hours. Many of these proteins are consistent with expectations as discussed in our publication (Charubin, Gregory et al. 2021), and this information is supporting the value of these RNAseq studies. However, as we genetically modify the two strains (Cac and Clj), related and seemingly unrelated genetic modification affect directly or indirectly hundreds of other genes as we have documented (Tomas, Alsaker et al. 2003, Tomas, Welker et al. 2003, Alsaker, Spitzer et al. 2004, Tomas, Beamish et al. 2004, Jones, Paredes et al. 2008, Jones, Tracy et al. 2011, Tracy, Jones et al. 2011) and thus one cannot predict if genetically modified strains will still enable heterologous cell fusion with suitable partners under the specific co-culture conditions disclosed in this invention, since as we have shown in Examples 37 and 38, the hbd-deleted M5 strain cannot form heterologous cell fusion with Clj, while the parent M5 strain can.

Significantly, not any pair of microorganism when co-cultured will form heterologous cell fusions. In fact, few pairs may, but one cannot predict a priori which pairs will or will not. For example, the pairs of Escherichia coli (WT or genetically modified) will not fuse with Bacillus subtilis in co-culture. Also, Clostridium ljungdahlii will not fuse with Clostridium carboxydivorans in coculture.

Heterologous cell fusion of genetically modified strains in co-culture as disclosed here require specific genetic modifications, and the disclosed culture conditions and culture media. Not every pair of microorganisms put in co-culture will form cell fusions. Understanding heterologous cell fusion is not very well developed, but specific cellular machinery is necessary in each of the two microorganisms (Charubin, Gregory et al. 2021). This machinery is present and active in some but not all pairs of co-culture strains disclosed here. The pairs of strains of the solventogenic/acetogenic microorganisms disclosed in Examples 40 and 41 have the same machinery as the pair of (modified Cac)/Clj cells disclosed here and form cell fusions as well to enable direct transfer of cellular material and electrons to enable the technology detailed here based on the genetically modified Cac paired with Clj.

### REFERENCES

Al-Hinai, M. A., A. G. Fast and E. T. Papoutsakis (2012). “Novel system for efficient isolation of Clostridium double-crossover allelic exchange mutants enabling markerless chromosomal gene deletions and DNA integration.” Applied and environmental microbiology 78 (22): 8112-8121.
Alsaker, K. V., T. R. Spitzer and E. T. Papoutsakis (2004). “Transcriptional analysis of spo0A overexpression in Clostridium acetobutylicum and its effect on the cell's response to butanol stress.” Journal of Bacteriology 186 (7): 1959-1971.
Charubin, K., R. K. Bennett, A. G. Fast and E. T. Papoutsakis (2018). “Engineering Clostridium organisms as microbial cell-factories: challenges & opportunities.” Metabolic Engineering 50:173-191.
Charubin, K., G. J. Gregory and E. T. Papoutsakis (2021). Novel mechanism of plasmid-DNA transfer mediated by heterologous cell fusion in syntrophic coculture of Clostridium organisms, Cold Spring Harbor Laboratory: 10.1101/2021.1112.1115.472834.
Charubin, K., S. Modla, J. Caplan and E. Papoutsakis (2020). “Interspecies Microbial Fusion and Large-Scale Exchange of Cytoplasmic Proteins and RNA in a Syntrophic Clostridium Coculture.” mBio 11, e02030-20 (5): 10.1128/mBio.02030-02020.
Charubin, K. and E. T. Papoutsakis (2019). “Direct cell-to-cell exchange of matter in a synthetic Clostridium syntrophy enables CO₂ fixation, superior metabolite yields, and an expanded metabolic space.” Metab Eng 52:9-19.
Harris, L. M., N. E. Welker and E. T. Papoutsakis (2002). “Northern, morphological, and fermentation analysis of <i>spo0A</i> inactivation and overexpression in <i>Clostridium acetobutylicum</i> ATCC 824.” Journal of Bacteriology 184 (13): 3586-3597.
Harris, L. M., N. E. Welker and E. T. Papoutsakis (2002). “Northern, morphological, and fermentation analysis of spoOA inactivation and overexpression in Clostridium acetobutylicum ATCC 824.” J Bacteriol 184 (13): 3586-3597.
Jones, S. W., C. J. Paredes, B. Tracy, N. Cheng, R. Sillers, R. S. Senger and E. T. Papoutsakis (2008). “The transcriptional program underlying the physiology of clostridial sporulation.” Genome Biology 9 (7): R114. DOI110.1186/gb-2008-1189-1187-r1114.
Jones, S. W., B. P. Tracy, S. M. Gaida and E. T. Papoutsakis (2011). “Inactivation of sigma (F) in Clostridium acetobutylicum ATCC 824 Blocks Sporulation Prior to Asymmetric Division and Abolishes sigma (E) and sigma (G) Protein Expression but Does Not Block Solvent Formation.” Journal of Bacteriology 193 (10): 2429-2440.
Kim, B. H., P. Bellows, R. Datta and J. G. Zeikus (1984). “CONTROL OF CARBON AND ELECTRON FLOW IN CLOSTRIDIUM-ACETOBUTYLICUM FERMENTATIONS-UTILIZATION OF CARBON-MONOXIDE TO INHIBIT HYDROGEN-PRODUCTION AND TO ENHANCE BUTANOL YIELDS.” Applied and Environmental Microbiology 48 (4): 764-770.
Papoutsakis, E. T. (2008). “Engineering solventogenic clostridia.” Current Opinion in Biotechnology 19 (5): 420-429.
Papoutsakis, E. T. (2008). “Engineering solventogenic clostridia.” Curr Opin Biotechnol 19 (5): 420-429.
Ragsdale, S. W. and E. Pierce (2008). “Acetogenesis and the Wood-Ljungdahl pathway of CO₂ fixation.” Biochimica et Biophysica Acta (BBA)-Proteins and Proteomics 1784 (12): 1873-1898.
Tomas, C. A., K. V. Alsaker, H. P. J. Bonarius, W. T. Hendriksen, H. Yang, J. A. Beamish, C. J. Paredes and E. T. Papoutsakis (2003). “DNA array-based transcriptional analysis of asporogenous, nonsolventogenic Clostridium acetobutylicum strains SKO1 and M5.” Journal of Bacteriology 185 (15): 4539-4547.
Tomas, C. A., J. Beamish and E. T. Papoutsakis (2004). “Transcriptional analysis of butanol stress and tolerance in Clostridium acetobutylicum.” Journal of Bacteriology 186 (7): 2006-2018.
Tomas, C. A., N. E. Welker and E. T. Papoutsakis (2003). “Overexpression of groESL in Clostridium acetobutylicum results in increased solvent production and tolerance, prolonged metabolism, and changes in the cell's transcriptional program.” Applied and Environmental Microbiology 69 (8): 4951-4965.
Tracy, B. P., S. W. Jones, A. G. Fast, D. C. Indurthi and E. T. Papoutsakis (2012). “Clostridia: the importance of their exceptional substrate and metabolite diversity for biofuel and biorefinery applications.” Current Opinion in Biotechnology 23 (3): 364-381.
Tracy, B. P., S. W. Jones and E. T. Papoutsakis (2011). “Inactivation of sigma (E) and sigma (G) in Clostridium acetobutylicum Illuminates Their Roles in Clostridial-Cell-Form Biogenesis, Granulose Synthesis, Solventogenesis, and Spore Morphogenesis.” Journal of Bacteriology 193 (6): 1414-1426.
Yoo, M., G. Bestel-Corre, C. Croux, A. Riviere, I. Meynial-Salles and P. Soucaille (2015). “A Quantitative System-Scale Characterization of the Metabolism of Clostridium acetobutylicum.” mBio 6 (6): e01808-01815.

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1. A method for producing metabolites by a co-culture comprising a first microorganism and a second microorganism in a medium, wherein the first microorganism is different from the second microorganism, the method comprising:
(a) growing the first microorganism and the second microorganism in the medium, wherein the medium comprises one or more exogenous carbohydrates, exogenous H₂, and exogenous CO₂;
(b) consuming the one or more exogenous carbohydrates by the first microorganism, and producing acetone, first acetate, CO₂ and H₂ by the first microorganism, wherein the first microorganism does not utilize for cell growth exogenous CO₂ or exogenous H₂, and does not produce butyrate, crotonate, butanol, butyraldehyde, beta-hydroxybutyrate or a long carbon chain length carboxylic acid;
(c) consuming the produced acetone, the produced CO₂, the produced H₂, the exogenous CO₂, and the exogenous H₂ by the second microorganism, and producing isopropanol, second acetate, and ethanol by the second microorganism, wherein the second microorganism does not produce CO₂ or H₂;
(d) consuming the second produced acetate by the first microorganism, and producing acetyl-CoA by the first microorganism; and
(e) forming cell membrane fusion by the first microorganism and the second microorganism, wherein electrons, the produced acetone, the produced CO₂ and the produced H₂ are transferred from the first microorganism into the second microorganism via the cell membrane fusion, and wherein the second produced acetate is transferred from the second microorganism into the first microorganism via the cell membrane fusion, whereby the co-culture produces the metabolites in the medium, wherein the metabolites comprise the produced acetone, the produced isopropanol, the produced ethanol, the produced first acetate, and the produced second acetate.

2. The method of claim 1, further comprising growing the co-culture anaerobically or micro-aerobically.

3. The method of claim 1, wherein the one or more exogenous carbohydrates comprise starch, glucose, xylose, fructose, hemicellulose, arabinose, or a combination thereof.

4. The method of claim 1, wherein the first microorganism is a recombinant Clostridium acetobutylicum (Cac), and the second microorganism is Clostridium ljungdahlii (Clj).

5. The method of claim 4, wherein the one or more exogenous carbohydrates consist of glucose, the method further comprising consuming the glucose by the Cac, and consuming the exogenous CO₂ by the Clj.

6. The method of claim 5, wherein at least 70% of the carbon atoms in the consumed glucose are transferred into the produced isopropanol, and less than 25% of the carbon atoms in the consumed glucose are transferred into the produced ethanol.

7. The method of claim 5, wherein the molar ratio of the consumed glucose to the consumed exogenous CO₂ is from 2:1 to 1:6.

8. The method of claim 5, wherein 10% to 80% of the carbon atoms in the metabolites for each mol of the consumed glucose are from the consumed exogenous CO₂.

9. The method of claim 4, wherein the Cac overexpresses one or more enzymes selected from the group consisting of Acetoacetyl-CoA:acetate/butyrate CoA transferases (CTFA&B), thiolase (THL), acetoacetate decarboxylase (ADC), secondary alcohol dehydrogenase (SADH), and a combination thereof.

10. The method of claim 4, wherein the Clj overexpresses one or more enzymes selected from the group consisting of Acetoacetyl-CoA:acetate/butyrate CoA transferases (CTFA&B), thiolase (THL), acetoacetate decarboxylase (ADC), secondary alcohol dehydrogenase (SADH), and a combination thereof.

11. The method of claim 4, wherein the Cac overexpresses one or more enzymes selected from the group consisting of THL of Escherichia coli (NCBI Reference Sequence: WP_000786547.1), CTFA&B of Escherichia coli (NCBI Reference Sequence: WP_000850540.1 and WP_000339065.1) or Clostridium acetobutylicum (NCBI Reference Sequence: WP_010890847.1 and WP_010890848.1), SADH of Clostridium beijerinckii (NCBI Reference Sequence: WP_077844196.1), and a combination thereof.

12. The method of claim 4, wherein the Clj overexpresses one or more enzymes selected from the group consisting of THL of Escherichia coli (NCBI Reference Sequence: WP_000786547.1), CTFA&B of Escherichia coli (NCBI Reference Sequence: WP_000850540.1 and WP_000339065.1) or Clostridium acetobutylicum (NCBI Reference Sequence: WP_010890847.1 and WP_010890848.1), SADH of Clostridium beijerinckii (NCBI Reference Sequence: WP_077844196.1), and a combination thereof.

13. The method of claim 4, wherein spCas9 from Streptococcus Pyogenes (NCBI Reference Sequence: WP_010922251.1) is integrated into a chromosome of the Cac at the location of the endogenous lactate dehydrogenase gene (ldhA, Locus tag: CA_C0267), the method further comprising disrupting the function of the ldhA in Cac.

14. The method of claim 13, wherein endogenous 3-hydroxybutyryl-CoA dehydrogenase gene (hbd, Locus tag: CA_C2708) is deleted from the chromosome or inactivated in the Cac.

15. The method of claim 13, wherein endogenous short-chain-enoyl-CoA hydratase gene (crt, Locus tag: CA_C2712) is deleted from the chromosome or inactivated in the Cac.

16. The method of claim 13, wherein endogenous acyl-CoA dehydrogenase gene (bcd, Locus tag: CA_C2711) is deleted from the chromosome or inactivated in the Cac.

17. The method of claim 13, wherein the medium further comprises exogenous butyrate at a concentration of 1-200 mM, the method further comprising enhancing the cell growth of the Cac.

18. The method of claim 13, wherein the medium further comprises exogenous crotonate at a concentration of 1-200 mM, the method further comprising enhancing the cell growth of the Cac.

19-25. (canceled)

26. A recombinant microorganism derived from a microorganism selected from the group consisting of Clostridium acetobutylicum, Clostridium ljungdahlii, Eubacterium limosum, Clostridium beijerinckii, Clostridium autoethanogenum, Acetobacterium woodii, Clostridium saccharolyticum, Clostridium butyricum and Clostridium carboxydivorans, the recombinant microorganism overexpressing:
(a) one or more enzymes selected from the group consisting of Acetoacetyl-CoA:acetate/butyrate CoA transferases (CTFA&B), thiolase (THL), acetoacetate decarboxylase (ADC), secondary alcohol dehydrogenase (SADH), and a combination thereof; and/or
(b) one or more enzymes selected from the group consisting of THL of Escherichia coli (NCBI Reference Sequence: WP_000786547.1), CTFA&B of Escherichia coli (NCBI Reference Sequence: WP_000850540.1 and WP_000339065.1) or Clostridium acetobutylicum (NCBI Reference Sequence: WP_010890847.1 and WP_010890848.1), SADH of Clostridium beijerinckii (NCBI Reference Sequence: WP_077844196.1), and a combination thereof.

27-30. (canceled)

31. A co-culture for producing metabolites, comprising the recombinant microorganism of claim 26 and an additional microorganism in a medium,
wherein the medium comprises one or more exogenous carbohydrates, exogenous H₂, and exogenous CO₂;
wherein the recombinant microorganism is different from the additional microorganism,
wherein the recombinant microorganism consumes the one or more exogenous carbohydrates, and produces acetone, first acetate, butanol, CO₂ and H₂;
wherein the additional microorganism consumes the produced acetone, the produced CO₂, the produced H₂, the exogenous CO₂, and the exogenous H₂, and produces isopropanol, second acetate, and ethanol;
wherein the recombinant microorganism consumes the second produced acetate and produces acetyl-CoA; and
wherein the recombinant microorganism and the additional microorganism form cell membrane fusion, wherein electrons, the produced acetone, the produced CO₂ and the produced H₂ are transferred from the recombinant microorganism into the additional microorganism via the cell membrane fusion, and wherein the second produced acetate is transferred from the additional microorganism into the recombinant microorganism via the cell membrane fusion, and
wherein the metabolites comprise the produced acetone, the produced isopropanol, the produced ethanol, the produced first acetate, the produced butanol, and the produced second acetate.

32-40. (canceled)
